(19) United States
(12) Patent Application Publication
(10) Pub. No.: US 2022/0396635 A1
(43) Pub. Date: Dec. 15, 2022

(54) TRIVALENT BINDING MOLECULES

(71) Applicant: UNIVERSITÄT STUTTGART, Stuttgart (DE)

(72) Inventors: Roland KONTERMANN, Nürtingen (DE); Oliver SEIFERT, Stuttgart (DE); Nadine ASCHMONEIT, Stuttgart (DE)

(21) Appl. No.: 17/762,626

(22) PCT Filed: Sep. 25, 2020
(86) PCT No.: PCT/EP2020/077012

§ 371 Date: Mar. 22, 2022

(30) Foreign Application Priority Data
Sep. 25, 2019 (EP) 19199641.2

(51) Int. Cl. C07K 16/32; C07K 16/28

(52) U.S. Cl. CPC C07K 16/32; C07K 16/2803; C07K 2317/31; C07K 2317/35; C07K 2317/622; C07K 2317/626; C07K 2317/70; C07K 2317/24

(57) ABSTRACT

The present invention relates to a trivalent binding molecule comprising a first polypeptide comprising two binding domains and a second polypeptide comprising a third binding domain. The present invention further relates to the trivalent binding molecule for use in medicine and in particular in the prophylaxis, treatment or diagnosis of a disorder or disease.

### TRIVALENT BINDING MOLECULES

The present invention relates to a trivalent binding molecule comprising a first polypeptide comprising two binding domains and a second polypeptide comprising a third binding domain. The present invention further relates to the trivalent binding molecule for use in medicine and in particular in the prophylaxis, treatment or diagnosis of a disorder or disease.

### BACKGROUND OF THE INVENTION

Monoclonal antibodies have become an established treatment modality for a variety of diseases. Antibody engineering is routinely applied to adapt the composition and activity for therapeutic applications in humans, including a reduction of immunogenicity generating chimeric, humanized or fully human antibodies and the modification of Fc-mediated effector functions, e.g. increasing or abrogating ADCC (Presta, L G. 2008, Molecular engineering and design of therapeutic antibodies. Curr Opin. Immunol. 20, 460-470). Monoclonal antibodies possess a defined specificity for a single epitope of an antigen, and can thus address a singular target only. However, complex diseases such as cancer or inflammatory disorders are usually multifactorial in nature. This is reflected by a redundancy of disease-mediating ligands and receptors as well as crosstalk between signal cascades. For example, several proinflammatory cytokines such as TNF, IL-1 and IL-6 have been identified as key players in inflammatory diseases. In cancer, tumor cells often upregulate different growth-promoting receptors, which can either act independently or crosstalk intracellularly through signaling networks. Of note, an acquisition of resistance to therapy is often associated with upregulation of alternative receptors as well as pathway switching between two receptors. Consequently, therapy with monoclonal antibodies targeting a singular antigen only has its limitations.

Bi— and multispecific antibodies find increasing interest for diagnostic and therapeutic applications (Kontermann, 2012, Dual targeting strategies with bispecific antibodies, mAbs 4, 182-197). Bispecific and multispecific antibodies recognize two or more different epitopes either on the same or on different antigens (Garber K. Bispecific antibodies rise again. Nat. Rev. Drug Discov. 2014; 13:799-801; Brinkmann & Kontermann, 2017, The making of bispecific antibodies, mAbs 9, 182-212).

A large number of bispecific antibodies have been developed for the retargeting of immune effector cells to target cells with the aim to destroy the target cells by cytotoxic mechanisms of the effector cell. Several of these bispecific antibodies have already entered clinical development (Kontermann & Brinkmann, 2015, mAbs 9:182-212; Dahlén et al., 2018, Ther. Adv. Vaccines Immunother. 6:3-17; Yu & Wang, 2019, J. Cancer Res. Clin. Oncol. 145:941-956). T-cells are the main effector cells employed for this approach. T-cells are highly effective in killing target cells through recognition of MHC-presented peptides by T-cell receptors (TCR) with specificity for this peptide in the context of an MHC presentation. However, T-cells cannot be recruited to targeted cells by normal immunoglobulins (antibodies) due to the lack of Fc receptors. Bispecific antibodies are designed to bind simultaneously to a trigger molecule on the effector cell and a surface antigen on the target cell. Thus, this type of bispecific antibodies act as mediators to bring effector and target cells into close proximity and to activate the effector cell by triggering cytotoxic immune responses (Clynes & Desjarlais, 2018, Annu. Rev. Med. 70:437-450). For T-cells, CD3 is the most commonly used trigger molecule (Yu & Wang, 2019, J. Cancer Res. Clin. Oncol. 145:941-956), however, other molecules on T-cells such as CD2, CD5, CD44, CD69, Me114 and Ly-6.2C have been employed (Tutt et al., 1991, Eur. J. Immunol. 21:1351-1358; Tita-Nwa et al., 2007, Cancer Immunol. Immunother. 56:1911.1920; Segal et al., in Fanger, M. W. “Bispecific Antibodies” 1995, MBIU RG Landes, pp 27-42). Furthermore, this approach is also applicable for the retargeting and activation of other effector cells, such as natural killer cells and granulocytes, e.g. through binding to Fc receptors (e.g. CD16, CD64, CD89) on the effector cells (van Spriel et al., 2000, Immunol. Today 21:391-397).

Monovalent binding to the trigger molecule, e.g. CD3 on T-cells, has been identified as a prerequisite to avoid a systemic activation of T-cells and to restrict receptor cross-linking and T-cell activation to target cell-bound molecules (Segal et al., 1999, Curr. Opin. Immunol. 11:558-562; Husain & Ellerman, 2018, BioDrugs 32:441-464). Furthermore, it has been postulated that molecules should be devoid of Fc effector functions, e.g. by omitting an Fc region or using a mutated Fc region, to avoid an overshooting activation of accessory immune cells and a detrimental cytokine storm (Shimabukuro-Vornhagen et al., 2018, J. Immunother. Cancer 6:56). Respective antibody constructs include bispecific IgG molecules and F(ab′)2 fragments thereof, which have been, for instance, generated by either fusing two antibody producing hybridoma cells into a hybrid hybridoma (quadroma) or by chemical conjugation of two Fab′ fragments (Staerz & Bevan, 1986, Proc. Natl. Acad. Sci. USA 83:1453-1457; Graziano & Guptill, 2004, Methods Mol. Biol. 283:71-85). Catumaxomab is such a bispecific IgG antibody, derived from fusing mouse and rat hybridomas directed against EpCAM and CD3, respectively (Seimetz, 2011, J. Cancer 2:309-316). Catumaxomab was approved in 2009 for the treatment of malignant ascites in patients with EpCAM-positive tumors. Catumaxomab was withdraw from the market in 2017. Several drawbacks of this type of antibody have been identified, such as strong immunogenicity due to the non-human nature of the antibody (Ruf et al., 2010, Br. J. Clin. Pharmacol. 69:617-625), and a strong accessory immune cell activation due to the presence of an unmodified Fc region (Borlak et al., 2015, Oncotarget 7:28059-28074). This has led to development of a variety of genetically engineered bivalent, bispecific molecules. The vast majority of bispecific antibodies for effector cell retargeting utilize a 1+1 format, i.e. having one binding site for a target antigen and another for a trigger molecule on an immune effector cell, e.g. CD3 on T-cells as part of the T-cell receptor (TCR) (Brinkmann & Kontermann, 2017, mAbs 9:182-212; Labrijn et al., 2019, Nat. Rev. Drug. Discov. 18: 585-608). An example for a genetically engineered bispecific antibody approved for therapy is blinatumomab (Blincyto), which was approved in 2014 for the treatment of acute lymphoblastic leukemia (ALL) (Yu & Wang, 2019, J. Cancer Res. Clin. Oncol. 145:941-956). Blinatumomab is a small bispecific antibody molecule composed of two scFv fragments linked together by a short peptide linker (tandem scFv). The small distance between the two antigen-binding sites leads to close linkage of target and effector cell and efficient T-cell-mediated target cell killing, as shown for molecules targeting antigens on hematologic and solid tumors (Ellerman, 2019, Methods 154:102-117).

More recently, bispecific antibodies have been developed binding bivalently to tumor associated antigens (TAAs) on target cells and monovalently to CD3 on T-cells, i.e. exhibiting a 2+1 stoichiometry (Brinkmann & Kontermann, 2017, mAbs 9:182-212). The motivation for this approach is to retain the bivalent binding mode of IgG molecules, that is, utilizing avidity effects for binding to cell surface antigens, which can result in an avidity-mediated specificity gain (Vauquelin & Charlton, 2012, Br. J. Pharmacol. 168:1771-17785; Slaga et al., 2018, Sci. Transl. Med. 10:463). Furthermore, it allows to implement binding to two different epitopes/antigens on a target cells for improving target cell specificity.

Using the CrossMab technology, trivalent, bispecific molecules (TCB-T cell bispecific antibody) were generated fusing a Fab fragment to a bispecific IgG molecule (Klein et al., 2016, mAbs 8:1010-1020). These molecules were directed either against CEA or BCMA as tumor cell antigen, respectively (Bacac et al., 2016, Onco Immunol. 5:e1203498; Seckinger et al., 2017, Cancer Cell 31:396-410). However, these molecules are rather large in size (a Fab-IgG molecule has a size of approx. 200 kDa) and require four different polypeptide chains to be produced and correctly assembled into a trivalent, bispecific molecule.

Another example is a trispecific TRIDENT molecule composed of a DART (a bispecific diabody derivative) and a Fab fragment dimerized through an Fc region (Liu et al., 2019, tumor-antigen 5T4-dependent activation of the CD137 costimulatory pathway by bispecific 5T4×CD137 × CD137 TRIDENT molecules. AACR 2019, Abstract 3476). Although smaller in size than a Fab-IgG molecule, it also requires expression of four different polypeptide chains to be produced and correctly assembled into a trivalent, bispecific molecule.

Other trivalent bispecific molecules developed include i) triplebodies (Schubert et al., 2011, mAbs 3:21-30; Roskopf et al., 2016, Onoctarget 7:22579-22589), which are composed of three scFv molecules connected by two linkers (which bear the risk of mispairing between the different VH and VL domains of the three scFv due to highly flexible linkage), ii) Fab-scFv₂ molecules (Tribodies) (Schoonjans et al., 2000, J. Immunol. 165:7050-7057; Schoonjans et al., 2001, Biomol. Eng. 17:193-202) generated by fusing scFv fragments to the C-terminus of the light chain and heavy chain of a Fab fragment, iii) molecules composed of two Fab fragments connected to a scFv molecule by the dock-and-lock (DNL) method (Rossi et al., 2014, mAbs 6:381-391), and iv) Tri-Fabs composed of two identical Fab arms fused to a VH-CH3/VL-CH3 module with knobs-into-holes mutations in the CH3 domains to force heterodimerization (Dickopf et al. 2019, Biol. Chem. 400:343-350).

In summary, the currently available trivalent, bispecific antibodies suffer from one or more of the following disadvantages:
(1) they require three or more polypeptide chains to be produced and to be assembled into a bispecific molecule,
(2) they are large in size (>150 kDa),
(3) they lack an Fc region which facilitates purification by affinity chromatography,
(4) they have a low yield of the desired trivalent binding molecules due to mispairing; and/or
(5) they lack a rigid structure and a small distance between the binding sites for target and trigger molecule.

In view of the drawbacks of the prior art, the present inventors established a single-chain dual valence antigen binding polypeptide (scDVAP) as building block to generate trivalent, bispecific molecules solving the above described obstacles. The present invention thus provides a modular system composed of a first polypeptide comprising the scDVAP with two binding domains, and a second polypeptide comprising a third binding domain to generate trivalent bispecific molecules with (1) as little as two interconnected polypeptide chains, (2) a smaller size of usually below 150 kDa, (3) an optional Fc region, and (4) a rigid structure and a sufficiently large distance between the binding sites for a target and a trigger molecule.

### SUMMARY OF THE INVENTION

In a first aspect, the present invention provides a trivalent binding molecule comprising a first and a second polypeptide. The first polypeptide comprises a single-chain dual valence antigen binding polypeptide (scDVAP), wherein the scDVAP comprises a first binding domain comprising a first variable chain (VC1) and a second variable chain (VC2), and a second binding domain comprising a third variable chain (VC3) and a fourth variable chain (VC4), wherein VC1 and VC2 together form a first antigen binding site, and VC3 and VC4 together form a second antigen binding site, wherein either (i) VC1 and VC4 are connected by a first peptide linker (L1), VC4 and VC3 are connected by a second peptide linker (L2), and VC3 and VC2 are connected by a third peptide linker (L3), or (ii) VC4 and VC1 are connected by a first peptide linker (L1), VC1 and VC2 are connected by a second peptide linker (L2), and VC2 and VC3 are connected by a third peptide linker (L3). The second polypeptide comprises a third binding domain comprising a fifth variable chain (VC5) and a sixth variable chain (VC6), wherein VC5 and VC6 together form a third antigen binding site. According to the present invention, two of the binding sites of the trivalent binding molecule specifically bind to the same or different antigens which is not a trigger molecule on an immune effector cell. According to the present invention, only one of the binding sites of the trivalent binding molecule is directed against a trigger molecule on an immune effector cell. Further, according to the present invention the first and second polypeptide are interconnected.

According to one embodiment of the present invention, the first binding site of the scDVAP and the third binding site of the second polypeptide specifically bind the same or a different antigen, and the second binding site of the scDVAP specifically binds a trigger molecule on an immune effector cell. According to a different embodiment, the first binding site of the scDVAP and the second binding site of the scDVAP specifically bind the same or a different antigen, and the third binding site of the second binding module specifically binds a trigger molecule on an immune effector cell.

According to a preferred embodiment, the variable chains (VCs) are either based on a T cell receptor or on an antibody. Thus, each can be selected from the group consisting of an α-chain variable domain, β-chain variable domain, γ-chain variable domain, δ-chain variable domain, variable light chain domain (V_{L}) and variable heavy chain domain (V_{H}).

According to a further preferred embodiment, scDVAP is a single-chain diabody.

According to yet another embodiment, VC5 and VC6 are connected by a fourth peptide linker (L4).

According to another embodiment, the two binding sites bind the same antigen.

According to a further preferred embodiment, the second polypeptide is selected from the group consisting of a single variable heavy or light chain domain, an scFv, and a Fab fragment. According to yet another embodiment, the first and second polypeptides are interconnected by a fifth peptide linker (L5), a peptide bond, a disulfide bond or by one or more dimerization domains. According to a preferred embodiment, the one or more dimerization domain is selected from the group consisting of an Fc region, a heterodimerizing Fc region, C_{H}1, C_{L}, the second heavy chain constant domain (C_{H}2) of IgE and IgM (EHD2, MHD2), modified EHD2, the last heavy chain constant domain (C_{H}3 or C_{H}4) of IgG, IgD, IgA, IgM, or IgE and heterodimerizing derivatives thereof, and the constant domains C-α and C-β of a T-cell receptor. According to a preferred embodiment, the first binding module is connected, preferably via a peptide bond or a linker (L6), to a first heterodimerizing domain and the second binding module is connected, preferably via a peptide bond or a linker (L7), to a second heterodimerizing domain. According to a preferred embodiment, the heterodimerizing domains of the first and second polypeptide bind to each other through hydrophobic and/or electrostatic interactions.

According to yet another embodiment, the immune effector cell is selected from the group consisting of T-cells, natural killer cells, natural killer T cells, macrophages, and granulocytes.

According to yet another embodiment, the trigger molecule of the immune effector cell is selected from the group consisting of CD2, CD3, CD16, CD44, CD64, CD69, CD89, Mel14, or Ly-6.2C.

According to another embodiment, the antigen is a tumor-associated antigen, preferably wherein the tumor-associated antigen is selected from the group consisting of EGFR, EGFRvIII, HER2, HER3, HER4, cMET, RON, FGFR1, FGFR2, FGFR3, FGFR4, IGF-1R, AXL, Tyro-3 MerTK, ALK, ROS-1, ROR-1, ROR-2, RET, MCSP, FAP, Endoglin, EpCAM, claudin-6, claudin 18.2, CD19, CD20, CD22, CD30, CD33, CD52, CD38, CD123, BCMA, CEA, PSMA, DLL3, FLT3, gpA33, SLAM-7, CCR9.

According to a preferred embodiment the trivalent further comprising one or more of:
(a) a peptide leader sequence;
(b) one or more molecules that aid in purification, preferably a hexahistidyl-tag or FLAG-tag;
(c) one or more co-stimulatory molecules.

In a further aspect, the present invention relates to a nucleic acid or set of nucleic acids encoding the trivalent binding molecule.

The present invention also provides a vector comprising the nucleic acid or set of nucleic acids of the invention.

In a further aspect, the present invention relates to a pharmaceutical composition comprising the trivalent binding molecule of the invention, the nucleic acid or set of nucleic acids of the invention or the vector of the invention, and a pharmaceutically acceptable carrier.

In a further aspect, the present invention provides the trivalent binding molecule of the invention, the nucleic acid or set of nucleic acids of the invention, the vector of the invention or the pharmaceutical composition of the invention for use in medicine and/or for use in treating cancer, a viral infection or an autoimmune disease.

Further provided is a method of treating cancer, a viral infection or an autoimmune disease in a patient in need thereof, comprising administering to the patient the trivalent binding molecule of the invention, the nucleic acid or set of nucleic acids of the invention, the vector of the invention or the pharmaceutical composition of the invention.

In a further aspect, the present invention relates to method of inhibiting metastatic spread of a cell, comprising contacting the cell with the trivalent binding molecule of the invention, the nucleic acid or set of nucleic acids of the invention, the vector of the invention or the pharmaceutical composition of the invention.

According to a preferred embodiment, the cancer is selected from the group consisting of carcinoma, sarcoma, lymphoma, leukemia, germ cell tumor and blastoma.

### LIST OF FIGURES

In the following, the content of the figures comprised in this specification is described. In this context please also refer to the detailed description of the invention above and/or below.

FIG. 1: Schematic illustration of the building blocks used for the generation of binding molecules. (A) Binding module A (i and ii) are the single-chain dual valance antigen binding polypeptides (scDVAP) comprising the domains VC1, VC2, VC3, and VC4, and containing the antigen binding site 1 (VC1 and VC2) and 2 (VC3 and VC4). Examples of binding module B comprises VC5 and VC6 and form the antigen binding site 3, e.g. in the form of a Fv, scFv, Fab fragment, domain antibodies (dAb), or other derivatives thereof (B) A trivalent, bispecific binding molecule comprising binding module 1 and binding module 2 (VC5 and VC6 connected by linker L4) connected by a fifth linker L5. (C) Trivalent and bispecific derivatives obtained by fusing binding module 1 through a linker 6 (L6) to a first dimerization domain (DD1) and binding module 2 (exemplarily shown for a scFv and a Fab fragment) through a linker 7 (L7) to the same first dimerization domain (DD1) or a second dimerization domain (DD2), with the second binding module 2 either positioned at the same site (end) of the dimerization domains or the opposing site (end) of the dimerization domains.

FIG. 2: Biochemical characterization of scDb and scDb-scFv targeting HER3 and CD3. (A) Composition and schematic illustration of scDb and scDb-scFv. L, Igκ chain leader sequence. L1, G₄S; L2, (G₄S)₃; L3, G₄S; L4, (G₄S)₃; L5, AAAGGS(G₄S)GGGT. (B) SDS PAGE analysis (12% PAA, 2 μg/lane, Coomassie blue staining) of (1) scDb and (2) scDb-scFv under reducing (R) and non-reducing (NR) condition. M, protein marker. (C) Size-exclusion chromatography by HPLC using a Tosoh TSKgel SuperSW mAb HR column.

FIG. 3: Binding properties of scDb and scDb-scFv. Binding to HER3-expressing MCF-7 (A), LIM1215 (B), BT-474 (C), FaDu (D), and CD3-expressing Jurkat cells (E) was analyzed by flow cytometry. Bound protein was detected using a PE-labeled anti-His mAb. Mean±SD, n=3.

FIG. 4: Activity of scDb and scDb-scFv on cytokine release, early T-cell activation, and T-cell proliferation. (A) IFN-γ and IL-2 release mediated by scDb and scDb-scFv targeting HER3 and CD3. PBMCs were co-cultured with MCF-7 cells in presence of bsAb. After 24 h (IL-2) or 48 h (IFN-γ) supernatants were harvested and cytokine release was determined using sandwich ELSA. (B) CD69 expression on CD4⁺ and CD8⁺ T-cells was analyzed after PBMCs were co-cultured with MCF-7 cells in presence of bsAb for 24 h in flow cytometry. Mean±SD, n=3. (C) Proliferation of CD4⁺ and CD8⁺ T-cells. PBMCs were co-cultured with MCF-7 cells in presence of scDb or scDb-scFv for 6 days and proliferation of T-cells was measured by CFSE dilution by flow cytometry. Mean±SD, n=3. (D) Proliferation of naïve (T_{N}, CD45RA⁺, CCR7⁺), central memory (T_{CM}, CD45RA⁻, CCR7⁺), effector (T_{E}, CD45RA⁺, CCR7⁻) and effector memory (T_{EM}, CD45RA⁻, CCR7⁻) subpopulations of CD4⁺ T-cells and CD8⁺ T-cells was determined in flow cytometry. Mean±SD, n=3.

FIG. 5: Effect of scDb and scDb-scFv targeting HER3 and CD3 on cytotoxic potential of PBMCs. Target cells ((A, B) MCF-7, (C, D) LIM1215, (E, F) BT-474, or (G, H) FaDu cells) were incubated with a serial dilution of scDb or scDb-scFv and PBMCs in an effector:target cell ratio (E:T) of 10:1 or 5:1. After 3 days of incubation, cell viability was determined using crystal violet staining. Mean±SD, n=3.

FIG. 6: Schematic illustration of trivalent, bispecific anti-HER3×anti-CD3 antibodies. Composition and schematic illustration of trivalent, bispecific scDb/Fab-Fc (A) and scDb/scFv-Fc (B) fusion proteins. In dependence on the localization of the antigen binding sites, three different compositions of the trivalent antibodies are possible: (1-2)+1, (2-1)+1, and (1-1)+2. L, Igκ chain leader sequence. L1, G₄S; L2, (G₄S)₃; L3, G₄S; L4, (G₄S)₃; scDb, scFv or Fd fragments cloned to the Fc part using NotI as restriction enzyme resulting in three alanine residues between the antibody fragments and the Fc part.

FIG. 7: Biochemical characterization of trivalent, bispecific anti-HER3×anti-CD3 antibodies. (A) SDS PAGE analysis (12% PAA, 2 μg/lane, Coomassie blue staining) of scDb/scFv-Fc (1-2)+1 (1), scDb/Fab-Fc (1-2)+1 (2), scDb/scFv-Fc (1-1)+2 (3), scDb/Fab-Fc (1-1)+2 (4), scDb/scFv-Fc (2-1)+1(5), and scDb/Fab-Fc (2-1)+1 (6) under reducing (R) and non-reducing (NR) condition. (B) Size exclusion chromatography by HPLC using a Tosoh TSKgel SuperSW mAb HR column.

FIG. 8: Binding properties of trivalent, bispecific anti-HER3×anti-CD3 antibodies. Binding to HER3-expressing MCF-7(A), LIM1215 (B), and CD3-expressing Jurkat (C) cells was analyzed in flow cytometry. PE-labeled anti-human Fc antibody was used to detect bound protein. Mean±SD, n=3.

FIG. 9: Activity of trivalent, bispecific anti-HER3×anti-CD3 antibodies on T-cell proliferation. Proliferation of (A) CD8⁺ and (B) CD4⁺ T-cells mediated by trivalent, bispecific antibodies. PBMCs were co-cultured with MCF-7 cells in presence of fusion proteins. T-cell proliferation was analyzed after 6 days by CFSE dilution in flow cytometry. Mean±SD, n=3.

FIG. 10: Effect of trivalent, bispecific antibodies on cytotoxic potential of PBMCs. LIM1215 cells were incubated with a serial dilution of trivalent, bispecific antibodies in the scDb/sc-Fv-Fc or scDb/Fab-Fc format in presence of PBMCs in an effector:target cell ratio (E:T) of 10:1 (A), 5:1 (B), 2:1 (C). After 3 days of incubation at 37° C., cell viability was determined using crystal violet staining. Mean±SD, n=3.

FIG. 11: Biochemical characterization of scDb and scDb-scFv targeting EGFR and CD3. (A) SDS-PAGE analysis (12% PAA, 3 μg/lane, Coomassie blue staining) of (1) scDb and (2) scDb-scFv under reducing (R) and non-reducing (NR) condition. M, protein marker. (B) Size-exclusion chromatography by HPLC using a Tosoh TSKgel SuperSW mAb HR column.

FIG. 12: Binding properties of scDb and scDb-scFv targeting EGFR and CD3. Binding to EGFR-expressing FaDu (A), LIM1215 (B), SKBR-3 (C), T-47-D (D), and CD3-expressing Jurkat cells (E) was analyzed by flow cytometry. Bound protein was detected using a PE-labeled anti-His mAb. Mean±SD, n=3.

FIG. 13: Effect of scDb and scDb-scFv targeting EGFR and CD3 on cytotoxic potential of PBMCs. Target cells (FaDu (A), LIM1215 (B), SKBR-3 (C), or T-47-D (D)) were incubated with a serial dilution of scDb or scDb-scFv and PBMCs in an effector:target cell ratio (E:T) of 10:1. After 3 days of incubation, cell viability was determined using crystal violet staining. Mean±SD, n=3.

FIG. 14: Activity of scDb and scDb-scFv targeting EGFR and CD3 on T-cell proliferation of CD3⁺(A), CD4⁺(B) and CD8⁺ T-cells (C). PBMCs were co-cultured with FaDu cells in presence of scDb or scDb-scFv for 6 days and proliferation of T-cells was measured by CFSE dilution in flow cytometry. Mean±SD, n=3.

FIG. 15: Biochemical characterization of trivalent, bispecific anti-CEAxanti-CD3 antibodies. (A) SDS-PAGE analysis (12% PAA, 2 μg/lane, Coomassie blue staining) of scDb/Fab-Fc (1-2)+1 (1), scDb/Fab-Fc (2-1)+1 (2), scDb/Fab-Fc (1-1)+2 (3), scDb/scFv-Fc (1-2)+1 (4), scDb/scFv-Fc (2-1)+1 (5), and scDb/scFv-Fc (1-1)+2 (6) under reducing (R) and non-reducing (NR) condition. (B) Size-exclusion chromatography by HPLC using a Tosoh TSKgel SuperSW mAb HR column.

FIG. 16: Binding properties of trivalent, bispecific anti-CEAxanti-CD3 antibodies. Binding to CEA-expressing LIM1215 (A) and CD3-expressing Jurkat (B) cells was analyzed in flow cytometry. PE-labeled anti-human Fc antibody was used to detect bound protein. Mean±SD, n=3.

FIG. 17: Effect of trivalent, bispecific anti-CEAxanti-CD3 antibodies on cytotoxic potential of PBMCs. LIM1215 cells were incubated with a serial dilution of trivalent, bispecific antibodies in the scDb/sc-Fv-Fc or scDb/Fab-Fc format in presence of PBMCs in an effector:target cell ratio (E:T) of 10:1. After 3 days of incubation at 37° C., cell viability was determined using crystal violet staining. Mean±SEM, n=3.

FIG. 18: Activity of trivalent, bispecific anti-CEAxanti-CD3 antibodies on cytokine release. IL-2 release mediated by trivalent, bispecific antibodies. PBMCs were co-cultured with LIM1215 cells in presence of trivalent, bispecific antibodies. After 24 h supernatants were harvested and cytokine release was determined using sandwich ELSA.

FIG. 19: Biochemical characterization of trivalent, bispecific scDb/scFv-Fc and scDb/Fab-Fc molecules targeting EGFR and CD3. (A) SDS-PAGE analysis (12% PAA, 3 μg/lane, Coomassie blue staining) of (1) scDb/scFv-Fc (2-1)+1, (2) scDb/Fab-Fc (2-1)+1, (3) scDb/Fab-Fc (1-1)+2, (4) scDb/scFv-Fc (1-1)+2, (5) scDb/scFv-Fc (1-2)+1, (6) scDb/Fab-Fc (1-2)+1 under reducing (R) condition. M, protein marker. (B) Size-exclusion chromatography by HPLC using a Tosoh TSKgel SuperSW mAb HR column.

FIG. 20: Binding properties of trivalent, bispecific scDb/scFv-Fc and scDb/Fab-Fc molecules targeting EGFR and CD3. Binding to EGFR-expressing FaDu (A), LIM1215 (B), SKBR-3 (C), T-47-D (D), MCF-7 (E) and CD3-expressing Jurkat cells (F) was analyzed by flow cytometry. Bound protein was detected using a PE-labeled anti-huFc mAb. Mean±SD, n=3.

FIG. 21: Effect of trivalent, bispecific antibodies targeting EGFR and CD3 on cytotoxic potential of PBMCs. Target cells (FaDu (A) and SKBR-3 (B)) were incubated with a serial dilution of trivalent, bispecific antibodies and PBMCs in an effector:target cell ratio (E:T) of 5:1. After 3 days of incubation, cell viability was determined using crystal violet staining. Mean±SD, n=3.

FIG. 22: Biochemical characterization of trivalent, triispecific scDb/scFv-Fc and scDb/Fab-Fc molecules targeting EGFR, HER3 and CD3. (A) SDS-PAGE analysis (12% PAA, 3 μg/lane, Coomassie blue staining) of (1) scDb/scFv-Fc (2-1)+3, (2) scDb/Fab-Fc (2-1)+3, (3) scDb/Fab-Fc (1-3)+2, (4) scDb/scFv-Fc (1-3)+2, (5) scDb/scFv-Fc (1-2)+3, (6) scDb/Fab-Fc (1-2)+3 under reducing (R) and non-reducing (NR) condition. M, protein marker. (B) Size-exclusion chromatography by HPLC using a Tosoh TSKgel SuperSW mAb HR column.

FIG. 23: Binding properties of trivalent, trispecific antibodies targeting EGFR, HER3 and CD3. Binding to EGFR- and HER3-expressing FaDu (A), LIM1215 (B), SKBR-3 (C), T-47-D (D), MCF-7 (E) and CD3-expressing Jurkat cells (F) was analyzed by flow cytometry. Bound protein was detected using a PE-labeled anti-huFc mAb. Mean±SD, n=3.

FIG. 24: Effect of trivalent, trispecific antibodies targeting EGFR, HER3 and CD3 on cytotoxic potential of PBMCs. Target cells T-47-D were incubated with a serial dilution of the trivalent, trispecific antibodies and PBMCs in an effector:target cell ratio (E:T) of 5:1. After 3 days of incubation, cell viability was determined using crystal violet staining. Mean±SD, n=2.

FIG. 25: Binding properties of trivalent, bispecific antibodies targeting FAP and CD3. Binding to FAP-expressing HT1080-FAP cells was analyzed in flow cytometry. PE-labeled anti-human Fc antibody was used to detect bound protein. Mean±SD, n=3.

List of Sequences
SEQ ID NO: 1
GGGGS
SEQ ID NO: 2
([G₄S]₃)
GGGGSGGGGSGGGGS
SEQ ID NO: 3
AAAGGSGGGGSGGGT
SEQ ID NO: 4
(A₃)
AAA
(scDb3-43xhuU3-His)
SEQ ID NO: 5
QVQLQQSGPGLVKPSQTLSLTCAISGDSVSSNRAA
WNWIRQSPSRGLEWLGRTYYRSKWYNDYAQSLKSR
ITINPDTPKNQFSLQLNSVTPEDTAVYYCARDGQL
GLDALDIWGQGTMVTVSSGGGGSDIQMTQSPSSLS
ASVGDRVTITCRASQDIRNYLNWYQQKPGKAPKLL
IYYTSRLHSGVPSRFSGSGSGTDFTFTISSLQPED
IATYYCQQGNTLPWTFGQGTKLEIKRGGGGSGGGG
SGGGGSQVQLVQSGAEVKKPGSSVKVSCKASGGTF
SGYTMNWVRQAPGQGLEWMGLINPYKGVSTYNGKF
KDRVTITADKSTSTAYMELSSLRSEDTAVYYCARS
GYYGDSDWYFDVWGQGTLVTVSSGGGGSQAGLTQP
PAVSVAPGQTASITCGRDNIGSRSVHWYQQKPGQA
PVLVVYDDSDRPAGIPERFSGSNYENTATLTISRV
EAGDEADYYCQVWGITSDHVVFGGGTKLTVLAAAH
HHHHH
(scDb3-43xhuU3-scFv3-43-His)
SEQ ID NO: 6
QVQLQQSGPGLVKPSQTLSLTCAISGDSVSSNRAA
WNWIRQSPSRGLEWLGRTYYRSKWYNDYAQSLKSR
ITINPDTPKNQFSLQLNSVTPEDTAVYYCARDGQL
GLDALDIWGQGTMVTVSSGGGGSDIQMTQSPSSLS
ASVGDRVTITCRASQDIRNYLNWYQQKPGKAPKLL
IYYTSRLHSGVPSRFSGSGSGTDFTFTISSLQPED
IATYYCQQGNTLPWTFGQGTKLEIKRGGGGSGGGG
SGGGGSQVQLVQSGAEVKKPGSSVKVSCKASGGTF
SGYTMNWVRQAPGQGLEWMGLINPYKGVSTYNGKF
KDRVTITADKSTSTAYMELSSLRSEDTAVYYCARS
GYYGDSDWYFDVWGQGTLVTVSSGGGGSQAGLTQP
PAVSVAPGQTASITCGRDNIGSRSVHWYQQKPGQA
PVLVVYDDSDRPAGIPERFSGSNYENTATLTISRV
EAGDEADYYCQVWGITSDHVVFGGGTKLTVLAAAG
GSGGGGSGGGTQVQLQQSGPGLVKPSQTLSLTCAI
SGDSVSSNRAAWNWIRQSPSRGLEWLGRTYYRSKW
YNDYAQSLKSRITINPDTPKNQFSLQLNSVTPEDT
AVYYCARDGQLGLDALDIWGQGTMVTVSSGGGGSG
GGGSGGGGSQAGLTQPPAVSVAPGQTASITCGRDN
IGSRSVHWYQQKPGQAPVLVVYDDSDRPAGIPERF
SGSNYENTATLTISRVEAGDEADYYCQVWGITSDH
VVFGGGTKLTVLGSLHHHHHH
(scDb3-43xhuU3-Fc
(hole)Δab)
SEQ ID NO: 7
QVQLQQSGPGLVKPSQTLSLTCAISGDSVSSNRAA
WNWIRQSPSRGLEWLGRTYYRSKWYNDYAQSLKSR
ITINPDTPKNQFSLQLNSVTPEDTAVYYCARDGQL
GLDALDIWGQGTMVTVSSGGGGSDIQMTQSPSSLS
ASVGDRVTITCRASQDIRNYLNWYQQKPGKAPKLL
IYYTSRLHSGVPSRFSGSGSGTDFTFTISSLQPED
IATYYCQQGNTLPWTFGQGTKLEIKRGGGGSGGGG
SGGGGSQVQLVQSGAEVKKPGSSVKVSCKASGGTF
SGYTMNWVRQAPGQGLEWMGLINPYKGVSTYNGKF
KDRVTITADKSTSTAYMELSSLRSEDTAVYYCARS
GYYGDSDWYFDVWGQGTLVTVSSGGGGSQAGLTQP
PAVSVAPGQTASITCGRDNIGSRSVHWYQQKPGQA
PVLVVYDDSDRPAGIPERFSGSNYENTATLTISRV
EAGDEADYYCQVWGITSDHVVFGGGTKLTVLAAAD
KTHTCPPCPAPPVAGPSVFLFPPKPKDTLMISRTP
EVTCVVVDVSHEDPEVKFNWYVDGVEVHNAKTKPR
EEQYNSTYRVVSVLTVLHQDWLNGKEYKCKVSNKG
LPSSIEKTISKAKGQPREPQVCTLPPSRDELTKNQ
VSLSCAVKGFYPSDIAVEWESNGQPENNYKTTPPV
LDSDGSFFLVSKLTVDKSRWQQGNVFSCSVMHEAL
HNHYTQKSLSLSPGK
(scDbhuU3x3-43-Fc(hole)Δab)
SEQ ID NO: 8
QVQLVQSGAEVKKPGSSVKVSCKASGGTFSGYTMN
WVRQAPGQGLEWMGLINPYKGVSTYNGKFKDRVTI
TADKSTSTAYMELSSLRSEDTAVYYCARSGYYGDS
DWYFDVWGQGTLVTVSSGGGGSQAGLTQPPAVSVA
PGQTASITCGRDNIGSRSVHWYQQKPGQAPVLVVY
DDSDRPAGIPERFSGSNYENTATLTISRVEAGDEA
DYYCQVWGITSDHVVFGGGTKLTVLGGGGSGGGGS
GGGGSQVQLQQSGPGLVKPSQTLSLTCAISGDSVS
SNRAAWNWIRQSPSRGLEWLGRTYYRSKWYNDYAQ
SLKSRITINPDTPKNQFSLQLNSVTPEDTAVYYCA
RDGQLGLDALDIWGQGTMVTVSSGGGGSDIQMTQS
PSSLSASVGDRVTITCRASQDIRNYLNWYQQKPGK
APKLLIYYTSRLHSGVPSRFSGSGSGTDFTFTISS
LQPEDIATYYCQQGNTLPWTFGQGTKLEIKRAAAD
KTHTCPPCPAPPVAGPSVFLFPPKPKDTLMISRTP
EVTCVVVDVSHEDPEVKFNWYVDGVEVHNAKTKPR
EEQYNSTYRVVSVLTVLHQDWLNGKEYKCKVSNKG
LPSSIEKTISKAKGQPREPQVCTLPPSRDELTKNQ
VSLSCAVKGFYPSDIAVEWESNGQPENNYKTTPPV
LDSDGSFFLVSKLTVDKSRWQQGNVFSCSVMHEAL
HNHYTQKSLSLSPGK
(scFv3-43-Fc(knob)Δab)
SEQ ID NO: 9
QVQLQQSGPGLVKPSQTLSLTCAISGDSVSSNRAA
WNWIRQSPSRGLEWLGRTYYRSKWYNDYAQSLKSR
ITINPDTPKNQFSLQLNSVTPEDTAVYYCARDGQL
GLDALDIWGQGTMVTVSSGGGGSGGGGSGGGGSQA
GLTQPPAVSVAPGQTASITCGRDNIGSRSVHWYQQ
KPGQAPVLVVYDDSDRPAGIPERFSGSNYENTATL
TISRVEAGDEADYYCQVWGITSDHVVFGGGTKLTV
LAAADKTHTCPPCPAPPVAGPSVFLFPPKPKDTLM
ISRTPEVTCVVVDVSHEDPEVKFNWYVDGVEVHNA
KTKPREEQYNSTYRVVSVLTVLHQDWLNGKEYKCK
VSNKGLPSSIEKTISKAKGQPREPQVYTLPPCRDE
LTKNQVSLWCLVKGFYPSDIAVEWESNGQPENNYK
TTPPVLDSDGSFFLYSKLTVDKSRWQQGNVFSCSV
MHEALHNHYTQKSLSLSPGK
(Fd3-43-Fc(knob)Δab)
SEQ ID NO: 10
QVQLQQSGPGLVKPSQTLSLTCAISGDSVSSNRAA
WNWIRQSPSRGLEWLGRTYYRSKWYNDYAQSLKSR
ITINPDTPKNQFSLQLNSVTPEDTAVYYCARDGQL
GLDALDIWGQGTMVTVSSASTKGPSVFPLAPSSKS
TSGGTAALGCLVKDYFPEPVTVSWNSGALTSGVHT
FPAVLQSSGLYSLSSVVTVPSSSLGTQTYICNVNH
KPSNTKVDKKVEPKSCGTDKTHTCPPCPAPPVAGP
SVFLFPPKPKDTLMISRTPEVTCVVVDVSHEDPEV
KFNWYVDGVEVHNAKTKPREEQYNSTYRVVSVLTV
LHQDWLNGKEYKCKVSNKGLPSSIEKTISKAKGQP
REPQVYTLPPCRDELTKNQVSLWCLVKGFYPSDIA
VEWESNGQPENNYKTTPPVLDSDGSFFLYSKLTVD
KSRWQQGNVFSCSVMHEALHNHYTQKSLSLSPGK
(Vl3-43-C_{L}λ)
SEQ ID NO: 11
QAGLTQPPAVSVAPGQTASITCGRDNIGSRSVHWY
QQKPGQAPVLVVYDDSDRPAGIPERFSGSNYENTA
TLTISRVEAGDEADYYCQVWGITSDHVVFGGGTKL
TVLGQPKAAPSVTLFPPSSEELQANKATLVCLISD
FYPGAVTVAWKADSSPVKAGVETTTPSKQSNNKYA
ASSYLSLTPEQWKSHRSYSCQVTHEGSTVEKTVAP
TECS
(scDb3-43x3-43-Fc(hole)Δab)
SEQ ID NO: 12
QVQLQQSGPGLVKPSQTLSLTCAISGDSVSSNRAA
WNWIRQSPSRGLEWLGRTYYRSKWYNDYAQSLKSR
ITINPDTPKNQFSLQLNSVTPEDTAVYYCARDGQL
GLDALDIWGQGTMVTVSSGGGGSQAGLTQPPAVSV
APGQTASITCGRDNIGSRSVHWYQQKPGQAPVLVV
YDDSDRPAGIPERFSGSNYENTATLTISRVEAGDE
ADYYCQVWGITSDHVVFGGGTKLTVLGGGGSGGGG
SGGGGSQVQLQQSGPGLVKPSQTLSLTCAISGDSV
SSNRAAWNWIRQSPSRGLEWLGRTYYRSKWYNDYA
QSLKSRITINPDTPKNQFSLQLNSVTPEDTAVYYC
ARDGQLGLDALDIWGQGTMVTVSSGGGGSQAGLTQ
PPAVSVAPGQTASITCGRDNIGSRSVHWYQQKPGQ
APVLVVYDDSDRPAGIPERFSGSNYENTATLTISR
VEAGDEADYYCQVWGITSDHVVFGGGTKLTVLAAA
DKTHTCPPCPAPPVAGPSVFLFPPKPKDTLMISRT
PEVTCVVVDVSHEDPEVKFNWYVDGVEVHNAKTKP
REEQYNSTYRVVSVLTVLHQDWLNGKEYKCKVSNK
GLPSSIEKTISKAKGQPREPQVCTLPPSRDELTKN
QVSLSCAVKGFYPSDIAVEWESNGQPENNYKTTPP
VLDSDGSFFLVSKLTVDKSRWQQGNVFSCSVMHEA
LHNHYTQKSLSLSPGK
(scFvhuU3-Fc
(knob)Δab)
SEQ ID NO: 13
DIQMTQSPSSLSASVGDRVTITCRASQDIRNYLNW
YQQKPGKAPKLLIYYTSRLHSGVPSRFSGSGSGTD
FTFTISSLQPEDIATYYCQQGNTLPWTFGQGTKLE
IKRGGGGSGGGGSGGGGSQVQLVQSGAEVKKPGSS
VKVSCKASGGTFSGYTMNWVRQAPGQGLEWMGLIN
PYKGVSTYNGKFKDRVTITADKSTSTAYMELSSLR
SEDTAVYYCARSGYYGDSDWYFDVWGQGTLVTVSS
AAADKTHTCPPCPAPPVAGPSVFLFPPKPKDTLMI
SRTPEVTCVVVDVSHEDPEVKFNWYVDGVEVHNAK
TKPREEQYNSTYRVVSVLTVLHQDWLNGKEYKCKV
SNKGLPSSIEKTISKAKGQPREPQVYTLPPCRDEL
TKNQVSLWCLVKGFYPSDIAVEWESNGQPENNYKT
TPPVLDSDGSFFLYSKLTVDKSRWQQGNVFSCSVM
HEALHNHYTQKSLSLSPGK
(FdhuU3-Fc(knob)Δab)
SEQ ID NO: 14
QVQLVQSGAEVKKPGSSVKVSCKASGGTFSGYTMN
WVRQAPGQGLEWMGLINPYKGVSTYNGKFKDRVTI
TADKSTSTAYMELSSLRSEDTAVYYCARSGYYGDS
DWYFDVWGQGTLVTVSSASTKGPSVFPLAPSSKST
SGGTAALGCLVKDYFPEPVTVSWNSGALTSGVHTF
PAVLQSSGLYSLSSVVTVPSSSLGTQTYICNVNHK
PSNTKVDKKVEPKSCDKTHTCPPCPAPPVAGPSVF
LFPPKPKDTLMISRTPEVTCVVVDVSHEDPEVKFN
WYVDGVEVHNAKTKPREEQYNSTYRVVSVLTVLHQ
DWLNGKEYKCKVSNKGLPSSIEKTISKAKGQPREP
QVYTLPPCRDELTKNQVSLWCLVKGFYPSDIAVEW
ESNGQPENNYKTTPPVLDSDGSFFLYSKLTVDKSR
WQQGNVFSCSVMHEALHNHYTQKSLSLSPGK
(VLhuU3-CLK)
SEQ ID NO: 15
DIQMTQSPSSLSASVGDRVTITCRASQDIRNYLNW
YQQKPGKAPKLLIYYTSRLHSGVPSRFSGSGSGTD
FTFTISSLQPEDIATYYCQQGNTLPWTFGQGTKLE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
(Igκ-leader)
SEQ ID NO: 16
METDTLLLWVLLLWVPGSTG
(Hinge-Combination of hinge regions
of IgG1 and IgG2)
SEQ ID NO: 17
DKTHTCPPCPAPPVAG
(scDbhu225xhuU3-scFvhu225)
SEQ ID NO: 18
METDTLLLWVLLLWVPGSTGEVQLVESGGGLVQPG
GSLRLSCAASGFSLTNYGVHWVRQAPGKGLEWLGV
IWSGGNTDYNTPFTSRFTISRDNSKNTLYLQMNSL
RAEDTAVYYCARALTYYDYEFAYWGQGTTVTVSSG
GGGSDIQMTQSPSSLSASVGDRVTITCRASQDIRN
YLNWYQQKPGKAPKLLIYYTSRLHSGVPSRFSGSG
SGTDFTFTISSLQPEDIATYYCQQGNTLPWTFGQG
TKLEIKRGGGGSGGGGSGGGGSQVQLVQSGAEVKK
PGSSVKVSCKASGGTFSGYTMNWVRQAPGQGLEWM
GLINPYKGVSTYNGKFKDRVTITADKSTSTAYMEL
SSLRSEDTAVYYCARSGYYGDSDWYFDVWGQGTLV
TVSSGGGGSDIQLTQSPSFLSASVGDRVTITCRAS
QSIGTNIHWYQQKPGKAPKLLIKYASESISGVPSR
FSGSGSGTEFTLTISSLQPEDFATYYCQQNNNWPT
TFGAGTKLEIKRAAAGGSGGGGSGGGTEVQLVESG
GGLVQPGGSLRLSCAASGFSLTNYGVHWVRQAPGK
GLEWLGVIWSGGNTDYNTPFTSRFTISRDNSKNTL
YLQMNSLRAEDTAVYYCARALTYYDYEFAYWGQGT
TVTVSSGGGGSGGGGSGGGGSDIQLTQSPSFLSAS
VGDRVTITCRASQSIGTNIHWYQQKPGKAPKLLIK
YASESISGVPSRFSGSGSGTEFTLTISSLQPEDFA
TYYCQQNNNWPTTFGAGTKLEIKRSLHHHHHH
(scDbhu225xhuU3)
SEQ ID NO: 19
METDTLLLWVLLLWVPGSTGEVQLVESGGGLVQPG
GSLRLSCAASGFSLTNYGVHWVRQAPGKGLEWLGV
IWSGGNTDYNTPFTSRFTISRDNSKNTLYLQMNSL
RAEDTAVYYCARALTYYDYEFAYWGQGTTVTVSSG
GGGSDIQMTQSPSSLSASVGDRVTITCRASQDIRN
YLNWYQQKPGKAPKLLIYYTSRLHSGVPSRFSGSG
SGTDFTFTISSLQPEDIATYYCQQGNTLPWTFGQG
TKLEIKRGGGGSGGGGSGGGGSQVQLVQSGAEVKK
PGSSVKVSCKASGGTFSGYTMNWVRQAPGQGLEWM
GLINPYKGVSTYNGKFKDRVTITADKSTSTAYMEL
SSLRSEDTAVYYCARSGYYGDSDWYFDVWGQGTLV
TVSSGGGGSDIQLTQSPSFLSASVGDRVTITCRAS
QSIGTNIHWYQQKPGKAPKLLIKYASESISGVPSR
FSGSGSGTEFTLTISSLQPEDFATYYCQQNNNWPT
TFGAGTKLEIKRAAAGGSGGGGSGGGTGGGGSLHH
HHHH
(scDbCEAxhuU3-Fc(hole)Δab)
SEQ ID NO: 20
QVKLQQSGAELVRSGTSVKLSCTASGFNIKDSYMH
WLRQGPEQGLEWIGWIDPENGDTEYAPKFQGKATF
TTDTSSNTAYLQLSSLTSEDTAVYYCNEGTPTGPY
YFDYWGQGTTVTVSSGGGGSDIQMTQSPSSLSASV
GDRVTITCRASQDIRNYLNWYQQKPGKAPKLLIYY
TSRLHSGVPSRFSGSGSGTDFTFTISSLQPEDIAT
YYCQQGNTLPWTFGQGTKLEIKRGGGGSGGGGSGG
GGSQVQLVQSGAEVKKPGSSVKVSCKASGGTFSGY
TMNWVRQAPGQGLEWMGLINPYKGVSTYNGKFKDR
VTITADKSTSTAYMELSSLRSEDTAVYYCARSGYY
GDSDWYFDVWGQGTLVTVSSGGGGSDIELTQSPAI
MSASPGEKVTITCSASSSVSYMHWFQQKPGTSPKL
WIYSTSNLASGVPARFSGSGSGTSYSLTISRMEAE
DAATYYCQQRSSYPLTFGAGTKLELKRAAADKTHT
CPPCPAPPVAGPSVFLFPPKPKDTLMISRTPEVTC
VVVDVSHEDPEVKFNWYVDGVEVHNAKTKPREEQY
NSTYRVVSVLTVLHQDWLNGKEYKCKVSNKGLPSS
IEKTISKAKGQPREPQVCTLPPSRDELTKNQVSLS
CAVKGFYPSDIAVEWESNGQPENNYKTTPPVLDSD
GSFFLVSKLTVDKSRWQQGNVFSCSVMHEALHNHY
TQKSLSLSPGK
(scDbhuU3xCEA-Fc(hole)Δab)
SEQ ID NO: 21
QVQLVQSGAEVKKPGSSVKVSCKASGGTFSGYTMN
WVRQAPGQGLEWMGLINPYKGVSTYNGKFKDRVTI
TADKSTSTAYMELSSLRSEDTAVYYCARSGYYGDS
DWYFDVWGQGTLVTVSSGGGGSDIELTQSPAIMSA
SPGEKVTITCSASSSVSYMHWFQQKPGTSPKLWIY
STSNLASGVPARFSGSGSGTSYSLTISRMEAEDAA
TYYCQQRSSYPLTFGAGTKLELKRGGGGSGGGGSG
GGGSQVKLQQSGAELVRSGTSVKLSCTASGFNIKD
SYMHWLRQGPEQGLEWIGWIDPENGDTEYAPKFQG
KATFTTDTSSNTAYLQLSSLTSEDTAVYYCNEGTP
TGPYYFDYWGQGTTVTVSSGGGGSDIQMTQSPSSL
SASVGDRVTITCRASQDIRNYLNWYQQKPGKAPKL
LIYYTSRLHSGVPSRFSGSGSGTDFTFTISSLQPE
DIATYYCQQGNTLPWTFGQGTKLEIKRAAADKTHT
CPPCPAPPVAGPSVFLFPPKPKDTLMISRTPEVTC
VVVDVSHEDPEVKFNWYVDGVEVHNAKTKPREEQY
NSTYRVVSVLTVLHQDWLNGKEYKCKVSNKGLPSS
IEKTISKAKGQPREPQVCTLPPSRDELTKNQVSLS
CAVKGFYPSDIAVEWESNGQPENNYKTTPPVLDSD
GSFFLVSKLTVDKSRWQQGNVFSCSVMHEALHNHY
TQKSLSLSPGK
(scFvCEA-Fc(knob)Δab)
SEQ ID NO: 22
QVKLQQSGAELVRSGTSVKLSCTASGFNIKDSYMH
WLRQGPEQGLEWIGWIDPENGDTEYAPKFQGKATF
TTDTSSNTAYLQLSSLTSEDTAVYYCNEGTPTGPY
YFDYWGQGTTVTVSSGGGGSGGGGSGGGGSDIELT
QSPAIMSASPGEKVTITCSASSSVSYMHWFQQKPG
TSPKLWIYSTSNLASGVPARFSGSGSGTSYSLTIS
RMEAEDAATYYCQQRSSYPLTFGAGTKLELKRAAA
DKTHTCPPCPAPPVAGPSVFLFPPKPKDTLMISRT
PEVTCVVVDVSHEDPEVKFNWYVDGVEVHNAKTKP
REEQYNSTYRVVSVLTVLHQDWLNGKEYKCKVSNK
GLPSSIEKTISKAKGQPREPQVYTLPPCRDELTKN
QVSLWCLVKGFYPSDIAVEWESNGQPENNYKTTPP
VLDSDGSFFLYSKLTVDKSRWQQGNVFSCSVMHEA
LHNHYTQKSLSLSPGK
(V_{H}CEA-C_{H}1-Fc(knob)Δab)
SEQ ID NO: 23
QVKLQQSGAELVRSGTSVKLSCTASGFNIKDSYMH
WLRQGPEQGLEWIGWIDPENGDTEYAPKFQGKATF
TTDTSSNTAYLQLSSLTSEDTAVYYCNEGTPTGPY
YFDYWGQGTTVTVSSASTKGPSVFPLAPSSKSTSG
GTAALGCLVKDYFPEPVTVSWNSGALTSGVHTFPA
VLQSSGLYSLSSVVTVPSSSLGTQTYICNVNHKPS
NTKVDKKVEPKSCDKTHTCPPCPAPPVAGPSVFLF
PPKPKDTLMISRTPEVTCVVVDVSHEDPEVKFNWY
VDGVEVHNAKTKPREEQYNSTYRVVSVLTVLHQDW
LNGKEYKCKVSNKGLPSSIEKTISKAKGQPREPQV
YTLPPCRDELTKNQVSLWCLVKGFYPSDIAVEWES
NGQPENNYKTTPPVLDSDGSFFLYSKLTVDKSRWQ
QGNVFSCSVMHEALHNHYTQKSLSLSPGK
(V_{L}CEA-C_{L}κ)
SEQ ID NO: 24
DIELTQSPAIMSASPGEKVTITCSASSSVSYMHWF
QQKPGTSPKLWIYSTSNLASGVPARFSGSGSGTSY
SLTISRMEAEDAATYYCQQRSSYPLTFGAGTKLEL
KRRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
(scDbCEAxCEA-Fc(hole)Δab)
SEQ ID NO: 25
QVKLQQSGAELVRSGTSVKLSCTASGFNIKDSYMH
WLRQGPEQGLEWIGWIDPENGDTEYAPKFQGKATF
TTDTSSNTAYLQLSSLTSEDTAVYYCNEGTPTGPY
YFDYWGQGTTVTVSSGGGGSDIELTQSPAIMSASP
GEKVTITCSASSSVSYMHWFQQKPGTSPKLWIYST
SNLASGVPARFSGSGSGTSYSLTISRMEAEDAATY
YCQQRSSYPLTFGAGTKLELKRGGGGSGGGGSGGG
GSQVKLQQSGAELVRSGTSVKLSCTASGFNIKDSY
MHWLRQGPEQGLEWIGWIDPENGDTEYAPKFQGKA
TFTTDTSSNTAYLQLSSLTSEDTAVYYCNEGTPTG
PYYFDYWGQGTTVTVSSGGGGSDIELTQSPAIMSA
SPGEKVTITCSASSSVSYMHWFQQKPGTSPKLWIY
STSNLASGVPARFSGSGSGTSYSLTISRMEAEDAA
TYYCQQRSSYPLTFGAGTKLELKRAAADKTHTCPP
CPAPPVAGPSVFLFPPKPKDTLMISRTPEVTCVVV
DVSHEDPEVKFNWYVDGVEVHNAKTKPREEQYNST
YRVVSVLTVLHQDWLNGKEYKCKVSNKGLPSSIEK
TISKAKGQPREPQVCTLPPSRDELTKNQVSLSCAV
KGFYPSDIAVEWESNGQPENNYKTTPPVLDSDGSF
FLVSKLTVDKSRWQQGNVFSCSVMHEALHNHYTQK
SLSLSPGK
(scDbhu225xhuU3-Fc
(hole)Δab)
SEQ ID NO: 26
EVQLVESGGGLVQPGGSLRLSCAASGFSLTNYGVH
WVRQAPGKGLEWLGVIWSGGNTDYNTPFTSRFTIS
RDNSKNTLYLQMNSLRAEDTAVYYCARALTYYDYE
FAYWGQGTTVTVSSGGGGSDIQMTQSPSSLSASVG
DRVTITCRASQDIRNYLNWYQQKPGKAPKLLIYYT
SRLHSGVPSRFSGSGSGTDFTFTISSLQPEDIATY
YCQQGNTLPWTFGQGTKLEIKRGGGGSGGGGSGGG
GSQVQLVQSGAEVKKPGSSVKVSCKASGGTFSGYT
MNWVRQAPGQGLEWMGLINPYKGVSTYNGKFKDRV
TITADKSTSTAYMELSSLRSEDTAVYYCARSGYYG
DSDWYFDVWGQGTLVTVSSGGGGSDIQLTQSPSFL
SASVGDRVTITCRASQSIGTNIHWYQQKPGKAPKL
LIKYASESISGVPSRFSGSGSGTEFTLTISSLQPE
DFATYYCQQNNNWPTTFGAGTKLEIKRAAADKTHT
CPPCPAPPVAGPSVFLFPPKPKDTLMISRTPEVTC
VVVDVSHEDPEVKFNWYVDGVEVHNAKTKPREEQY
NSTYRVVSVLTVLHQDWLNGKEYKCKVSNKGLPSS
IEKTISKAKGQPREPQVCTLPPSRDELTKNQVSLS
CAVKGFYPSDIAVEWESNGQPENNYKTTPPVLDSD
GSFFLVSKLTVDKSRWQQGNVFSCSVMHEALHNHY
TQKSLSLSPGK
(scDbhuU3xhu225-Fc
(hole)Δab)
SEQ ID NO: 27
QVQLVQSGAEVKKPGSSVKVSCKASGGTFSGYTMN
WVRQAPGQGLEWMGLINPYKGVSTYNGKFKDRVTI
TADKSTSTAYMELSSLRSEDTAVYYCARSGYYGDS
DWYFDVWGQGTLVTVSSGGGGSDIQLTQSPSFLSA
SVGDRVTITCRASQSIGTNIHWYQQKPGKAPKLLI
KYASESISGVPSRFSGSGSGTEFTLTISSLQPEDF
ATYYCQQNNNWPTTFGAGTKLEIKRGGGGSGGGGS
GGGGSEVQLVESGGGLVQPGGSLRLSCAASGFSLT
NYGVHWVRQAPGKGLEWLGVIWSGGNTDYNTPFTS
RFTISRDNSKNTLYLQMNSLRAEDTAVYYCARALT
YYDYEFAYWGQGTTVTVSSGGGGSDIQMTQSPSSL
SASVGDRVTITCRASQDIRNYLNWYQQKPGKAPKL
LIYYTSRLHSGVPSRFSGSGSGTDFTFTISSLQPE
DIATYYCQQGNTLPWTFGQGTKLEIKRAAADKTHT
CPPCPAPPVAGPSVFLFPPKPKDTLMISRTPEVTC
VVVDVSHEDPEVKFNWYVDGVEVHNAKTKPREEQY
NSTYRVVSVLTVLHQDWLNGKEYKCKVSNKGLPSS
IEKTISKAKGQPREPQVCTLPPSRDELTKNQVSLS
CAVKGFYPSDIAVEWESNGQPENNYKTTPPVLDSD
GSFFLVSKLTVDKSRWQQGNVFSCSVMHEALHNHY
TQKSLSLSPGK
(scFvhu225-Fc
(knob)Δab)
SEQ ID NO: 28
EVQLVESGGGLVQPGGSLRLSCAASGFSLTNYGVH
WVRQAPGKGLEWLGVIWSGGNTDYNTPFTSRFTIS
RDNSKNTLYLQMNSLRAEDTAVYYCARALTYYDYE
FAYWGQGTTVTVSSGGGGSGGGGSGGGGSDIQLTQ
SPSFLSASVGDRVTITCRASQSIGTNIHWYQQKPG
KAPKLLIKYASESISGVPSRFSGSGSGTEFTLTIS
SLQPEDFATYYCQQNNNWPTTFGAGTKLEIKRAAA
DKTHTCPPCPAPPVAGPSVFLFPPKPKDTLMISRT
PEVTCVVVDVSHEDPEVKFNWYVDGVEVHNAKTKP
REEQYNSTYRVVSVLTVLHQDWLNGKEYKCKVSNK
GLPSSIEKTISKAKGQPREPQVYTLPPCRDELTKN
QVSLWCLVKGFYPSDIAVEWESNGQPENNYKTTPP
VLDSDGSFFLYSKLTVDKSRWQQGNVFSCSVMHEA
LHNHYTQKSLSLSPGK
(Fdhu225-Fc
(knob)Δab)
SEQ ID NO: 29
EVQLVESGGGLVQPGGSLRLSCAASGFSLTNYGVH
WVRQAPGKGLEWLGVIWSGGNTDYNTPFTSRFTIS
RDNSKNTLYLQMNSLRAEDTAVYYCARALTYYDYE
FAYWGQGTTVTVSSASTKGPSVFPLAPSSKSTSGG
TAALGCLVKDYFPEPVTVSWNSGALTSGVHTFPAV
LQSSGLYSLSSVVTVPSSSLGTQTYICNVNHKPSN
TKVDKKVEPKSCGTDKTHTCPPCPAPPVAGPSVFL
FPPKPKDTLMISRTPEVTCVVVDVSHEDPEVKFNW
YVDGVEVHNAKTKPREEQYNSTYRVVSVLTVLHQD
WLNGKEYKCKVSNKGLPSSIEKTISKAKGQPREPQ
VYTLPPCRDELTKNQVSLWCLVKGFYPSDIAVEWE
SNGQPENNYKTTPPVLDSDGSFFLYSKLTVDKSRW
QQGNVFSCSVMHEALHNHYTQKSLSLSPGK
(V_{L}hu225-C_{L}κ)
SEQ ID NO: 30
DIQLTQSPSFLSASVGDRVTITCRASQSIGTNIHW
YQQKPGKAPKLLIKYASESISGVPSRFSGSGSGTE
FTLTISSLQPEDFATYYCQQNNNWPTTFGAGTKLE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
(scDbhu225xhu225-Fc
(hole)Δab)
SEQ ID NO: 31
EVQLVESGGGLVQPGGSLRLSCAASGFSLTNYGVH
WVRQAPGKGLEWLGVIWSGGNTDYNTPFTSRFTIS
RDNSKNTLYLQMNSLRAEDTAVYYCARALTYYDYE
FAYWGQGTTVTVSSGGGGSDIQLTQSPSFLSASVG
DRVTITCRASQSIGTNIHWYQQKPGKAPKLLIKYA
SESISGVPSRFSGSGSGTEFTLTISSLQPEDFATY
YCQQNNNWPTTFGAGTKLEIKRGGGGSGGGGSGGG
GSEVQLVESGGGLVQPGGSLRLSCAASGFSLTNYG
VHWVRQAPGKGLEWLGVIWSGGNTDYNTPFTSRFT
ISRDNSKNTLYLQMNSLRAEDTAVYYCARALTYYD
YEFAYWGQGTTVTVSSGGGGSDIQLTQSPSFLSAS
VGDRVTITCRASQSIGTNIHWYQQKPGKAPKLLIK
YASESISGVPSRFSGSGSGTEFTLTISSLQPEDFA
TYYCQQNNNWPTTFGAGTKLEIKRAAADKTHTCPP
CPAPPVAGPSVFLFPPKPKDTLMISRTPEVTCVVV
DVSHEDPEVKFNWYVDGVEVHNAKTKPREEQYNST
YRVVSVLTVLHQDWLNGKEYKCKVSNKGLPSSIEK
TISKAKGQPREPQVCTLPPSRDELTKNQVSLSCAV
KGFYPSDIAVEWESNGQPENNYKTTPPVLDSDGSF
FLVSKLTVDKSRWQQGNVFSCSVMHEALHNHYTQK
SLSLSPGK
(scDbhu225x3-43-Fc
(hole)Δab)
SEQ ID NO: 32
EVQLVESGGGLVQPGGSLRLSCAASGFSLTNYGVH
WVRQAPGKGLEWLGVIWSGGNTDYNTPFTSRFTIS
RDNSKNTLYLQMNSLRAEDTAVYYCARALTYYDYE
FAYWGQGTTVTVSSGGGGSQAGLTQPPAVSVAPGQ
TASITCGRDNIGSRSVHWYQQKPGQAPVLVVYDDS
DRPAGIPERFSGSNYENTATLTISRVEAGDEADYY
CQVWGITSDHVVFGGGTKLTVLGGGGSGGGGSGGG
GSQVQLQQSGPGLVKPSQTLSLTCAISGDSVSSNR
AAWNWIRQSPSRGLEWLGRTYYRSKWYNDYAQSLK
SRITINPDTPKNQFSLQLNSVTPEDTAVYYCARDG
QLGLDALDIWGQGTMVTVSSGGGGSDIQLTQSPSF
LSASVGDRVTITCRASQSIGTNIHWYQQKPGKAPK
LLIKYASESISGVPSRFSGSGSGTEFTLTISSLQP
EDFATYYCQQNNNWPTTFGAGTKLEIKRAAADKTH
TCPPCPAPPVAGPSVFLFPPKPKDTLMISRTPEVT
CVVVDVSHEDPEVKFNWYVDGVEVHNAKTKPREEQ
YNSTYRVVSVLTVLHQDWLNGKEYKCKVSNKGLPS
SIEKTISKAKGQPREPQVCTLPPSRDELTKNQVSL
SCAVKGFYPSDIAVEWESNGQPENNYKTTPPVLDS
DGSFFLVSKLTVDKSRWQQGNVFSCSVMHEALHNH
YTQKSLSLSPGK
(scDbFAPxCD3-Fc
(hole)Δab)
SEQ ID NO: 33
QVQLVQSGAEVKKPGASVKVSCKASGYTFTENIIH
WVRQAPGQGLEWMGWFHPGSGSIKYNEKFKDRVTM
TADTSTSTVYMELSSLRSEDTAVYYCARHGGTGRG
AMDYWGQGTLVTVSSGGGGSDIQMTQSPSSLSASV
GDRVTITCRASQDIRNYLNWYQQKPGKAPKLLIYY
TSRLHSGVPSRFSGSGSGTDFTFTISSLQPEDIAT
YYCQQGNTLPWTFGQGTKLEIKRGGGGSGGGGSGG
GGSQVQLVQSGAEVKKPGSSVKVSCKASGGTFSGY
TMNWVRQAPGQGLEWMGLINPYKGVSTYNGKFKDR
VTITADKSTSTAYMELSSLRSEDTAVYYCARSGYY
GDSDWYFDVWGQGTLVTVSSGGGGSDIQMTQSPSS
LSASVGDRVTITCRASKSVSTSAYSYMHWYQQKPG
KAPKLLIYLASNLESGVPSRFSGSGSGTDFTLTIS
SLQPEDFATYYCQHSRELPYTFGQGTKLEIKRAAA
DKTHTCPPCPAPPVAGPSVFLFPPKPKDTLMISRT
PEVTCVVVDVSHEDPEVKFNWYVDGVEVHNAKTKP
REEQYNSTYRVVSVLTVLHQDWLNGKEYKCKVSNK
GLPSSIEKTISKAKGQPREPQVCTLPPSRDELTKN
QVSLSCAVKGFYPSDIAVEWESNGQPENNYKTTPP
VLDSDGSFFLVSKLTVDKSRWQQGNVFSCSVMHEA
LHNHYTQKSLSLSPGK
(scDbCD3xFAP-Fc
(hole)Δab)
SEQ ID NO: 34
QVQLVQSGAEVKKPGSSVKVSCKASGGTFSGYTMN
WVRQAPGQGLEWMGLINPYKGVSTYNGKFKDRVTI
TADKSTSTAYMELSSLRSEDTAVYYCARSGYYGDS
DWYFDVWGQGTLVTVSSGGGGSDIQMTQSPSSLSA
SVGDRVTITCRASKSVSTSAYSYMHWYQQKPGKAP
KLLIYLASNLESGVPSRFSGSGSGTDFTLTISSLQ
PEDFATYYCQHSRELPYTFGQGTKLEIKRGGGGSG
GGGSQVQLVQSGAEVKKPGASVKVSCKASGYTFTE
NIIHWVRQAPGQGLEWMGWFHPGSGSIKYNEKFKD
RVTMTADTSTSTVYMELSSLRSEDTAVYYCARHGG
TGRGAMDYWGQGTLVTVGGGGSDIQMTQSPSSLSA
SVGDRVTITCRASQDIRNYLNWYQQKPGKAPKLLI
YYTSRLHSGVPSRFSGSGSGTDFTFTISSLQPEDI
ATYYCQQGNTLPWTFGQGTKLEIKRAAADKTHTCP
PCPAPPVAGPSVFLFPPKPKDTLMISRTPEVTCVV
VDVSHEDPEVKFNWYVDGVEVHNAKTKPREEQYNS
TYRVVSVLTVLHQDWLNGKEYKCKVSNKGLPSSIE
KTISKAKGQPREPQVCTLPPSRDELTKNQVSLSCA
VKGFYPSDIAVEWESNGQPENNYKTTPPVLDSDGS
FFLVSKLTVDKSRWQQGNVFSCSVMHEALHNHYTQ
KSLSLSPGK
(scFvFAP-Fc
(knob)Δab)
SEQ ID NO: 35
QVQLVQSGAEVKKPGASVKVSCKASGYTFTENIIH
WVRQAPGQGLEWMGWFHPGSGSIKYNEKFKDRVTM
TADTSTSTVYMELSSLRSEDTAVYYCARHGGTGRG
AMDYWGQGTLVTVSSGGGGSGGGGSGGGGSDIQMT
QSPSSLSASVGDRVTITCRASKSVSTSAYSYMHWY
QQKPGKAPKLLIYLASNLESGVPSRFSGSGSGTDF
TLTISSLQPEDFATYYCQHSRELPYTFGQGTKLEI
KRAAADKTHTCPPCPAPPVAGPSVFLFPPKPKDTL
MISRTPEVTCVVVDVSHEDPEVKFNWYVDGVEVHN
AKTKPREEQYNSTYRVVSVLTVLHQDWLNGKEYKC
KVSNKGLPSSIEKTISKAKGQPREPQVYTLPPCRD
ELTKNQVSLWCLVKGFYPSDIAVEWESNGQPENNY
KTTPPVLDSDGSFFLYSKLTVDKSRWQQGNVFSCS
VMHEALHNHYTQKSLSLSPGK
(V_{H}FAP-C_{H}1-Fc
(knob)Δab)
SEQ ID NO: 36
QVQLVQSGAEVKKPGASVKVSCKASGYTFTENIIH
WVRQAPGQGLEWMGWFHPGSGSIKYNEKFKDRVTM
TADTSTSTVYMELSSLRSEDTAVYYCARHGGTGRG
AMDYWGQGTLVTVSSASTKGPSVFPLAPSSKSTSG
GTAALGCLVKDYFPEPVTVSWNSGALTSGVHTFPA
VLQSSGLYSLSSVVTVPSSSLGTQTYICNVNHKPS
NTKVDKKVEPKSCDKTHTCPPCPAPPVAGPSVFLF
PPKPKDTLMISRTPEVTCVVVDVSHEDPEVKFNWY
VDGVEVHNAKTKPREEQYNSTYRVVSVLTVLHQDW
LNGKEYKCKVSNKGLPSSIEKTISKAKGQPREPQV
YTLPPCRDELTKNQVSLWCLVKGFYPSDIAVEWES
NGQPENNYKTTPPVLDSDGSFFLYSKLTVDKSRWQ
QGNVFSCSVMHEALHNHYTQKSLSLSPGK
(V_{L}FAP-C_{L}κ)
SEQ ID NO: 37
DIQMTQSPSSLSASVGDRVTITCRASKSVSTSAYS
YMHWYQQKPGKAPKLLIYLASNLESGVPSRFSGSG
SGTDFTLTISSLQPEDFATYYCQHSRELPYTFGQG
TKLEIKRRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC
(scDbFAPxFAP-Fc
(hole)Δab)
SEQ ID NO: 38
QVQLVQSGAEVKKPGASVKVSCKASGYTFTENIIH
WVRQAPGQGLEWMGWFHPGSGSIKYNEKFKDRVTM
TADTSTSTVYMELSSLRSEDTAVYYCARHGGTGRG
AMDYWGQGTLVTVSSGGGGSDIQMTQSPSSLSASV
GDRVTITCRASKSVSTSAYSYMHWYQQKPGKAPKL
LIYLASNLESGVPSRFSGSGSGTDFTLTISSLQPE
DFATYYCQHSRELPYTFGQGTKLEIKRGGGGSGGG
GSQVQLVQSGAEVKKPGASVKVSCKASGYTFTENI
IHWVRQAPGQGLEWMGWFHPGSGSIKYNEKFKDRV
TMTADTSTSTVYMELSSLRSEDTAVYYCARHGGTG
RGAMDYWGQGTLVTVGGGGSDIQMTQSPSSLSASV
GDRVTITCRASKSVSTSAYSYMHWYQQKPGKAPKL
LIYLASNLESGVPSRFSGSGSGTDFTLTISSLQPE
DFATYYCQHSRELPYTFGQGTKLEIKRAAADKTHT
CPPCPAPPVAGPSVFLFPPKPKDTLMISRTPEVTC
VVVDVSHEDPEVKFNWYVDGVEVHNAKTKPREEQY
NSTYRVVSVLTVLHQDWLNGKEYKCKVSNKGLPSS
IEKTISKAKGQPREPQVCTLPPSRDELTKNQVSLS
CAVKGFYPSDIAVEWESNGQPENNYKTTPPVLDSD
GSFFLVSKLTVDKSRWQQGNVFSCSVMHEALHNHY
TQKSLSLSPGK

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. In particular, any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The term “binding” according to the invention preferably relates to a specific binding. “Specific binding” means that a binding protein (e.g. an antibody) binds stronger to a target such as an epitope for which it is specific compared to the binding to another target. A binding protein binds stronger to a first target compared to a second target if it binds to the first target with a dissociation constant (K_{d}) which is lower than the dissociation constant for the second target. Preferably the dissociation constant (K_{d}) for the target to which the binding protein binds specifically is more than 10-fold, preferably more than 20-fold, more preferably more than 50-fold, even more preferably more than 100-fold, 200-fold, 500-fold or 1000-fold lower than the dissociation constant (K_{d}) for the target to which the binding protein does not bind specifically.

Likewise, as used herein, the term “binding domain” refers to a protein domain capable of binding to an antigen.

As used herein, the terms “linker” and “peptide linker” refer to an amino acid sequence, i.e. polypeptide, which sterically separates two parts within the engineered polypeptides of the present invention. Typically, such peptide linker consists of between 1 and 100, preferably 3 to 50 more preferably 5 to 20 amino acids. Thus, such peptide linkers have a minimum length of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids, and a maximum length of at least 100, 95, 90, 85, 80, 75, 70, 65, 60, 55, 50, 45, 40, 35, 34, 33, 32, 31, 30, 29, 28, 27, 26, 25, 24, 23, 22, 21, 20, 19, 18, 17, 16, or 15 amino acids or less. Preferred linker lengths are between 3 and 15 amino acids. Peptide linkers may also provide flexibility among the two parts that are linked together. Such flexibility is generally increased, if the amino acids are small. Accordingly, flexible peptide linkers comprise an increased content of small amino acids, in particular of glycins and/or alanines, and/or hydrophilic amino acids such as serines, threonines, asparagines and glutamines. Preferably, more than 20%, 30%, 40%, 50%, 60% or more of the amino acids of the peptide linker are small amino acids. Preferred peptide linkers have the sequence GGGGS (SEQ ID NO:1), [G₄S]₃(SEQ ID NO:2), AAAGGSGGGGSGGGT (SEQ ID NO:3), or AAA (SEQ ID NO:4).

The term “variable chain” if referring to an antibody-like structure refers to the variable regions of both light (V_{L}) and heavy (V_{H}) chains that determine binding recognition and specificity to the antigen. The term “variable domain” may also refer to the variable domain or region of a TCR α- and a TCR β-chain.

As used herein, the term “TCR” or “T cell receptor” denotes a molecule found on the surface of T cells. The TCR is composed of two separate peptide chains, which are produced from the independent T cell receptor alpha and beta (TCRα and TCRβ) genes. These chains are called α- and β-chains. Each of the TCR α- and the TCR β-chain has a variable and a constant domain. Each variable domain carries three CDRs for binding to an antigen.

The term “constant domain of a TCR” and like terms used herein denote the constant region or domain of TCR α- and a TCR β-chain.

The term “antigen” or “target antigen” as used herein refers to a molecule or a portion of a molecule that is capable of being bound by an antibody, an antibody-like binding protein or a T cell receptor. The term further refers to a molecule or a portion of a molecule that is capable of being used in an animal to produce antibodies that are capable of binding to an epitope of that antigen. A target antigen may have one or more epitopes.

The term “Fab” means antigen-binding fragment and denotes an antibody fragment that binds to antigens. It is composed of one constant and one variable domain of each of the heavy and the light chain. It usually has a molecular weight of about 50,000 and about half of the N-terminal side of H chain and the entire L chain, among fragments obtained by treating IgG with a protease, papaine, are bound together through a disulfide bond.

The term “scFv” means single-chain variable fragment and denotes a fusion protein of the variable regions of the heavy (VH) and light chains (VL) of immunoglobulins, connected with a short linker peptide of ten to about 25 amino acids.

As used herein, the term “variable heavy chain domain” or “variable light chain domain”, also referred to as the Fv region or Fv, refers to the part of an immunglobulin heavy and light chain, respectively, that comprises variable loops of β-strands that are responsible for binding to the antigen. These loops are also referred to as the complementarity determining regions (CDRs).

As used herein, the term “diabody” refers to a bivalent molecule that can bind to two antigens, either of the same type (monospecific) or to different antigens (bispecific). Diabodies are described e.g. in Holliger et al. (1993) Proc. Natl. Acad. Sci. U.S.A. 90(14), 6444-6448, the contents of which are hereby incorporated by reference.

As used herein, the term “single-chain diabody (scDb)” refers to derivatives of diabodies in which the four variable domains of one or two antibodies are connected by three linkers.

The term “binding” according to the present invention preferably relates to a specific binding. “Specific binding” means that a binding protein (e.g. an antibody) binds stronger to a target such as an epitope for which it is specific compared to the binding to another target. A binding protein binds stronger to a first target compared to a second target if it binds to the first target with a dissociation constant (K_{d}) which is lower than the dissociation constant for the second target. Preferably the dissociation constant (K_{d}) for the target to which the binding protein binds specifically is more than 10-fold, preferably more than 20-fold, more preferably more than 50-fold, even more preferably more than 100-fold, 200-fold, 500-fold or 1000-fold lower than the dissociation constant (K_{d}) for the target to which the binding protein does not bind specifically.

As used herein, the term “K_{d}” (measured in “mol/L”, sometimes abbreviated as “M”) is intended to refer to the dissociation equilibrium constant of the particular interaction between a binding protein (e.g. an antibody or fragment thereof) and a target molecule (e.g. an antigen or epitope thereof). Methods for determining binding affinities of compounds, i.e. for determining the dissociation constant K_{d}, are known to a person of ordinary skill in the art and can be selected for instance from the following methods known in the art: Surface Plasmon Resonance (SPR) based technology, preferably using a Biacore platform, Bio-layer interferometry (BLI), quartz crystal microbalance (QCM), enzyme-linked immunosorbent assay (ELISA), flow cytometry, isothermal titration calorimetry (ITC), analytical ultracentrifugation, radioimmunoassay (RIA or IRMA) and enhanced chemiluminescence (ECL). In the context of the present application, the “K_{d}” value is determined by surface plasmon resonance spectroscopy (Biacore™) or by quartz crystal microbalance (QCM) at room temperature (25° C.).

As used herein, the term “single-chain dual valence antigen binding polypeptide (scDVAP)” refers to an antigen binding peptide having one chain of amino acids and possessing two antigen binding sites.

The term “antigen binding protein”, “antigen binding peptide” and “antigen binding polypeptide” as used herein, refers to any molecule or part of a molecule that can specifically bind to a target molecule or target epitope. Preferred binding proteins in the context of the present application are (a) antibodies or antigen-binding fragments thereof; (b) oligonucleotides; (c) antibody-like proteins; (d) peptidomimetics; or (e) the variable domain of a TCR α- or a TCR β-chain.

The term “antigen-binding fragment”, such as an antigen-binding fragment of an antibody (or simply “binding portion”), as used herein, refers to one or more fragments of an antibody or T cell receptor (TCR) that retain the ability to specifically bind to an antigen. It has been shown that the antigen-binding function of an antibody can be performed by fragments of a full-length antibody. Examples of binding fragments encompassed within the term “antigen-binding portion” of an antibody include (i) Fab fragments, monovalent fragments consisting of the VL, VH, CL and CH domains; (ii) F(ab′)₂ fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341: 544-546), which consist of a VH domain or a VL domain, a VHH, a Nanobody, or a variable domain of an IgNAR; (vi) isolated complementarity determining regions (CDR), and (vii) combinations of two or more isolated CDRs which may optionally be joined by a synthetic peptide linker. Furthermore, although the two domains of the Fv fragment, VL and VH, are coded for by separate genes, they can be joined, using recombinant methods, by a synthetic peptide linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding fragment” of an antibody. A further example is a binding-domain immunoglobulin fusion protein comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. The binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies. Further examples of “antigen-binding fragments” are so-called microantibodies, which are derived from single CDRs. For example, Heap et al., 2005(J Gen Virol, 86,1791-1800), describe a 17 amino acid residue microantibody derived from the heavy chain CDR3 of an antibody directed against the gp120 envelope glycoprotein of HIV-1. Other examples include small antibody mimetics comprising two or more CDR regions that are fused to each other, preferably by cognate framework regions. Such a small antibody mimetic comprising V_{H} CDR1 and V_{L} CDR3 linked by the cognate V_{H} FR2 has been described by Qiu et al. (Nat Biotechnol. 2007, 25, 921-929).

Antibodies and antigen-binding fragments thereof suitable for use in the present invention include, but are not limited to, polyclonal, monoclonal, monovalent, bispecific, heteroconjugate, multispecific, recombinant, heterologous, heterohybrid, chimeric, humanized (in particular CDR-grafted), deimmunized, or human antibodies, Fab fragments, Fab′ fragments, F(ab′)₂ fragments, fragments produced by a Fab expression library, Fd, Fv, disulfide-linked FVS (dsFv), single chain antibodies (e.g. scFv), diabodies or tetrabodies (Holliger P. et al. (1993) Proc. Natl. Acad. Sci. U.S.A. 90(14), 6444-6448), nanobodies (also known as single domain antibodies), anti-idiotypic (anti-Id) antibodies (including, e.g., anti-Id antibodies to antibodies of the invention), and epitope-binding fragments of any of the above.

As used herein, the term “trigger molecule on an immune effector cell” refers to molecules coupled to a receptor molecule of the immune system such as pattern recognition receptors (PRRs), Toll-like receptors (TLRs), killer activated and killer inhibitor receptors (KARs and KIRs), complement receptors, Fc receptors, B cell receptors and T cell receptors. Binding to these receptors causes a response in the immune system via the trigger molecules. A trigger molecule on an immune effector cell is preferably selected from the group consisting of CD2, CD3, CD16, CD44, CD64, CD69, CD89, Mel14, Ly-6.2C, TCR-complex, Vy9V52 TCR, or NKG2D. A preferred trigger molecule is CD3.

The term “dimerization domain” refers to a domain capable of forming a dimer of two peptide or protein chains, wherein at least one dimerization domain is present on the first chain and at least a second dimerization domain is present on the second chain. A dimerization domain can be selected from the group consisting of an Fc region, a heterodimerizing Fc region, C_{H}1, C_{L}, the second heavy chain constant domain (C_{H}2) of IgE and IgM (EHD2, MHD2), modified EHD2, the last heavy chain constant domain (C_{H}3 or C_{H}4) of IgG, IgD, IgA, IgM, or IgE and heterodimerizing derivatives thereof, and the constant domains C-α and C-β of a T-cell receptor. Depending on the respective dimerization domain, the C-terminus and N-terminus of the dimerization domain may vary. If the dimerization domain is derived from a naturally occurring protein, e.g. an immunoglobulin, the dimerization domain is, preferably, directly linked to the variable domain in the sense of the present invention, i.e. linked without a peptide linker, if there are no non-naturally occurring amino acids at its C- or N-terminus.

The term “Fc chain” or “Fc part” as used herein refers to a structure which can form a homodimer or heterodimer, and binds to the respective effector molecules preferably with either increased or reduced affinity, thus altering the effector function, e.g. ADCC, CMC, or FcRn-mediated recycling. There are different IgG variants with altered interaction for human FcγRIIIa (CD16) described in literature (Presta et al., 2008, Curr Opin Immunol. 20: 460-470), e.g. IgG1-DE (S239D, I332E) resulting in 10-fold increased ADCC, or IgG1-DEL (S239D, I332E, A330L) resulting in 100-fold increased ADCC. Besides increasing the effector function, there are also Fc parts with reduced effector function described in the literature. For the IgG1-P329G LALA variant (L234A, L235A, P329G) almost complete abolished interaction with the whole Fcγ receptor family was reported, resulting in effector silent molecules (Schlothauer et al., 2016, Protein Eng Des Sel. 29; 457-466). In addition, reduced binding to FcγRI, which was described for the IgG-Δab variant (E233P, L234V, L235A, Δ236G, A327G, A330S, P331S) also resulted in reduced effector function (Armour et al., 1999; Eur J Immunol. 29: 2612-2624) (also described in Strohl et al., 2009; Curr Opin Biotechnol; 20: 685-691). Besides altering binding to receptors of immune cells (e.g. human FcγRIIIa), also binding to FcRn can be altered by introducing substitutions in the Fc part. Due to increased or reduced binding to the FcRn molecule, half-life of the Fc-containing molecule is affected, e.g. IgG1-YTE (M252Y, S254T, T256E) resulting in 3-4 fold increased terminal half-life of the protein, or IgG1-QL (T250Q, M428L) resulting in 2.5-fold increased terminal half-life (Presto et al., 2008; Strohl et al., 2009).

The term “heterodimerizing Fc” part relates to variants of a Fc part, which are able to form heterodimers. Besides the knob-into-hole technology there are other variants of the Fc part described in literature for the generation of heterodimeric Fc parts (Krah et al., 2017, N. Biotechnol. 39: 167-173; Ha et al., 2016, Front Immuno. 7: 394; Mimoto et al., 2016, Curr Pharm Biotechnol. 17: 1298-1314; Brinkmann & Kontermann, 2017, MAbs 9: 182-212). The “Knob-into-Hole” or also called “Knobs-into-Holes” technology refers to mutations Y349C, T366S, L368A and Y407V (Hole) and S354C and T366W (Knob) both in the CH3-CH3 interface to promote heteromultimer formation and has been described in U.S. Pat. Nos. 5,731,168 and 8,216,805, notably, both of which are herein incorporated by reference.

### EMBODIMENTS

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect, the present invention provides a trivalent binding molecule comprising:
(A) a first polypeptide comprising a single-chain dual valence antigen binding polypeptide (scDVAP), wherein the scDVAP comprises a first binding domain comprising a first variable chain (VC1) and a second variable chain (VC2), and a second binding domain comprising a third variable chain (VC3) and a fourth variable chain (VC4), wherein VC1 and VC2 together form a first antigen binding site, and VC3 and VC4 together form a second antigen binding site, wherein
   (i) VC1 and VC4 are connected by a first peptide linker (L1), VC4 and VC3 are connected by a second peptide linker (L2), and VC3 and VC2 are connected by a third peptide linker (L3), or
   (ii) wherein VC4 and VC1 are connected by a first peptide linker (L1), VC1 and VC2 are connected by a second peptide linker (L2), and VC2 and VC3 are connected by a third peptide linker (L3),
(B) a second polypeptide comprising a third binding domain comprising a fifth variable chain (VC5) and a sixth variable chain (VC6), wherein VC5 and VC6 together form a third antigen binding site, wherein
   (a) two of the binding sites of the trivalent binding molecule specifically bind to the same or different antigens which is not a trigger molecule on an immune effector cell,
   (b) only one of the binding sites of the trivalent binding molecule is directed against a trigger molecule on an immune effector cell, and
   (c) the first and second polypeptide are interconnected.

According to the present invention, each binding domain comprises a pair of variable chains (VC), which together form an antigen binding site. The variable chains can each be selected from the group consisting of an α-chain variable domain, β-chain variable domain, γ-chain variable domain, δ-chain variable domain, variable light chain domain and variable heavy chain domain and any combinations thereof. The trivalent binding molecule of the present invention may thus have T-cell receptor (TCR) characteristics if comprising α-chain and β-chain variable domains or γ-chain and δ-chain variable domains, or may have antibody characteristics if comprising variable light and heavy chain domains. The trivalent binding molecule of the present invention may also have both, TCR characteristics and antibody characteristics if one or two of the pairs of variable chains have TCR characteristics and the remaining pair(s) of variable chains have antibody characteristics. There are six variable chains in the trivalent binding molecule according to the present invention, four of which form part of the first polypeptide and two of which form part of the second polypeptide. Within each polypeptide, the variable chains are interconnected by means of peptide linkers. The peptide linkers may consist of between 1 and 100, preferably 3 to 50, 5 to 20, 5, 6, 7, 8, 9, 10, 11, 12, 13, 14, 15, 16, 17, 18, 19 and 20 amino acids. According to a preferred embodiment, the peptide linker(s) consist(s) of between 5 and 18 amino acids. The linkers can be the same or different. Preferred peptide linkers can be selected from the group consisting of SEQ ID NO:1 to 4, but are not limited thereto.

According to the present invention, the variable chains of the first polypeptide VC1 and VC4 are connected by a first peptide linker (L1), VC4 and VC3 are connected by a second peptide linker (L2), and VC3 and VC2 are connected by a third peptide linker (L3). Alternatively, VC4 and VC1 are connected by a first peptide linker (L1), VC1 and VC2 are connected by a second peptide linker (L2), and VC2 and VC3 are connected by a third peptide linker (L3). The variable chains VC5 and VC6 of the second polypeptide can optionally be connected by a fourth peptide linker (L4). Interconnecting the four variable chains VC1 to VC4 by means of peptide linkers gives rise to a single-chain polypeptide having dual valence antigen binding capabilities due to the four variable chains forming the first and second binding domain. Such polypeptide is referred to herein as single-chain dual valence antigen binding polypeptide (scDVAP).

Thus, in the trivalent binding molecule of the present invention, the first polypeptide has the structure of:
(i) VC1-L1-VC4-L2-VC3-L3-VC2; or
(ii) VC4-L1-VC1-L2-VC2-L3-VC3.

The desired pairing of variable domains, i.e. that VC1 and VC2 together form a first antigen binding site, and VC3 and VC4 together form a second antigen binding is facilitated by the choice of the linker length. To avoid the pairing of adjacent VC1 and VC4 the linker L1 should be too short to allow interaction between VC1 and VC4. In preferred embodiments L1 has a length of 0, i.e. is absent or has a length of 10 amino acids, preferably between 1 to 5 amino acids. L3 serves a similar function as L1, i.e. it is too short to allow interaction between VC2 and VC3. Accordingly, in preferred embodiments L3 has a length of 0, i.e. is absent or has a length of 10 amino acids, preferably between 1 to 5 amino acids. In contrast L2 must be sufficiently long to allow interaction between VC1 and VC2 as well as VC3 and VC4. This is best achieved with linker of more than 10 amino acids length, preferably between 12 to 20 amino acids, more preferably 14 to 18 amino acids.

In the trivalent binding molecule of the present invention, the second polypeptide may have the structure of: VC5-L4-VC6. As outlined above a linker L4 with a length of 10 or less amino acids will prevent the interaction of VC5 and VC6 and, thus prevent the formation of a binding site. Accordingly, linker L4 preferably has a length of more than 10 amino acids, preferably between 12 to 20 amino acids, more preferably 14 to 18 amino acids.

According to one embodiment, the linkers can be the same or different. According to a preferred embodiment, the linkers are selected from the group consisting of but not limited to SEQ ID NO:1, SEQ ID NO:2, SEQ ID NO:3, and SEQ ID NO:4. Most preferably linker 1 (L1) is a peptide having the sequence set forth in SEQ ID NO:1, linker 2 (L2) is a peptide having the sequence set forth in SEQ ID NO:2, linker 3 (L3) is a peptide having the sequence set forth in SEQ ID NO:1, linker 4 (L4) is a peptide having the sequence set forth in SEQ ID NO:2, linker 5 (L5) is a peptide having the sequence set forth in SEQ ID NO:3, linker 6 (L6) is a peptide having the sequence set forth in SEQ ID NO:4, and linker 7 (L7) is a peptide having the sequence set forth in SEQ ID NO:4. The linkers in the first polypeptide can therefore be selected in a way that favors correct assembly of the scDVAP and the third binding site and avoiding incorrect assembly into non-functional binding sites.

According to the present invention, two of the binding sites of the trivalent binding molecule specifically bind to the same or different antigens. Antigens in this respect explicitly do not include trigger molecules on an immune effector cell. The trivalent binding molecule of the present invention preferably binds to antigens that are overexpressed on tumor cells and include receptor-tyrosine-kinases, such as EGFR, HER2, HER3, HER4, ROR1, ROR2, cMET, AXL, RET, ALK, FGFR2 and IGF-1R, cell adhesion molecules such as CEA, EpCAM, members of the TNF receptor-superfamily, such as DR4, DR5, Fas, TNFR1 and TNFR2, or are overexpressed on cells of the tumor-microenvironment, such as FAP and CD105, but not limited thereto. A preferred antigen is HER3 or EGFR. According to a preferred embodiment, the trigger molecule is CD3 and the antigen is HER3.

Although it is not required that the first and the second binding sites of the first polypeptide bind to an antigen and a trigger molecule on an immune effector cell, respectively, and the third binding site of the second polypeptide binds to the same or a different antigen, the preferred embodiment of the present invention is directed to a trivalent binding molecule in which the first polypeptide comprises binding domains against an immune effector cell and an antigen, and the second polypeptide comprises a binding site against an antigen. More preferably, the second polypeptide comprises a binding site that is directed against the same antigen as the antigen binding site of the first polypeptide.

According to a preferred embodiment, the two binding sites specifically binding to antigens bind the same antigen. The trivalent binding molecule of the present invention according to this embodiment is thus bispecific with two binding sites directed to the same antigen and one binding site directed against a trigger molecule on an immune effector cell.

According to the present invention, the one or more antigens to which the binding molecule of the present invention may bind can be selected form the group consisting of but not limited to: ABCF1; ACVR1; ACVR1B; ACVR2; ACVR2B; ACVRL1; ADORA2A; Aggrecan; AGR2; AMHR2; AR; AXL; AZ GP1(zinc-a-glycoprotein); B7.1; B7.2; BAFF-R; BCMA; BLR1 (MDR15); BlyS; BMPR1A; BMPR1B; BMPR2; B7-H3; C5R1; CASP1; CCR1 (CKR1/HM145); CCR2 (mcp-1RB/RA); CCR3 (CKR3 CMKBR3); CCR4; CCR5 (CMKBR5/ChemR13); CCR6 (CMKBR6/CKR-L3 STRL22/DRY6); CCR7 (CKR7 EB11); CCR8 (CMKBR81/TER1/CKR-L1); CCR9 (GPR-9-6); CD164; CD5; CD7; CD15; CD19; CD1G; CD11a; CD20; CD200; CD22; CD23; CD24; CD25; CD27; CD28; CD30; CD33; CD37; CD38; CD3E; CD3G; CD3Z; CD4; CD40; CD40L; CD41; CD4SRB; CD51; CD52; CD56; CD6; CD70; CD72; CD73; CD74; CD79A; CD79B; CDB; CD80; CD81; CD83; CD86; CD105; CD117; CD123; CD125; CD137L; CD137; CD147; CD152; CD154; CD221; CD276; CD279; CD319; CDH1 (Ecadherin); CDH10; CDH12; CDH13; CDH18; CDH19, CDH20; CDH5; CDH7; CDH8; CDH9; CEA; CEACAM5; CKLFSF2; CKLFSF3; CKLFSF4; CKLFSF5; CKLFSF6; CKLFSF7; CKLFSF8; CLDN3; CLDN6 (claudin-6); CLDN7 (claudin-7); CLDN18.2; CLN3; cMET; CMKLR1; CMKOR1 (RDC1); CNR1; CR2; CSFR1; CTLA-4; CXCR3 (GPR9/CKR-L2); CXCR4; CXCR6 (TYMSTR/STRL33/Bonzo); CYSLTR1; DLL3; DPP4; DR3; DR4; DR5; DR6; EDAR; EDA2R; EDG1; EpCAM; EGFR; ENG; EPHA3; EPHB4; ESR1; ESR2; FAP; FCER1A; FCER2; FCGR3A; FGFR1; FGFR2; FGFR3; FGFR4; FLT1; folate receptor 1; FY (DARC); GABRP (GABAa); GD2; GD3; GITR; GPNMB; GPR2 (CCR10); GPR31; GPR44; GPR81 (FKSG80); GRP; HAVCR2; HER2; HER3; HER4; histamine and histamine receptors; HLA-A; HLA-DRA; HM74; HMW-MAA HVEM; TNF-RHUMCYT2A; ICAM-1; IGF1R; IGP1R; IGHE; IL10RA; IL10RB; IL11RA; IL12RB1; IL12RB2; IL13RA1; IL13RA2; IL15RA; IL17RIL18BP; IL18R1; IL18RAP; IL1R1; IL1R2; ILLRAP; IL1RAPL1; IL1RAPL2; IL1RL1; IL1RL2; IL1RN; IL20RA; IL21R; IL22R; IL22RA2L29; IL2RA; IL2RB; IL2RG; IL3RA; IL4R; IL5RA; IL6R; IL6ST (glycoprotein 130); IL7R; IL8RA; IL8RB; IL8RB; IL9R; integrin α_{ν}β₃; integrin β₇; ITGA2; ITGA3; ITGA6 (a6 integrin); ITGAV; ITGB3; ITGB4 (b4 integrin); KDR; KIR2D; LEY; Lingo-p75; Lingo-Troy; LTB4R (GPR16); LTB4R2; LTBR; MCAM; MCSP; MET; MER; MSLN; MS4A1; MT3 (metalothionectin-III); MTSS1; MUC1(mucin); MUC2; MUC16; NGFR; NgRLingo; NOGO-A; NgR-Nogo66 (Nogo); NgR-p75; NgR-Troy; OPRD1; OX40; P2RX7; PAWR; PCDC1; PCNA; PCSK9; PD1; PDGR; igfPECAM1; uPAR; PR1; PSCA; PSMA; PTAFR; VEGFR1; VEGFR2; VEGFR3; RANK; RARB; RELT; RET; ROBO2; RON; ROR1; ROR2; RYK; S100A2; TAG-72; tau protein; TB4R2; TEK; TGFBR1; TGFBR2; TGFBR3; TIE (Tie-1); TIE-1; TIE-2; TIMP3; tissue factor; TLR10; TLR2; TLR3; TLR4; TLRS; TLR6; TLR7; TLR8; TLR9; TNFRSF11A; TNFRSF1A; TNFRSF1B; TNFRSP21; TNFRSF5; TNFRSF6 (Fas); TNFRSF7; TNFRSF8; TNFRSF9; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TNF-R1; TNF-R2; Toll-like receptors; TRAIL-R1; TRAIL-R2; TRAIL-R3; TRAIL-R4; TREM1; TREM2; TRPC6; TROY; TWEAK; TYRO3; TYRP1; VAP-1; versican; VLA-4.

According to the present invention, only one of the three binding sites of the trivalent binding molecule of the present invention is directed against and thus binds a trigger molecule on an immune effector cell. According to the present invention, such trigger molecules are expressed by cells of the immune system to regulate their activity. Non-limiting examples of such trigger molecules are CD2, CD3, CD16, CD44, CD64, CD69, CD89, Mel14, Ly-6.2C, TCR-complex, Vy9V52 TCR, and NKG2D.

In a particular embodiment according to the first aspect of the invention, it is preferred that the first binding site of the scDVAP and the third binding site of the second polypeptide specifically bind the same or a different antigen, and the second binding site of the scDVAP specifically binds a trigger molecule on an immune effector cell. Alternatively, the first binding site of the scDVAP and the second binding site of the scDVAP specifically bind the same or a different antigen, and the third binding site of the second binding module specifically binds a trigger molecule on an immune effector cell.

According to an embodiment of the present invention, the scDVAP is a single chain diabody, i.e. bivalent and bispecific antibody fragments connected by a peptide linker to form one single polypeptide chain.

According to a preferred embodiment, the second polypeptide is selected from the group consisting of a single variable heavy or light chain domain, an scFv, and a Fab.

According to the present invention, the first and the second polypeptide are interconnected to form the trivalent binding molecule. This connection can be achieved by means of a fifth peptide linker (L5), a peptide bond, a chemical bond or by one or more dimerization domains. Particularly preferred is one or more dimerization domains. Preferably, the one or more dimerization domain is selected from the group consisting of an Fc region, a heterodimerizing Fc region, CH1/CL, EHD2, MHD2, hetEHD2, the last heavy chain domain (CH3 or CH4) of IgG, IgD, IgA, IgM, or IgE and heterodimerizing derivatives thereof, as well as constant domains of T Cell Receptors (TCR) such as the constant regions of TCR α-, β-, γ-, and δ-chains. Particularly preferred is an Fc region or a heterodimerizing Fc region. Such Fc region or heterodimerizing Fc region (denoted as Fc part in the following) has the advantage of providing an increased flexibility to the molecule, which allows using different linkers with different sizes and compositions, thereby increasing flexibility. Further, an Fc part may change the structure of the molecule. By introducing the Fc part, different structures of the molecule can be created, e.g. by adding the second part (e.g. the scFv molecule) of the molecule to the C-terminus of the Fc part. In addition, an Fc part allows conjugation with further components of the molecule, e.g. modification with drugs or with additional binders or functional domains on the C-terminus of the molecule. Therefore, according to a preferred embodiment of the invention, in the trivalent binding molecule of the invention, the first and second polypeptides are interconnected by one or more dimerization domains. The Fc region is preferably a silenced Fc region, which is also referred to as effector-deficient Fc region. As used herein, an “effector-deficient” Fc region is defined as an Fc region that has been altered so as to reduce or eliminate Fc-binding to CD16, CD32, and/or CD64 type IgG receptors. The reduction in Fc-binding for a silenced Fc region to CD16, CD32, and/or CD64 preferably is essentially a complete reduction as compared to an effector-competent control. An essentially complete reduction may also be present if the reduction is for about 80%, about 90%, or about 95%, or more, as compared to an effector-competent control. Methods for determining whether a binding molecule has a reduced Fc-binding to CD16, CD32, and/or CD64 are well known in the art and are described e.g. in US20110212087 A1 and WO 2013165690.

According to a preferred embodiment, the first binding module is connected to a first heterodimerizing domain and the second binding module is connected to a second heterodimerizing domain. The connection is preferably via a peptide bond or a linker, wherein the linker can be as described and defined above. The heterodimerizing domains of the first and second polypeptide preferably bind to each other through hydrophobic and/or electrostatic interactions. Examples of such heterodimerizing domains include heterodimerizing Fc parts such as those defined above.

According to the present invention, the immune effector cell is preferably selected from the group consisting of T-cells, natural killer cells, natural killer T cells, macrophages, and granulocytes. Particularly preferred immune effector cells are T cells.

According to a preferred embodiment, the trivalent binding molecule of the present invention further comprises one or more of a peptide leader sequence, one or more molecules that aid in purification, one or more co-stimulatory molecules, and/or checkpoint inhibitors. Molecules that aid in purification are preferably one or more hexahistidyl-tags and/or one or more FLAG-tags. Co-stimulatory molecules include but are not limited to B7.1, B7.2, 4-1BBL, LIGHT, ICOSL, GITRL, CD27L, CD40L, OX40L, and CD70, and derivatives or combinations thereof. Checkpoint inhibitors are for example PD-L1 and PD-L2. An exemplary peptide leader sequence is an Igκ chain leader sequence.

Single-chain diabodies (scDb) are derivatives of diabodies in which the four variable domains of two antibodies are connected by 3 linkers. In contrast to diabodies, DART molecules or disulfide-stabilized diabodies, the scDb composition requires expression of only a single polypeptide chain. The scDb format facilitates correct assembly of the variable domains into functional molecules (Völkel et al., 2001, Protein Eng. 14:815-823). It was shown that scDb can be employed for T-cell retargeting to tumor cells but also cells of the tumor microenvironment (Müller et al., 2007, J. Biol. Chem. 282: 12650-12660; Korn et al., 2004, J. Immunother. 27:99-106; Müller et al., 2008, J. Immunother. 31:714-722). Compared to tandem scFv, the format used to generate so-called BiTE such as Blinatumomab, single-chain diabodies adopt a rather rigid structure and exhibit an improved stability (Korn et al., 2004, J. Gene Med. 6:642-651). Furthermore, they allow a very close linkage of effector and target cells due to the small distance between the two binding sites, which is only approximately 5 nm.

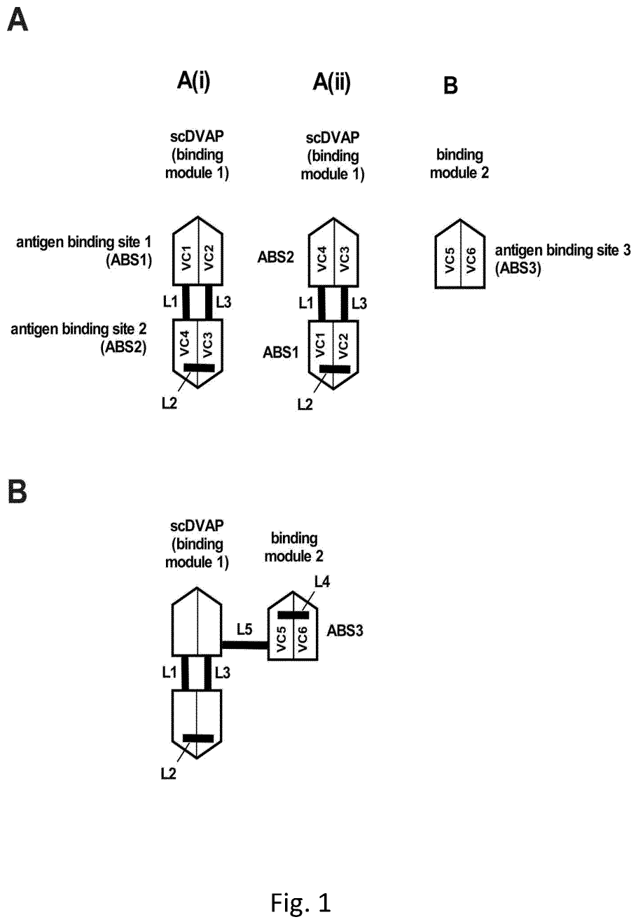

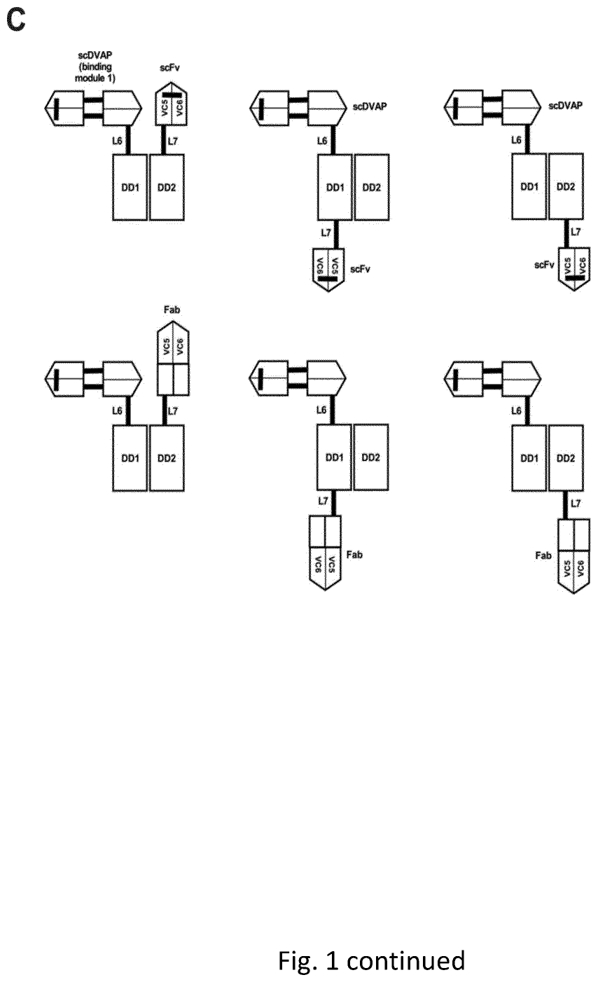

According to the present invention, the scDb format was used to generate novel trivalent bispecific molecules by combining a bivalent scDb with an additional binding site, e.g. a scFv or a Fab fragment. This was either achieved by directly fusing a scDb with a scFv (scDb-scFv) (FIGS. 1A and B) or by fusing the scDb moiety to a first heterodimerizing Fc (hetFc1) chain and either a scFv or a Fab to a second heterodimerizing Fc chain (hetFc2) (FIG. 1 C). To generate these types of molecules, only one polypeptide chain (scDb-scFv), two polypeptide chains (scDb/scFv-Fc) or three polypeptide chains (scDb/Fab-Fc) are required for producing these molecules.

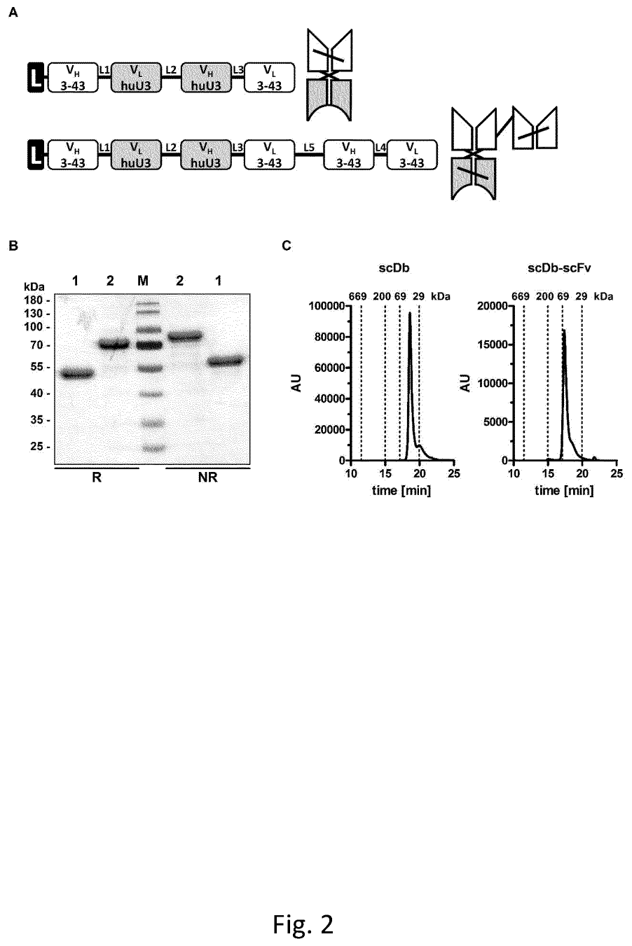

According to a preferred embodiment, the trivalent binding molecule of the present invention may thus have the form of:
(i) VC1-L1-VC4-L2-VC3-L3-VC2-L5*-VC5-L4-VC6; or
(ii) VC4-L1-VC1-L2-VC2-L3-VC3-L5*-VC5-L4-VC6,

wherein L5* is a peptide linker, a peptide bond, or a chemical bond, preferably a peptide linker as defined above. The combination of the scDVAP with a scFv in a single peptide chain is also denoted as scDb-scFv. FIGS. 1B and 2A show a respective scDb-scFv, wherein the one shown in FIG. 2A is based on HER3 (3-43) and huU3 heavy and light chains.

The linkers L1, L2, L3, L4, L5, L5*, L6, and L7 can be the same or different. Preferably, L1, L2, L3, L4, L5, L5*, L6, and L7 are as defined above. Preferred peptide linkers are GGGGS (SEQ ID NO:1), [G₄S]₃ (SEQ ID NO:2), AAAGGSGGGGSGGGT (SEQ ID NO:3), and AAA (SEQ ID NO:4). According to a most preferred embodiment, most preferably linker 1 (L1) is a peptide having the sequence set forth in SEQ ID NO:1, linker 2 (L2) is a peptide having the sequence set forth in SEQ ID NO:2, linker 3 (L3) is a peptide having the sequence set forth in SEQ ID NO:1, linker 4 (L4) is a peptide having the sequence set forth in SEQ ID NO:2, linker 5* (L5*) is a peptide having the sequence set forth in SEQ ID NO:3.

Thus, according to a preferred embodiment, the trivalent binding molecule of the present invention is a scDb-scFv, which preferably further comprises a peptide leader sequence such as an Igκ chain leader sequence on the N-terminus of VC1 or VC4, and/or a hexahistidyl-tag or FLAG-tag on the C-terminus of VC6.

According to a further preferred embodiment, the trivalent binding molecule of the present invention may thus have the form of:
(i) VC1-L1-VC4-L2-VC3-L3-VC2-L6-DD1=DD2-L7-VC5-L4-VC6; or
(ii) VC4-L1-VC1-L2-VC2-L3-VC3-L6-DD1=DD2-L7-VC5-L4-VC6, as is also exemplary shown for specific embodiments in FIG. 1C (scDb/scFv-Fc).

Linkers L1 to L4 and L6 to L7 are as defined above. Most preferably linker 1 (L1) is a peptide having the sequence set forth in SEQ ID NO:1, linker 2 (L2) is a peptide having the sequence set forth in SEQ ID NO:2, linker 3 (L3) is a peptide having the sequence set forth in SEQ ID NO:1, linker 4 (L4) is a peptide having the sequence set forth in SEQ ID NO:2, linker 6 (L6) is a peptide having the sequence set forth in SEQ ID NO:4, and linker 7 (L7) is a peptide having the sequence set forth in SEQ ID NO:4.

In an alternative embodiment, the second polypeptide is a Fab fragment comprising VC5 and VC6. In these cases, the Fab fragment may have the structure of for example VC5-CH1 and VC6-CL or vice versa. Accordingly, the trivalent binding molecule of the present invention according to a further preferred embodiment may have the form of:
(i) VC1-L1-VC4-L2-VC3-L3-VC2-L6-DD1=DD2-L7-Fab; or
(ii) VC4-L1-VC1-L2-VC2-L3-VC3-L6-DD1=DD2-L7-Fab,
   as is also exemplary shown for specific embodiments in FIG. 1C (scDb/Fab-Fc). In the above shown structures, DD1 and DD2 are representatives of dimerizing domains as defined above, such as hetFc1 and hetFc2. Linkers L1 to L3 are as defined above. Most preferably linker 1 (L1) is a peptide having the sequence set forth in SEQ ID NO:1, linker 2 (L2) is a peptide having the sequence set forth in SEQ ID NO:2, linker 3 (L3) is a peptide having the sequence set forth in SEQ ID NO:1, linker 6 (L6) is a peptide having the sequence set forth in SEQ ID NO:4, and linker 7 (L7) is a peptide having the sequence set forth in SEQ ID NO:4.

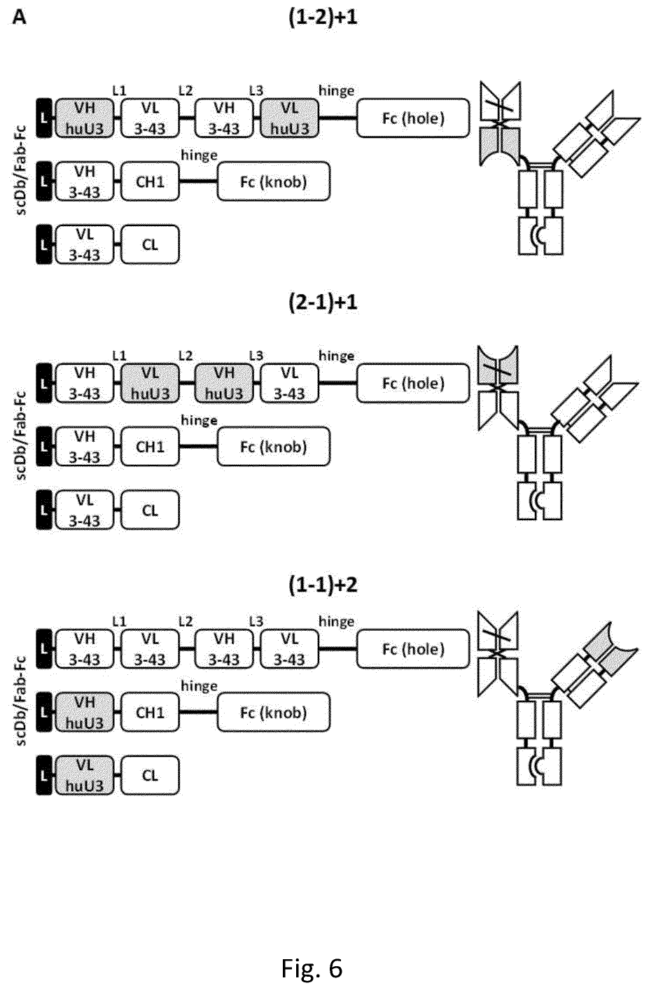

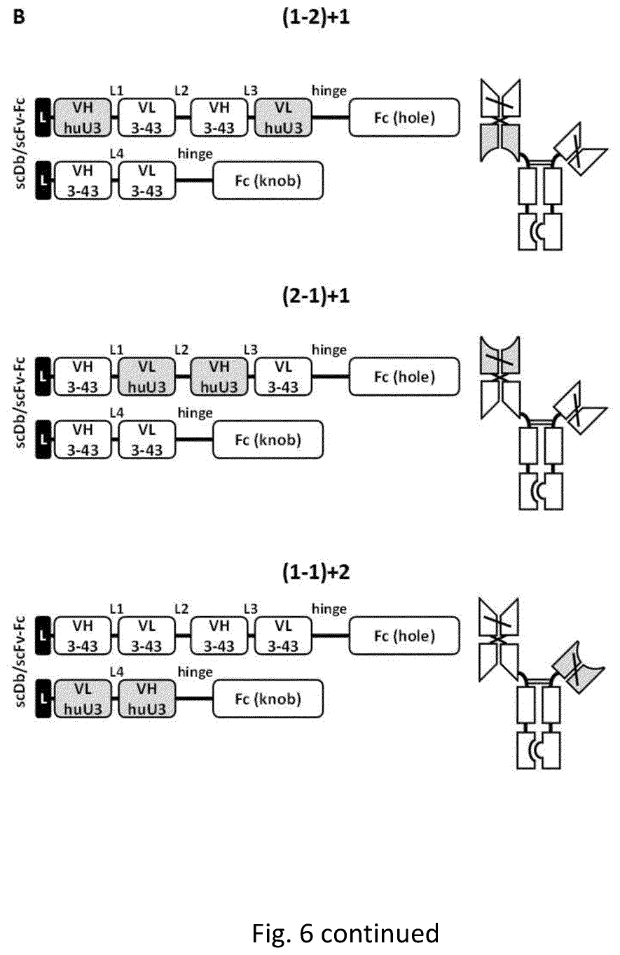

Preferred embodiments of the trivalent binding molecule according to the present invention are shown in FIG. 1C and FIG. 6.

The present inventors surprisingly found that the trivalent binding molecules of the present invention exhibit a strongly increased target cell binding and increased cytotoxic potential compared to the bivalent bispecific molecules having only one binding site for the tumor target antigen.

Particularly preferred binding molecules according to the invention are binding molecules targeting HER3. Particularly preferred are binding molecules comprising (1) SEQ ID NO: 7 and 9; (2) SEQ ID NO: 7, 10 and 11; (3) SEQ ID NO: 8 and 9; or (4) SEQ ID NO: 8, 10 and 11. Further preferred molecules according to the invention comprise the combination of sequences identified above as (1) to (4) having at least 90% sequence identity to the sequences identified above under (1) to (4), preferably 91%, 92%, 93%, 94%, 95%, 96%, 97%, 98% or 99% sequence identity to the sequences identified above under (1) to (4). Preferably, these molecules with a sequence identity of at least 90% or more to the sequences identified above under (1) to (4) further maintain essentially the same or maintain the same biological function as the respective molecule from which it is derived comprising the sequences identified above under (1) to (4). Preferably, the term “biological function” as used herein refers to binding specificity and/or affinity. Maintaining essentially the same biological function means a binding specificity and/or affinity of at least 50% of that of the respective molecule comprising the sequences identified above under (1) to (4) from which it is derived, preferably a binding specificity and/or affinity of at least 60%, 70%, 80%, 85%, 90%, 91%, 92%, 93%, 94%, 95%, 96%, 97%, 98%, 99%. Determining binding and/or affinity is well known to the skilled person and can be performed e.g. by surface plasmon resonance measurements and/or by in vitro release assays.

It will be understood by the skilled person that the molecules according to the invention may or may not carry a histidine-tag (His-tag, 6×His tag etc.) or a similar addition or tag as exemplarily described herein at one or more of the polypeptides in order to facilitate easy purification thereof. The skilled person readily understands that such additional structure does not have a specific influence on the molecule's functional characteristics as described herein.

According to a further aspect, the present invention provides a nucleic acid or set of nucleic acids encoding the trivalent binding molecule of the present invention.

According to a further aspect, the present invention also provides a vector comprising the nucleic acid or set of nucleic acids of the present invention.

According to a further aspect, the present invention also provides a pharmaceutical composition comprising the trivalent binding molecule, the nucleic acid or set of nucleic acids, or the vector of the present invention, and a pharmaceutically acceptable carrier.

According to a further aspect, the present invention also provides the trivalent binding molecule, the nucleic acid or set of nucleic acids, the vector, or the pharmaceutical composition of the present invention for use in medicine. In particular, the present invention provides the trivalent binding molecule, the nucleic acid or set of nucleic acids, the vector, or the pharmaceutical composition of the present invention for use in treating cancer, a viral infection or an autoimmune disease. Preferably, the cancer is selected from the group consisting of carcinoma, sarcoma, lymphoma, leukemia, germ cell tumor and blastoma.

According to a further aspect, the present invention provides a method of treating cancer, a viral infection or an autoimmune disease in a patient in need thereof, comprising administering to the patient the trivalent binding molecule, the nucleic acid or set of nucleic acids, the vector, or the pharmaceutical composition of the present invention. According to a preferred embodiment, the cancer is selected from the group consisting of carcinoma, sarcoma, lymphoma, leukemia, germ cell tumor and blastoma.

According to a further aspect, the present invention provides a method of inhibiting metastatic spread of a cell, comprising contacting the cell with the trivalent binding molecule, the nucleic acid or set of nucleic acids, the vector, or the pharmaceutical composition of the present invention.

In particular, the present invention pertains to the following items:

Item 1. A trivalent binding molecule comprising:
(A) a first polypeptide comprising a single-chain dual valence antigen binding polypeptide (scDVAP), wherein the scDVAP comprises a first binding domain comprising a first variable chain (VC1) and a second variable chain (VC2), and a second binding domain comprising a third variable chain (VC3) and a fourth variable chain (VC4), wherein VC1 and VC2 together form a first antigen binding site, and VC3 and VC4 together form a second antigen binding site, wherein
(i) VC1 and VC4 are connected by a first peptide linker (L1), VC4 and VC3 are connected by a second peptide linker (L2), and VC3 and VC2 are connected by a third peptide linker (L3), or
(ii) wherein VC4 and VC1 are connected by a first peptide linker (L1), VC1 and VC2 are connected by a second peptide linker (L2), and VC2 and VC3 are connected by a third peptide linker (L3),
(B) a second polypeptide comprising a third binding domain comprising a fifth variable chain (VC5) and a sixth variable chain (VC6), wherein VC5 and VC6 together form a third antigen binding site,
wherein
(a) two of the binding sites of the trivalent binding molecule specifically bind to the same or different antigens which is not a trigger molecule on an immune effector cell,
(b) only one of the binding sites of the trivalent binding molecule is directed against a trigger molecule on an immune effector cell, and
(c) the first and second polypeptide are interconnected.

Item 2. The trivalent binding molecule according to item 1, wherein:
(i) the first binding site of the scDVAP and the third binding site of the second polypeptide specifically bind the same or a different antigen, and the second binding site of the scDVAP specifically binds a trigger molecule on an immune effector cell; or
(ii) the first binding site of the scDVAP and the second binding site of the scDVAP specifically bind the same or a different antigen, and the third binding site of the second binding module specifically binds a trigger molecule on an immune effector cell.

Item 3. The trivalent binding molecule according to item 1 or 2, wherein the variable chains (VCs) are each selected from the group consisting of a TCR α-chain variable domain, TCR β-chain variable domain, variable light (V_{L}) chain domain and variable heavy (V_{H}) chain domain.

Item 4. The trivalent binding molecule according to any one of items 1 to 3, wherein the scDVAP is a single chain diabody.

Item 5. The trivalent binding molecule according to any of items 1 to 4, wherein VC5 and VC6 are connected by a fourth peptide linker (L4)

Item 6. The trivalent binding molecule according to any of items 1 to 5, wherein the two binding sites specifically binding to antigens bind the same antigen.

Item 7. The trivalent binding molecule according to any of items 1 to 4, wherein the second polypeptide is selected from the group consisting of a single variable heavy or light chain domain, an scFv, and a Fab fragment.

Item 8. The trivalent binding molecule according to any of items 1 to 7, wherein the first and second polypeptides are interconnected by a fifth peptide linker (L5) or a dimerization domain, a peptide bond, a disulfide bond or by one or more dimerization domains.

Item 9. The trivalent binding molecule according to item 8, wherein the one or more dimerization domain is selected from the group consisting of an Fc region, a heterodimerizing Fc region, CH1/CL, EHD2, MHD2, hetEHD2, the last heavy chain domain (CH3 or CH4) of IgG, IgD, IgA, IgM, or IgE and heterodimerizing derivatives thereof, and the constant C-alpha and C-beta domains of a T cell receptor (TCR).

Item 10. The trivalent binding molecule according to item 9, wherein the first binding module is connected, preferably via a peptide bond or a linker (L6), to a first heterodimerizing domain, and the second binding module is connected, preferably via a peptide bond or a linker (L7), to the same or a second heterodimerizing domain.

Item 11. The trivalent binding molecule according to item 10, wherein the heterodimerizing domains of the first and second polypeptide bind to each other through hydrophobic and/or electrostatic interactions.

Item 12. The trivalent binding molecule according to any of items 1 to 11, wherein the immune effector cell is selected from the group consisting of T-cells, natural killer cells, natural killer T cells, macrophages, and granulocytes.

Item 13. The trivalent binding molecule according to any of items 1 to 12, wherein the trigger molecule of the immune effector cell is selected from the group consisting of CD2, CD3, CD16, CD44, CD64, CD69, CD89, Mel14, or Ly-6.2C.

Item 14. The trivalent binding molecule according to any of items 1 to 13, wherein the antigen is a tumor-associated antigen, preferably wherein the tumor-associated antigen is selected from the group consisting of EGFR, EGFRvIII, HER2, HER3, HER4, cMET, RON, FGFR2, FGFR3, IGF-1R, AXL, Tyro-3 MerTK, ALK, ROS-1, ROR-1, ROR-2, RET, MCSP, FAP, Endoglin, EpCAM, claudin-6, claudin 18.2, CD19, CD20, CD22, CD30, CD33, CD52, CD38, CD123, BCMA, CEA, PSMA, DLL3, FLT3, gpA33, SLAM-7, CCR9.

Item 15. The trivalent binding molecule according to items 1 to 14, further comprising one or more of:
(a) a peptide leader sequence;
(b) one or more molecules that aid in purification, preferably a hexahistidyl-tag or FLAG-tag;
(c) one or more co-stimulatory molecules and/or checkpoint inhibitors.

Item 16. A nucleic acid or set of nucleic acids encoding the trivalent binding molecule according to any of items 1 to 15.

Item 17. A vector comprising the nucleic acid or set of nucleic acids of item 16.

Item 18. A pharmaceutical composition comprising the trivalent binding molecule according to any of items 1 to 15, the nucleic acid or set of nucleic acids of item 16 or the vector of item 17, and a pharmaceutically acceptable carrier.

Item 19. The trivalent binding molecule according to any of items 1 to 15, the nucleic acid or set of nucleic acids of item 16, the vector of item 17 or the pharmaceutical composition according to item 18, for use in medicine.

Item 20. The trivalent binding molecule according to any of items 1 to 15, the nucleic acid or set of nucleic acids of item 16, the vector of item 17 or the pharmaceutical composition according to item 18, for use in treating cancer, a viral infection or an autoimmune disease.

Item 21. The trivalent binding molecule, the nucleic acid, the vector or the pharmaceutical composition for use according to item 20, wherein the cancer is selected from the group consisting of carcinoma, sarcoma, lymphoma, leukemia, germ cell tumor and blastoma.

Item 22. A method of treating cancer, a viral infection or an autoimmune disease in a patient in need thereof, comprising administering to the patient the trivalent binding molecule according to any of items 1 to 15, the nucleic acid or set of nucleic acids of item 16, the vector of item 17 or the pharmaceutical composition according to item 16.

Item 23. The method of item 22, wherein the cancer is selected from the group consisting of carcinoma, sarcoma, lymphoma, leukemia, germ cell tumor and blastoma.

Item 24. A method of inhibiting metastatic spread of a cell, comprising contacting the cell with the trivalent binding molecule according to any of items 1 to 15, the nucleic acid or set of nucleic acids of item 16, the vector of item 17 or the pharmaceutical composition according to item 18.

Item 25. The trivalent binding molecule according to any one of items 1 to 15, comprising an effector-deficient Fc region.

Item 26. A trivalent binding molecule comprising SEQ ID NO: 7 and 9.

Item 27. A trivalent binding molecule comprising SEQ ID NO: 7, 10 and 11.

Item 28. A trivalent binding molecule comprising SEQ ID NO: 8 and 9.

Item 29. A trivalent binding molecule comprising SEQ ID NO: 8, 10 and 11.

Item 30. A nucleic acid or set of nucleic acids encoding the binding molecule according to any of items 25 to 29.

Item 31. A vector comprising the nucleic acid or set of nucleic acids of item 30. Item 32. A host cell comprising the vector according to item 31.

Item 33. A pharmaceutical composition comprising the binding molecule according to any of items 25 to 29, the nucleic acid or set of nucleic acids according to item 30, the vector according to item 31, or the host cell according to item 32, and a pharmaceutically acceptable carrier.

Item 34. The binding molecule according to any of items 25 to 29, the nucleic acid or set of nucleic acids according to item 30, the vector according to item 31, the host cell according to item 32, or the pharmaceutical composition according to item 33 for use in medicine, preferably for use in the treatment of cancer.

Item 35. A method of treatment, comprising administering to a patient in need thereof a therapeutically effective amount of the binding molecule according to any of items 25 to 29, the nucleic acid or set of nucleic acids according to item 30, the vector according to item 31, the host cell according to item 32, or the pharmaceutical composition according to item 33.

Item 36. A method of treating cancer, comprising administering to a patient in need thereof a therapeutically effective amount of the binding molecule according to any of items 25 to 29, the nucleic acid or set of nucleic acids according to item 30, the vector according to item 31, the host cell according to item 32, or the pharmaceutical composition according to item 33.

Item 37. The binding molecule according to any of items 1 to 29, the nucleic acid or set of nucleic acids according to item 30, the vector according to item 31, the host cell according to item 32, or the pharmaceutical composition according to item 33 for use in medicine, preferably for use in the treatment of cancer.

Item 38. A method of treatment, comprising administering to a patient in need thereof a therapeutically effective amount of the binding molecule according to any of items 1 to 29, the nucleic acid or set of nucleic acids according to item 30, the vector according to item 31, the host cell according to item 32, or the pharmaceutical composition according to item 33.

Item 39. A method of treating cancer, comprising administering to a patient in need thereof a therapeutically effective amount of the binding molecule according to any of items 1 to 29, the nucleic acid or set of nucleic acids according to item 30, the vector according to item 31, the host cell according to item 32, or the pharmaceutical composition according to item 33.

### EXAMPLES

### Example 1: Production of a Trivalent, Bispecific scDb-scFv Fusion Protein Targeting HER3 and CD3

A trivalent anti-HER3×anti-CD3 bsAb (hereafter referred to as scDb-scFv) was generated by fusing an anti-HER3 scFv to the C-terminus of an anti-HER3×CD3 bispecific scDb (FIG. 2A). The anti-HER3 binding site was derived from antibody IgG 3-43 directed against domain III and part of domain IV of HER3 (Schmitt et al., 2017, mAbs, 9:831-843). The CD3 binding site consists of a humanized version of the anti-CD3 mAb UCHT1. Both, the scDb (SEQ ID NO: 5) and the scDb-scFv (SEQ ID NO: 6) were produced in transiently transfected HEK293-6E cells (NRC Biotechnology Research Institute, Canada) using polyethylenimine (PEI; linear, 25 kDa, Sigma-Aldrich, 764604). The plasmids for transfection are based on the pSecTagAL1 vector (a modified version of pSecTagA (Invitrogen, Thermo Fisher Scientific, V90020)). Supernatants were harvested 96 hours post transfection, proteins were precipitated by addition of 390 g/L (NH₄)₂SO₄, purified by immobilized metal ion affinity chromatography (IMAC) followed by size-exclusion FPLC on a Superdex 200 10/300 GL column (PBS as mobile phase, 0.5 ml/min flow rate). Production yields ranged from 7 mg/l (scDb) to 0.4 mg/l (scDb-scFv). Protein purity was confirmed by SDS-PAGE analysis, where both proteins migrated according to their calculated molecular mass (scDb: 55.4 kDa; scDb-scFv: 82.5 kDa) (FIG. 2B). Integrity of the proteins was determined using Waters 2695 HPLC and a TSKgel SuperSW mAb HR column (Tosoh Bioscience) at a flow rate of 0.5 ml/min with 0.1 M Na₂HPO₄/NaH₂PO₄, 0.1 M Na₂SO₄, pH 6.7 as mobile phase. Thyroglobulin (669 kDa, Sr 8.5 nm), β-Amylase (200 kDa, Sr 5.4 nm), bovine serum albumin (67 kDa, Sr 3.55 nm) and carbonic anhydrase (29 kDa, Sr 2.35 nm) were used as reference proteins. In size-exclusion chromatography, all proteins eluted as one major peak (FIG. 2C).

### Example 2: Cell Binding of a Trivalent, Bispecific scDb-scFv Fusion Protein Targeting HER3 and CD3

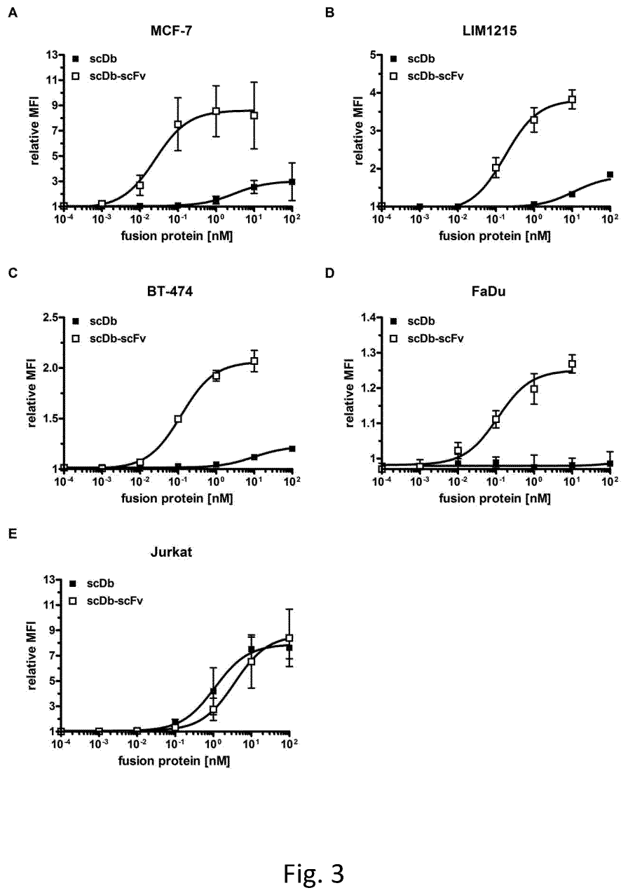

Cell binding studies were performed by flow cytometry using cell lines with different expression levels of HER3 (Table 1). Adherent cells were washed with PBS and shortly trypsinized at 37° C. Trypsin was quenched with FCS-containing medium and removed by centrifugation (500×g, 5 min.). 1×10⁵ target cells (MCF-7, Jurkat, BT-474, FaDu, or LIM1215) were incubated with a serial dilution of recombinant proteins for 1 hour at 4° C. Bound protein was detected using a PE-conjugated anti-hexahistidyl tag mAb (Miltenyi Biotec). Incubation and washing steps were performed in PBS, 2% FBS, and 0.02% sodium azide. Fluorescence was measured by MACSQuant VYB (Miltenyi Biotec) and data were analyzed using FlowJo (Tree Star). Relative median fluorescence intensities (MFI) were calculated as followed: relative MFI=((MFIₛₐₘₚₗₑ−(MFI_{detection}−MFI_{cells}))/MFI_{cells}). On all HER3 expressing cell lines, scDb-scFv (SEQ ID NO: 6) showed superior binding properties compared to the scDb (SEQ ID NO: 5). EC₅₀ values for the scDb-scFv were in the low nanomolar range, whereas the scDb showed up to factor 50 weaker binding (FIG. 3A-D) (Table 1). Both, the scDb and the scDb-scFv showed similar binding to Jurkat cells with EC₅₀ values of 1.6±1.3 and 4.0±1.3 nM, respectively (FIG. 3E).

TABLE 1
Overview of target cell binding by scDb-scFv and scDb molecules.

| | | | EC₅₀ [pM] | |
|---|---|---|---|---|
| cell line | HER3 expression | | scDb-scFv | scDb |
| MCF-7 | 17,283 | HER3/cell | 30 ± 20 | 4,700 ± 6,200 |
| LIM1215 | 19,877 | HER3/cell | 200 ± 100 | 11,700 ± 6,000 |
| BT-474 | 11,244 | HER3/cell | 200 ± 170 | 9,700 ± 2,200 |
| FaDu | 2,884 | HER3/cell | 100 ± 30 | n.d. |

| | | | |
|---|---|---|---|
| Jurkat | — | 3,998 ± 1,270 | 1,638 ± 1,257 |

EC₅₀ values are shown in pM.
Mean ± SD, n.d. = not determined, n = 3.

### Example 3: T-Cell Activation a Trivalent, Bispecific scDb-scFv Fusion Protein Targeting HER3 and CD3

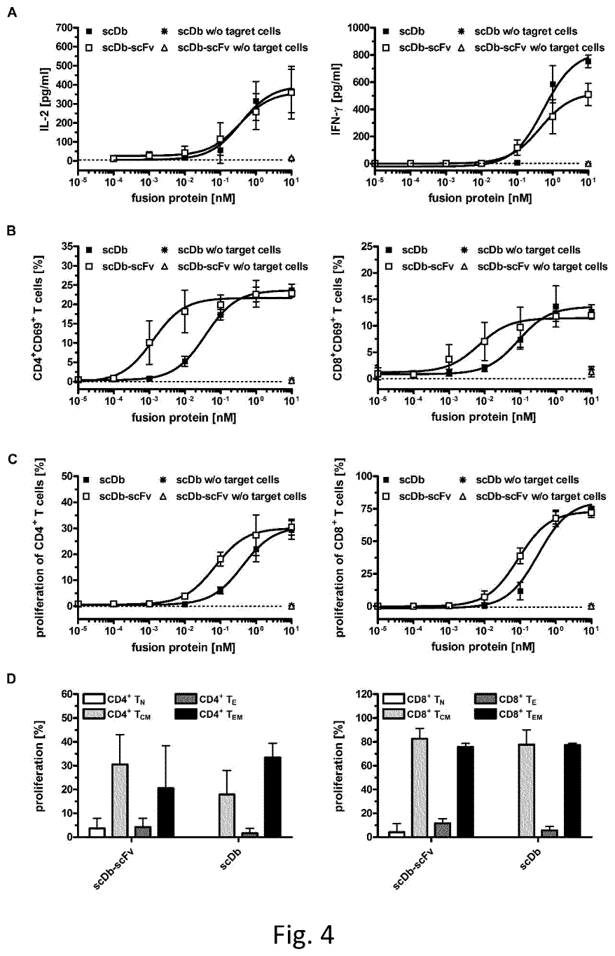

To address simultaneous binding of the scDb-scFv to tumor and effector cells, we investigated the activation of T-cells in a co-culture assay. First, we determined IL-2 and INF-γ release. 2×10⁴ MCF-7 cells/well were incubated with a serial dilution of scDb (SEQ ID NO: 5) and scDb-scFv (SEQ ID NO: 6) fusion proteins for 15 min at RT followed by addition of 2×10⁵ PBMCs/well. After 24 h (IL-2) or 48 h (INF-γ) of incubation at 37° C., cell-free supernatants of the co-cultures were harvested and concentrations of IL-2 and IFN-γ were determined using DuoSet sandwich ELISA kit (R&D Systems). Both, scDb-scFv and scDb showed a concentration-dependent cytokine release by T-cells. However, no significant differences in release of IL-2 or IFN-γ was observed. Additionally, neither scDb-scFv nor scDb was able to activate T-cells in terms of cytokine release in the absence of target cells (FIG. 4A) (Table 2).

Next, early activation of T-cells was determined by CD69-expression. 2×10⁴ MCF-7 cells/well were incubated with fusion proteins for 15 min followed by the addition of 2×10⁵ PBMCs/well. PBMCs were harvested after 24 h of incubation at 37° C. and CD69 expression on CD4⁺ and CD8⁺ T-cells was identified by flow cytometry using MACSQuant Analyzer 10 (Miltenyi Biotec). For both molecules, a dose-dependent activation of CD4⁺ and CD8⁺ T-cells was observed, whereby the scDb-scFv showed ˜30-fold and 8-fold lower EC₅₀ value in early activation of CD4⁺ and CD8⁺ T-cells, respectively, compared to the scDb (FIG. 4B) (Table 2).

Additionally, the effect of scDb-scFv and scDb on T-cell proliferation was investigated. Therefore, PBMCs were stained with carboxyfluorescein diacetate succinimidyl ester (CFSE, ThermoFisher) at 625 nM/1×10⁶ cells/ml following the manufacturer's instructions. 2×10⁴ MCF-7 cells/well were incubated with fusion protein for 15 min at RT followed by addition of 2×10⁵ CFSE-labeled PBMCs/well. After 6 days of incubation at 37° C., cells were harvested and immune cells of interest were labeled with fluorescence-conjugated antibodies directed against respective cell-surface markers (PerCP/Cy5.5 anti-human CD3 (Biolegend), PE anti-human CCR7 (Biolegend), APC anti-human CD45RA (Biolegend,), anti-human CD4-VioBlue (Miltenyi Biotec), anti-human CD8-PE/Vio770(Miltenyi Biotec)) and proliferation was measured by multicolor flow cytometry analysis using MACSQuant Analyzer 10 (Miltenyi Biotec). ScDb-scFv showed a 3-fold higher activity on CD8⁺ T-cell proliferation (EC₅₀=90±30 pM) and a 6-fold higher proliferation of CD4⁺ T-cells (EC₅₀=80±20 pM) compared to the bivalent scDb (EC₅₀=300±70 pM for CD8⁺ T-cell proliferation; EC₅₀=500±100 pM for CD4⁺ T-cell proliferation), respectively (FIG. 4C) (Table 2). Interestingly, no differences between scDb-scFv and scDb concerning activation of T-cell subpopulations was observed. Treatment with scDb-scFv and scDb mainly led to proliferation of central memory (T_{CM}) and effector memory (T_{EM}) CD4⁺ and CD8⁺ T-cells (FIG. 4D).

TABLE 2
Overview of T-cell activation mediated by scDb-
scFv and scDb molecules using MCF-7 cell line.

EC₅₀ [pM]

| | scDb-scFv | scDb |
|---|---|---|
| IL-2 | 403 ± 234 | 404 ± 147 |
| IFN-γ | 567 ± 456 | 609 ± 229 |
| CD4⁺CD69⁺ | 3 ± 3 | 100 ± 100 |
| CD8⁺CD69⁺ | 20 ± 20 | 160 ± 120 |
| Proliferation of CD4⁺ T-cells | 80 ± 20 | 500 ± 100 |
| Proliferation of CD8⁺ T-cells | 90 ± 30 | 300 ± 70 |

EC₅₀ values are shown in pM.
Mean ± SD, n = 3.

### Example 4: Target Cell Killing by a Trivalent, Bispecific scDb-scFv Fusion Protein Targeting HER3 and CD3

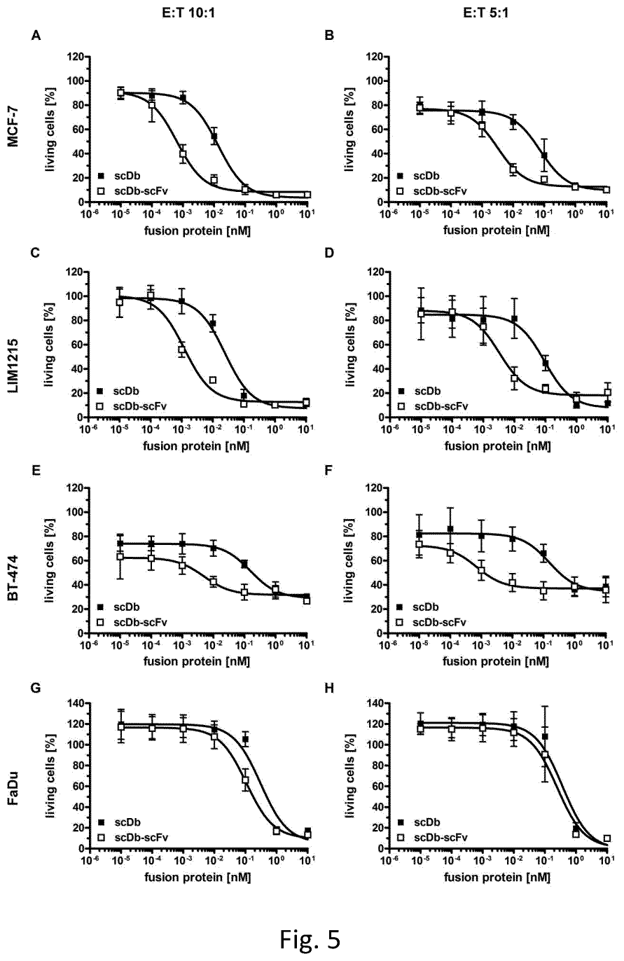

Cytotoxic effects of PBMCs on target cells mediated by scDb-scFv (SEQ ID NO: 6) were determined using HER3-positive cell lines with high (MCF-7: 17,283 HER3/cell; LIM1215: 19,877 HER3/cell), intermediate (BT-474: 11,244 HER3/cell) and low (FaDu: 2,884 HER3/cell) antigen expression. Previously seeded target cells (2×10⁴ cells/well) were incubated with bispecific antibodies (bsAb) for 15 min at RT prior to addition of PBMCs (E:T ratios of 10:1 or 5:1). After incubation of 3 days at 37° C., supernatants were discarded and viable target cells were stained with crystal violet. Staining was solved in methanol (50 μl/well) and optical density measured at 550 nm using the Tecan spark (Tecan). Both, scDb-scFv (SEQ ID NO: 6) and scDb (SEQ ID NO: 5) were able to redirect unstimulated PBMCs to lyse HER3-expressing cancer cells in a concentration-dependent manner. The activity of scDb-scFv and scDb was evaluated by efficacy (maximum inhibitory effect) and potency (EC₅₀ value in cell killing). No significant difference between scDb-scFv and scDb was observed regarding efficacy (FIG. 5A-F). On MCF-7, LIM1215, and FaDu cells, bsAb treatment led to 80-100% killing of tumor cells. Interestingly, only 60-70% of BT-474 cells were killed upon bsAb treatment. In contrast, tremendous differences were observed regarding potency. Here, scDb-scFv showed superior potency compared to scDb using MCF-7 (˜14- to 20-fold), LIM1215 (˜26- to 30-fold), and BT-474 (˜28- to 85-fold) cell lines, respectively (Table 3). However, scDb-scFv was only 3-fold more potent in comparison to the scDb using an E:T ratio of 10:1 on the HER3-low expressing cell line FaDu. Decreasing the E:T ratio to 5:1 using the FaDu cell line resulted in a 2-fold increased potency of scDb-scFv compared to scDb.

TABLE 3
Overview of scDb-scFv and scDb mediated cytotoxicity of
PBMCs using different effector:target (E:T) ratios.

EC₅₀ [pM]

| E:T | cell line | Her3/cell | scDb-scFv | scDb |
|---|---|---|---|---|
| 10:1 | MCF-7 | 17,283 | 1 ± 0.3 | 14 ± 5 |
| | LIM1215 | 19,877 | 1 ± 0.4 | 30 ± 4 |
| | BT-474 | 11,244 | 7 ± 7 | 200 ± 20 |
| | FaDu | 2,884 | 100 ± 20 | 300 ± 30 |
| 5:1 | MCF-7 | 17,283 | 3 ± 1 | 70 ± 40 |
| | LIM1215 | 19,877 | 3 ± 4 | 80 ± 20 |
| | BT-474 | 11,244 | 2 ± 3 | 170 ± 90 |
| | FaDu | 2,884 | 200 ± 100 | 400 ± 200 |

EC₅₀ values are shown in pM.
Mean ± SD, n = 3.

### Example 5: Production of scDb/scFv-Fc and scDb/Fab-Fc Fusion Proteins Targeting HER3 and CD3

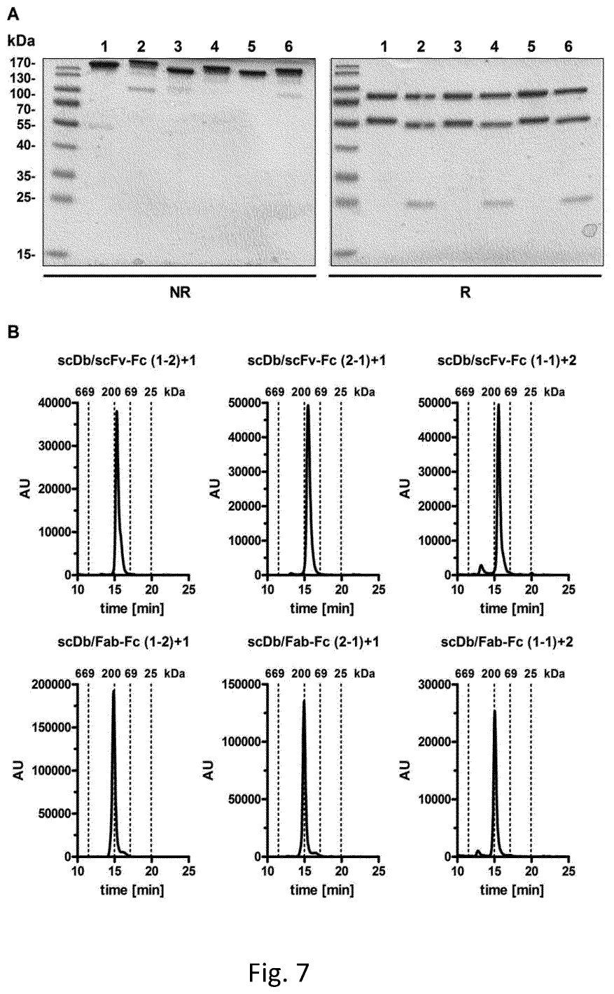

Trivalent, bispecific anti-HER3×anti-CD3 antibodies were generated by combining a scDb molecule either bispecific for HER3 (3-43) (Schmitt et al., 2017, mAbs, 9:831-843) and CD3 (huU3, humanized version of UCHT1), or monospecific for HER3 (HER3×HER3) with a scFv or Fab fragment specific for HER3 or CD3 by using a heterodimerizing Fc part (knob-into-hole technology) (Merchant et al., 1998, Nat Biotechnol. 16: 677-681) (FIG. 6). All trivalent bispecific antibodies were produced in transiently transfected HEK293-6E cells using polyethylenimine as transfection reagent. Two different plasmids were co-transfected for the scDb/scFv-Fc molecules ((SEQ ID NO: 7+9); (SEQ ID NO: 8+9); (SEQ ID NO: 12+13)), while three different plasmids were co-transfected for the scDb/Fab-Fc molecules ((SEQ ID NO: 7+10+11); (SEQ ID NO: 8+10+11); (SEQ ID NO: 12+14+15)). Proteins secreted into the cell culture supernatant were purified using FcXL CaptureSelect™ Affinity Matrix (Thermo Fisher Scientific) (scDb/scFv-Fc) or CaptureSelect™ IgG-CH1 Affinity Matrix (scDb/Fab-Fc) and a preparative size-exclusion FPLC on a Superdex 200 10/300 GL column (PBS as mobile phase, 0.5 ml/min flow rate). SDS-PAGE analysis of scDb/scFv-Fc revealed two bands under reducing conditions at approximately 55 kDa (scFv-Fc_{knob}) and 90 kDa (scDb-Fcₕₒₗₑ). The scDb/Fab-Fc molecules revealed three bands under reducing conditions at approximately 20 kDa (V_{L}-C_{L}), 55 kDa (V_{H}-C_{H}1-Fc_{knob}) and 90 kDa (scDb-Fcₕₒₗₑ) (FIG. 7A, right panel). Under non-reducing conditions, one major band at approximately 130 kDa (scDb/scFv-Fc) and 150 kDa (scDb/Fab-Fc) were observed (FIG. 7A, left panel) corresponding most likely to the dimer-assembled intact bispecific, trivalent molecules. Purity, integrity, and homogeneity of the trivalent, bispecific antibodies was confirmed using size-exclusion chromatography, where all proteins eluted as one major peak (FIG. 7B). One minor fraction of multimers was observed for both (1−1)+2 trivalent, bispecific antibodies (FIG. 7B, right panel).

### Example 6: Cell Binding of Trivalent, Bispecific Fc Fusion Proteins Targeting HER3 and CD3

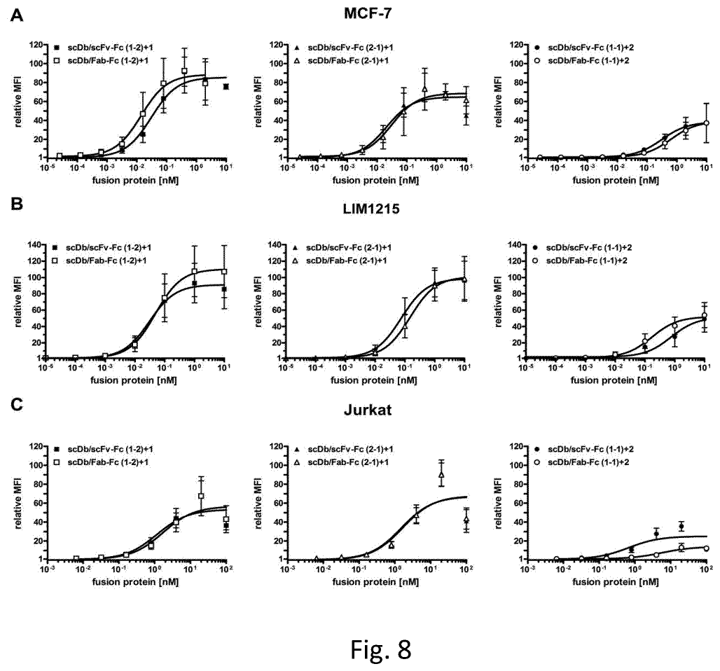

Binding of trivalent, bispecific fusion proteins to HER3-expressing (LIM1215, MCF-7) and CD3-expressing (Jurkat) cell lines was analyzed by flow cytometry. 2×10⁵ cells/well were incubated with a serial dilution of trivalent, bispecific antibodies for 1 h at 4° C. followed by detection using a PE-conjugated anti-human Fc antibody (Jackson ImmunoResearch Laboratories Inc). All trivalent, bispecific antibodies showed binding to HER3- and CD3-expressing target cells in a concentration-dependent manner. Regarding the HER3-expressing cell lines MCF-7 and LIM1215, the trivalent, bispecific antibodies in the (1−2)+1 and in the (2−1)+1 orientation bound with EC₅₀ values in the subnanomolar range ((SEQ ID NO: 7+9); (SEQ ID NO: 7+10+11); (SEQ ID NO: 8+9); (SEQ ID NO: 8+10+11)), whereby the trivalent, bispecific antibodies in the (1-1)+2 geometry ((SEQ ID NO: 12+13); (SEQ ID NO: 12+14+15)) showed lower fluorescence signals and reduced binding (FIGS. 8A and 8B) (Table 4). Concerning binding to the CD3-expressing cell line Jurkat, the trivalent, bispecific antibodies in the (1-2)+1 and in the (2−1)+1 orientation bound with EC₅₀ values in the low nanomolar range. A lower fluorescence signal was observed for the trivalent, bispecific antibodies in the (1−1)+2 orientation (FIG. 8C) (Table 4). Reduced binding to Jurkat cells was observed for the scDb/Fab-Fc molecule in the (1−1)+2 orientation.

TABLE 4
Overview of cell binding of trivalent, bispecific antibodies.

| | | EC₅₀ [pM] | | | | | |
|---|---|---|---|---|---|---|---|
| | | scDb/scFv-Fc | scDb/Fab-Fc | scDb/scFv-Fc | scDb/Fab-Fc | scDb/scFv-Fc | scDb/Fab-Fc |
| cell line | HER3/cell | (1 − 2) + 1 | (1 − 2) + 1 | (2 − 1) + 1 | (2 − 1) + 1 | (1 − 1) + 2 | (1 − 1) + 2 |
| MCF-7 | 17,283 | 40 ± 5 | 20 ± 9 | 30 ± 4 | 100 ± 100 | 400 ± 300 | 700 ± 300 |
| LIM1215 | 19,877 | 31 ± 7 | 53 ± 13 | 82 ± 46 | 173 ± 115 | 882 ± 588 | 249 ± 194 |
| Jurkat | — | 1,300 ± 500 | 2,300 ± 900 | 1,500 ± 100 | 1,700 ± 200 | 700 ± 200 | 8,700 ± 6,900 |

EC₅₀ values are shown in pM.
Mean ± SD, n = 3.

### Example 7: Activity of Trivalent, Bispecific Fc Fusion Proteins Targeting HER3 and CD3 on T-Cell Proliferation

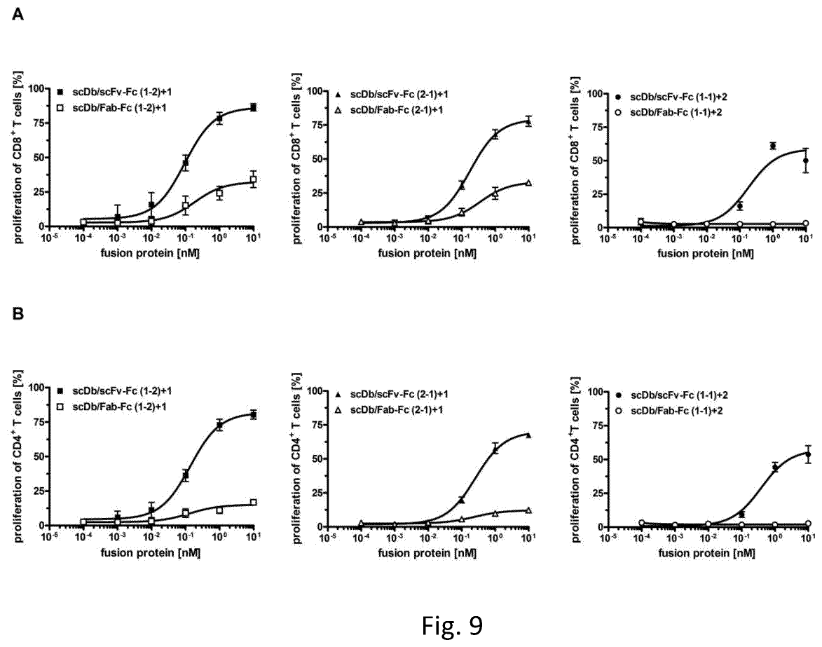

Proliferation of T-cells mediated by trivalent, bispecific Fc fusion proteins was determined in a co-culture assay using tumor cells (target) and human PBMCs (effector). Therefore, 2×10⁴ MCF-7 cells/well were incubated with fusion proteins for 15 min followed by the addition of CFSE-labeled PBMCs (2×10⁵ cells/well). PBMCs were harvested after 6 d of incubation at 37° C. and proliferation of CD4⁺ and CD8⁺ T-cells were identified by CFSE dilution in flow cytometry using MACSQuant Analyzer 10 (Miltenyi Biotec). All trivalent, bispecific antibodies in the scDb/scFv-Fc format ((SEQ ID NO: 7+9); (SEQ ID NO: 8+9); (SEQ ID NO: 12+13)) and scDb/Fab-Fc (1-2)+1 (SEQ ID NO: 8+10+11) and scDb/Fab (2-1)+1 (SEQ ID NO: 7+10+11) showed a concentration-dependent activation of CD8⁺(FIG. 9A) and CD4⁺(FIG. 9B) T-cell proliferation with EC₅₀ values in the subnanomolar range (Table 5). No activation of T-cells was observed for the trivalent, bispecific antibody in the scDb/Fab-Fc (1−1)+2 format (SEQ ID NO: 12+14+15).

TABLE 5
Overview of activation of T-cell proliferation by trivalent,
bispecific antibodies using MCF-7 cell line.

| | EC₅₀ [pM] | | | | | |
|---|---|---|---|---|---|---|
| | scDb/scFv-Fc | scDb/Fab-Fc | scDb/scFv-Fc | scDb/Fab-Fc | scDb/scFv-Fc | scDb/Fab-Fc |
| Proliferation | (1 − 2) + 1 | (1 − 2) + 1 | (2 − 1) + 1 | (2 − 1) + 1 | (1 − 1) + 2 | (1 − 1) + 2 |
| CD8⁺ T-cells | 97 ± 22 | 186 ± 118 | 181 ± 49 | 863 ± 523 | 191 ± 81 | n.d. |
| CD4⁺ T-cells | 140 ± 19 | 234 ± 285 | 265 ± 58 | 751 ± 795 | 393 ± 122 | n.d. |

EC₅₀ values are shown in pM.
Mean ± SD, n.d. = not determined, n = 3.

### Example 8: Target Cell Killing by Trivalent, Bispecific Antibodies Targeting HER3 and CD3

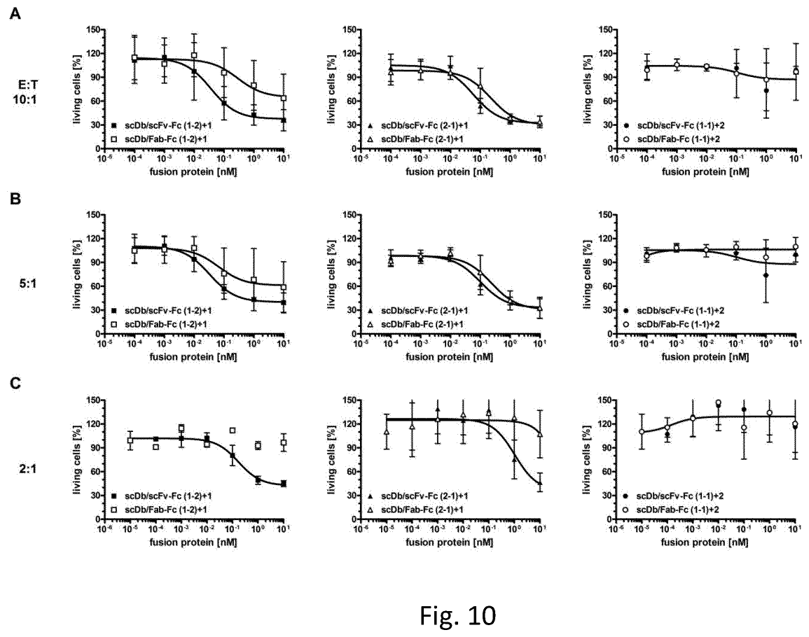

Cytotoxic effects of PBMCs on target cells mediated by trivalent, bispecific antibodies were determined using the HER3-positive cell line LIM1215 (19,877 Her3/cell). Target cells (2×10⁴ cells/well) were incubated with fusion proteins for 15 min at RT prior to addition of PBMCs (E:T ratios 10:1, 5:1, 2:1). After 3 days of incubation at 37° C., supernatants were discarded and viable target cells were stained with crystal violet. In E:T ratios of 10:1 and 5:1, the trivalent, bispecific antibodies in the (1-2)+1 ((SEQ ID NO: 8+9); (SEQ ID NO: 8+10+11)) and (2-1)+1 ((SEQ ID NO: 7+9); (SEQ ID NO: 7+10+11)) orientation mediated cancer cells lysis by T-cells in a concentration-dependent manner (FIG. 10). Additionally, an efficacy of ˜70% killing of tumor cells was observed for the trivalent, bispecific scDb/scFv-Fc molecules in the (1-2)+1 and (2-1)+1 orientation (FIG. 10A, B). Reducing the E:T ratio further to 2:1 led to an efficacy of ˜60% for the scDb/scFv-Fc molecules in the (1-2)+1 and (2-1)+1 orientation and very low target cell killing for the scDb/Fab-Fc molecules in the (1-2)+1 and (2-1)+1 orientation (FIG. 10C).

The scDb/scFv-Fc showed very strong (in the (1-2)+1 orientation) and strong (in the (2-1)+1 orientation) potency using 10:1 and 5:1 E:T ratio compared to the scDb/Fab-Fc molecules (FIGS. 10A & B, left and middle panels) (Table 6). In addition, using 2:1 (E:T) ratio, superior potency was observed for the scDb/scFv-Fc molecules in the (1-2)+1 and (2-1)+1 orientation compared to the scDb/Fab-Fc molecules (FIG. 10C, left and middle panels) (Table 6). The trivalent, bispecific antibodies in the (1-1)+2 ((SEQ ID NO: 12+13); (SEQ ID NO: 12+14+15)) orientation showed only marginal target cell killing by T-cells with an efficacy of only up to ˜10%.

TABLE 6
Overview of trivalent, bispecific antibodies-mediated cytotoxicity of PBMCs
(using different effector:target (E:T) ratios) using LIM1215 cell line.

| | EC₅₀ [pM] | | | | | |
|---|---|---|---|---|---|---|
| | scDb/scFv-Fc | scDb/Fab-Fc | scDb/scFv-Fc | scDb/Fab-Fc | scDb/scFv-Fc | scDb/Fab-Fc |
| E:T | (1 − 2) + 1 | (1 − 2) + 1 | (2 − 1) + 1 | (2 − 1) + 1 | (1 − 1) + 2 | (1 − 1) + 2 |
| 10:1 | 41 ± 35 | 1,079 ± 1,544 | 65 ± 30 | 238 ± 165 | n.d. | n.d. |
| 5:1 | 34 ± 12 | 2,003 ± 3,301 | 109 ± 50 | 260 ± 141 | n.d. | n.d. |
| 2:1 | 189 ± 124 | n.d. | 919 ± 479 | n.d. | n.d. | n.d. |

EC₅₀ values are shown in pM.
Mean ± SD, n.d. = not determined, n = 3.

### Example 9: Trivalent Bispecific scDb-scFv Fusion Proteins for Targeting EGFR-Expressing Tumor Cells

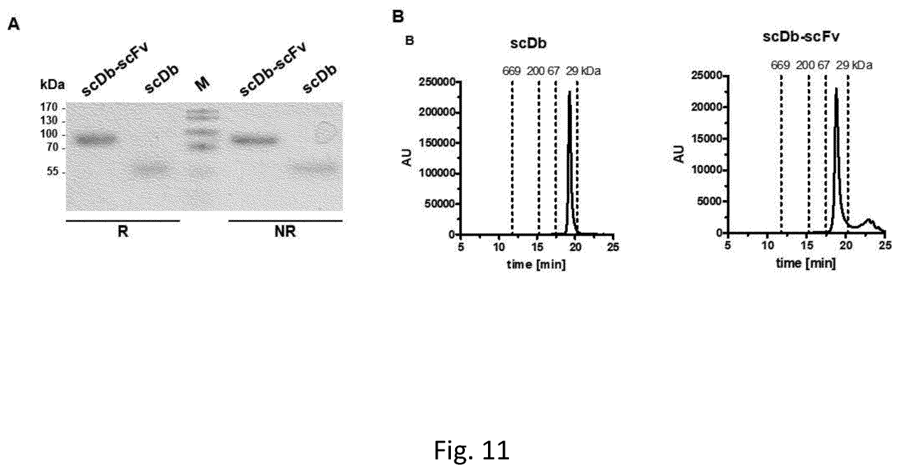

For the generation of the trivalent, bispecific bisAb (scDb-scFv) an anti-EGFR scFv was fused to the C-terminus of a bispecific scDb targeting CD3 and EGFR. The CD3 binding site is derived from a humanized version of the UCHT1 antibody, whereby the EGFR moiety consists of a humanized version of the EGFR-targeting antibody Cetuximab. The scDb and the scDb-scFv were produced in transiently transfected HEK293-6E cells (NRC Biotechnology Research Institute, Canada) using polyethylenimine (PEI; linear, 25 kDa, Sigma-Aldrich, 764604). 96 h post transfection, supernatants were harvested and scDb or scDb-scFv were purified by immobilized metal ion affinity chromatography (IMAC) followed by size-exclusion FPLC on a Superdex 200 10/300 GL column (PBS as mobile phase, 0.5 ml/min flow rate). Protein purity was analyzed using SDS-PAGE, where both proteins migrated according to their calculated molecular mass (scDb: 56.3 kDa, scDb-scFv 81.8 kDa) (FIG. 11A). Protein integrity was determined using Waters 2695 HPLC and a TSKgel SuperSW mAb HR column (Tosoh Bioscience) at a flow rate of 0.5 ml/min with 0.1 M Na₂HPO₄/NaH₂PO₄, 0.1 M Na₂SO₄, pH 6.7 as mobile phase. Thyroglobulin (669 kDa, Sr 8.5 nm), β-Amylase (200 kDa, Sr 5.4 nm), bovine serum albumin (67 kDa, Sr 3.55 nm) and carbonic anhydrase (29 kDa, Sr 2.35 nm) were used as reference proteins. All proteins eluted as one major peak in size-exclusion chromatography (FIG. 11B).

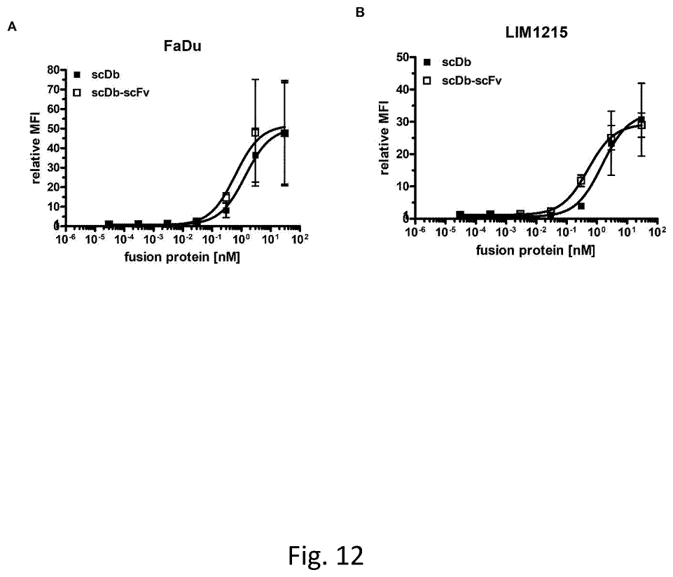

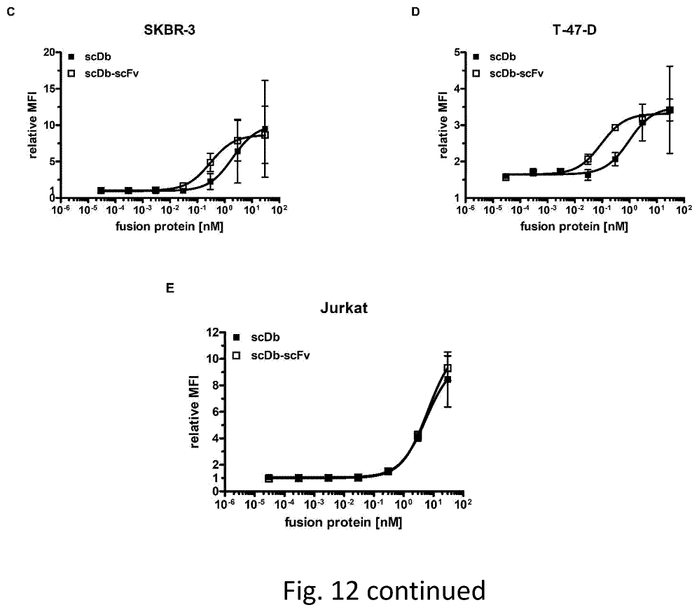

Binding analysis of scDb and scDb-scFv was performed by flow cytometry using tumor cell lines with different EGFR-expression levels (Table 7). Adherent cells were washed with PBS and shortly trypsinized at 37° C. Trypsin was quenched with FCS-containing medium and removed by centrifugation (500×g, 5 min.). 1×10⁵ target cells/well (FaDu, Lim1215, T-47-D and SKBR-3) were incubated with a serial dilution of scDb or scDb-scFv for 1 h at 4° C. After removing excess of recombinant protein by washing with PBA (PBS, 2% FBS, and 0.02% sodium azide), bound protein was detected using PE-conjugated anti-hexahistidyl tag mAb (Miltenyi Biotec). Fluorescence was measured using MACSquant Analyzer 10 (Miltenyi Biotec) and data were analyzed using FlowJo (Tree Star). Relative median fluorescence intensities (MFI) were calculated as followed: relative MFI=((MFIₛₐₘₚₗₑ-(MFI_{detection}-MFI_{cells}))/MFI_{cells}). Superior binding properties were observed for the trivalent, bispecific scDb-scFv compared to the bivalent, bispecific scDb. Subnanomolar EC₅₀ values were observed for the scDb-scFv, whereas the scDb showed 3-12 fold lower binding capacity, i.e. higher EC₅₀ values (FIG. 12, Table 7). On the CD3 expressing Jurkat cell lines, both the scDb and the scDb-scFv showed similar binding with EC₅₀ values of 5.4±2.0 nM and 6.4±0.02 nM, respectively.

TABLE 7
Overview of target cell binding by scDb-scFv and scDb molecules.

EC₅₀ [nM]

| cell line | EGFR expression | | scDb-scFv | scDb |
|---|---|---|---|---|
| FaDu | 143,250 | EGFR/cell | 0.5 ± 0.4 | 1.4 ± 1.4 |
| LIM1215 | 35.811 | EGFR/cell | 0.5 ± 0.2 | 1.6 ± 0.4 |
| T-47-D | 1,328 | EGFR/cell | 0.09 ± 0.005 | 1.1 ± 0.8 |
| SKBR-3 | 29,806 | EGFR/cell | 0.3 ± 0.2 | 2.3 ± 2.0 |

| | | | |
|---|---|---|---|
| Jurkat | — | 6.4 ± 0.02 | 5.4 ± 2.0 |

EC₅₀ values are shown in nM.
Mean ± SD, n = 3.

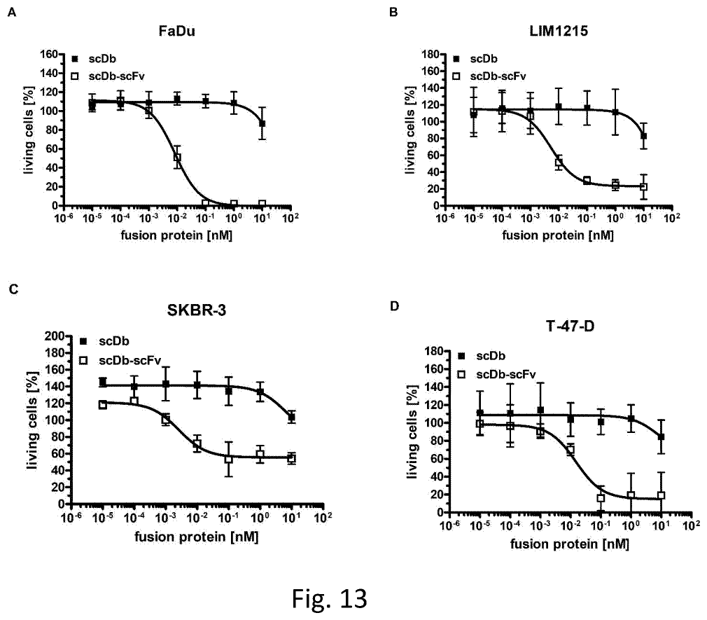

Cytotoxic effects of PBMCs on cancer cell lines mediated by the scDb and scDb-scFv was determined using EGFR-positive cell lines with high (FaDu: 143,250 EGFR/cell), intermediate (LIM1215: 35.811 EGFR/cell, SKBR-3: 29,806 EGFR/cell) and low (T-47-D: 1,328 EGFR/cell) target expression. A serial dilution of scDb and scDb-scFv was incubated on previously seeded target cells (2×10⁴ cells/well) followed by the addition of PBMCs (2×10⁵ cells/well) in an effector to target cell ratio of 10:1. After incubation of 3 days at 37° C., supernatants were discarded and viable target cells were stained with crystal violet. Staining was solved in methanol (50 μl/well) and optical density measured at 550 nm using the Tecan spark (Tecan) (FIG. 13) (Table 8). The activity of the scDb-scFv and scDb was evaluated in terms of efficacy (maximum inhibitory effect) and potency (EC₅₀ value in cell killing). Interestingly, only the scDb-scFv was able to redirect unstimulated PBMCs to lyse EGFR-expressing cancer cells in a concentration-dependent manner. Similar potency of the scDb-scFv was observed on the high and intermediate EGFR-expressing cell lines FaDu, LIM1215 and SKBR-3 with EC₅₀ values in the picomolar range. However, on the T-47-D cells with lowest EGFR-expression (1,328 EGFR/cell), scDb-scFv showed 10-fold lower potency compared to the high and intermediate expressing cancer cell lines. Regarding efficacy, similar maximum inhibitory effects of the scDb-scFv were observed on FaDu, T-47-D and LIM1215 cell lines with 80-90% tumor cell killing. Interestingly, only 60-70% of SKBR-3 cells were killed upon scDb-scFv treatment.

TABLE 8
Overview of scDb-scFv and scDb
mediated cytotoxicity of PBMCs.

EC₅₀ [nM]

| cell line | EGFR expression | | scDb-scFv | scDb |
|---|---|---|---|---|
| FaDu | 143,250 | EGFR/cell | 0.009 ± 0.002 | >10 |
| LIM1215 | 35.811 | EGFR/cell | 0.007 ± 0.002 | >10 |
| T-47-D | 1,328 | EGFR/cell | 0.09 ± 0.005 | >10 |
| SKBR-3 | 29,806 | EGFR/cell | 0.002 ± 0.001 | >10 |

EC₅₀ values are shown in nM.
Mean ± SD, n = 3.

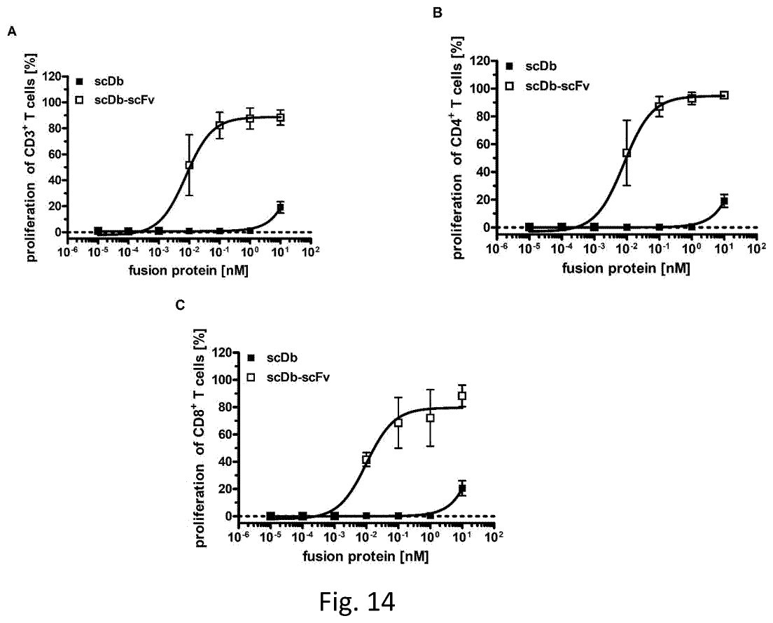

Effects on T-cell proliferation mediated by scDb-scFv and scDb was addressed in a coculture assay of tumor cell and effector cells (FIG. 14). Therefore, PBMCs were labeled with carboxyfluorescein diacetate succinimidyl ester (CFSE, ThermoFisher) at 625 nM/1×10⁶ cells/ml following the manufacturer's instructions. Then, previously seeded 2×10⁴ FaDu cells/well were incubated with a serial dilution of scDb or scDb-scFv for 15 min at RT followed by the addition of the CFSE-labeled PBMCs (2×10⁵ PBMCs/well). After 6 days of incubation at 37° C., cells were harvested and fluorescence-conjugated antibodies directed against respective cell surface markers (PerCP/Cy5.5 anti-human CD3 (Biolegend), anti-human CD4-VioBlue (Miltenyi Biotec), anti-human CD8-PE (Biolegend)) were used for labeling immune cells of interest. T-cell proliferation was determined by multicolor flow cytometry analysis using MACSQuant Analyzer 10 (Miltenyi Biotec). While only very low activity on T-cell proliferation was observed for the scDb, scDb-scFv showed strong effects on T-cell proliferation with an EC₅₀ value in the subnanomolar range. Interestingly, similar activation of proliferation of all investigated T-cell types was observed for the scDb-scFv.

### Example 10: Trivalent, Bispecific scDb/scFv-Fc and scDb/Fab-Fc Fusion Proteins Targeting CEA and CD3

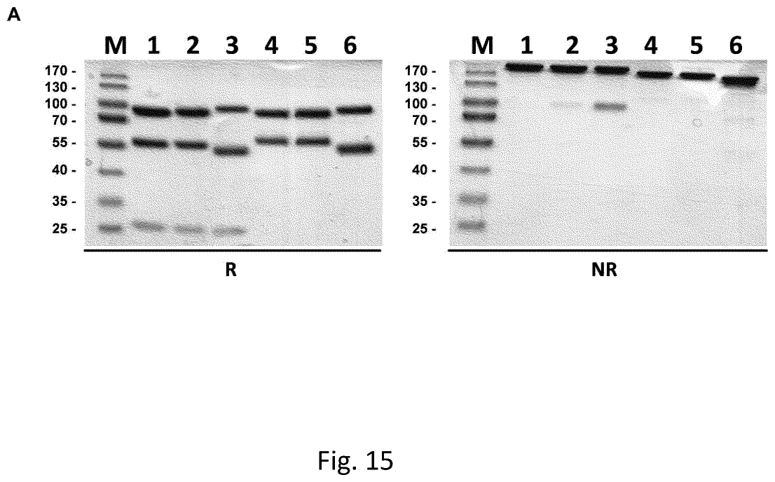

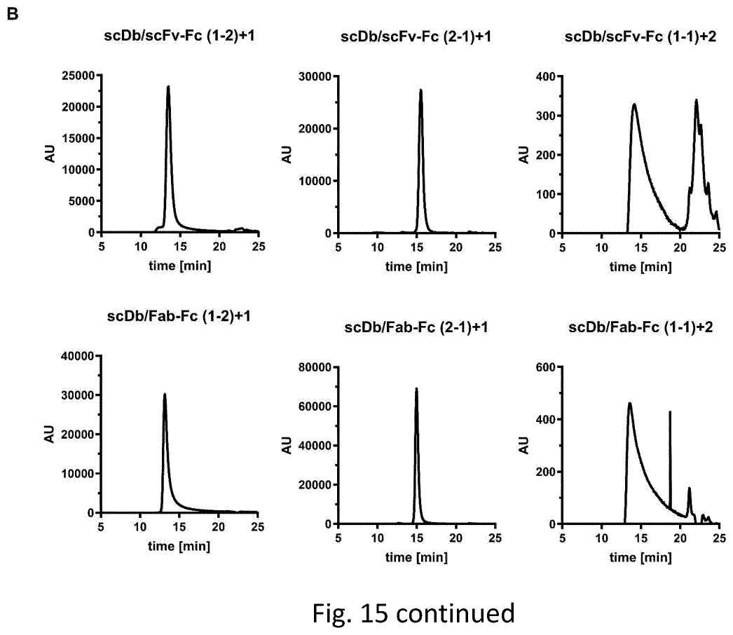

Trivalent, bispecific anti-CEAxanti-CD3 antibodies were generated by combining a scDb molecule either bispecific for CEA (1, Müller et al., 2007, J Biol Chem, 282: 12650-12660) and CD3 (2, huU3, humanized version of UCHT1), or monospecific for CEA (CEA×CEA) with a scFv or Fab fragment specific for CEA or CD3 by using a heterodimerizing Fc part (knob-into-hole technology) (see FIG. 6 for an overview of formats). All trivalent, bispecific antibodies were produced in transiently transfected HEK293-6E cells using polyethylenimine as transfection reagent. Two different plasmids were co-transfected for the scDb/scFv-Fc molecules ((scDb/scFv-Fc (1-2)+1, SEQ ID NO: 20+22); scDb/scFv-Fc (2-1)+1, (SEQ ID NO: 21+22); scDb/scFv-Fc (1-1)+2, (SEQ ID NO: 13+25)), while three different plasmids were co-transfected for the scDb/Fab-Fc molecules ((scDb/Fab-Fc (1-2)+1, SEQ ID NO: 20+23+24); scDb/Fab-Fc (2-1)+1, (SEQ ID NO: 21+23+24); scDb/Fab-Fc (1-1)+2, (SEQ ID NO: 14+15+25)). Proteins secreted into the cell culture supernatant were purified using FcXL CaptureSelect™ Affinity Matrix (Thermo Fisher Scientific) (scDb/scFv-Fc) or CaptureSelect™ IgG-CH1 Affinity Matrix (scDb/Fab-Fc) and a preparative size-exclusion FPLC on a Superdex 200 10/300 GL column (PBS as mobile phase, 0.5 ml/min flow rate). SDS-PAGE analysis of scDb/scFv-Fc revealed two bands under reducing conditions at approximately 55 kDa (scFv-Fc_{knob}) and 90 kDa (scDb-Fcₕₒₗₑ). The scDb/Fab-Fc molecules revealed three bands under reducing conditions at approximately 27 kDa (V_{L}-C_{L}), 55 kDa (V_{H}-C_{H}1-Fc_{knob}) and 90 kDa (scDb-Fcₕₒₗₑ) (FIG. 15A, left panel). Under non-reducing conditions, one major band at approximately 150 kDa (scDb/scFv-Fc) and 180 kDa (scDb/Fab-Fc) were observed (FIG. 15A, right panel) corresponding to the dimer-assembled intact trivalent, bispecific molecules. Purity, integrity, and homogeneity of the trivalent, bispecific antibodies was confirmed using size-exclusion chromatography, where all proteins eluted as one major peak (FIG. 15B). Of note, the trivalent scDb/scFv-Fc and scDb/Fab-Fc molecules in the configuration of (1-1)+2 were not detected in the correct time of retention in size-exclusion chromatography. Therefore, these two molecules were excluded from our additional experiments using the anti-CEAxanti-CD3 molecules in cytotoxicity and immunostimulatory assays.

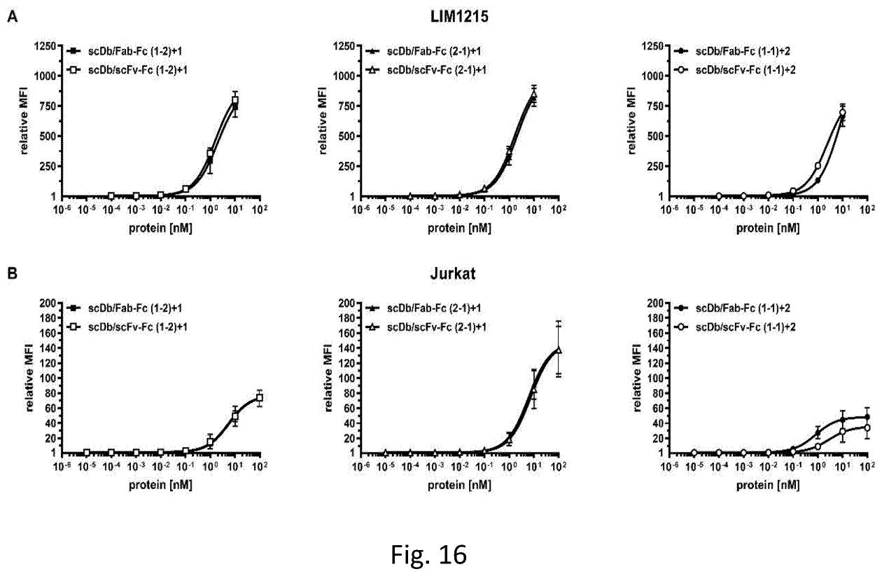

Binding of trivalent, bispecific fusion proteins to CEA-expressing (LIM1215) and CD3-expressing (Jurkat) cell lines was analyzed by flow cytometry. 2×10⁵ cells/well were incubated with a serial dilution of trivalent, bispecific antibodies for 1 h at 4° C. followed by detection using a PE-conjugated anti-human Fc antibody (Jackson ImmunoResearch Laboratories Inc). All trivalent, bispecific antibodies showed binding to CEA- and CD3-expressing target cells in a concentration-dependent manner. Regarding the CEA-expressing cell line LIM1215, the trivalent, bispecific antibodies bound with EC₅₀ values in the nanomolar range (FIG. 16A) (Table 9). Concerning binding to the CD3-expressing cell line Jurkat, the trivalent, bispecific antibodies in the (1-2)+1 and in the (2-1)+1 configuration bound with EC₅₀ values in the low nanomolar range, while the antibodies in the (1-1)+2 configuration showed only reduced intensity (FIG. 16B).

TABLE 9
Overview of cell binding of trivalent, bispecific antibodies.

| | EC₅₀ [nM] | | | | | |
|---|---|---|---|---|---|---|
| | scDb/scFv-Fc | scDb/Fab-Fc | scDb/scFv-Fc | scDb/Fab-Fc | scDb/scFv-Fc | scDb/Fab-Fc |
| cell line | (1 − 2) + 1 | (1 − 2) + 1 | (2 − 1) + 1 | (2 − 1) + 1 | (1 − 1) + 2 | (1 − 1) + 2 |
| LIM1215 | 1.6 ± 0.1 | 2.5 ± 1.2 | 1.7 ± 0.1 | 2.1 ± 0.4 | 2.4 ± 0.3 | 8.5 ± 1.0 |
| Jurkat | 7.7 ± 3.3 | 5.7 ± 0.06 | 7.9 ± 1.6 | 6.1 ± 0.8 | 2.8 ± 1.5 | 0.8 ± 0.1 |

EC₅₀ values are shown in nM.
Mean ± SD, n = 3.

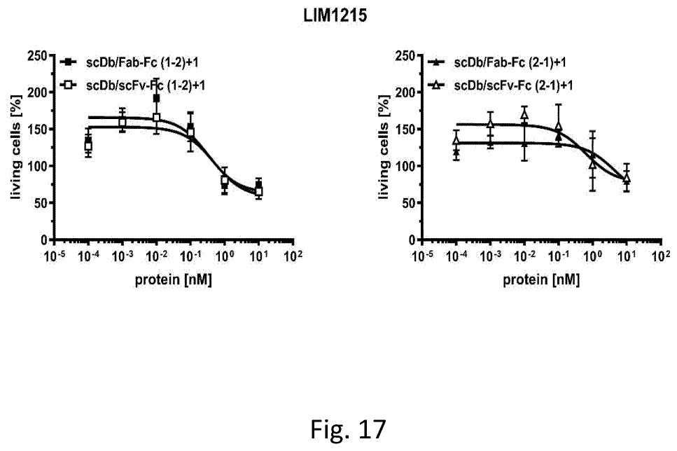

Cytotoxic effects of PBMCs on target cells mediated by trivalent, bispecific antibodies were determined using the CEA-positive cell line LIM1215. In this experiment, only the scDb/scFv-Fc (1-2)+1 or scDb/Fab-Fc (1-2)+1 and scDb/scFv-Fc (2-1)+1 or scDb/Fab-Fc (2-1)+1 molecule were included, as the third configuration was produced in very low ranges. Target cells (2×10⁴ cells/well) were incubated with fusion proteins for 15 min at RT prior to addition of PBMCs (E:T ratios 10:1). After 3 days of incubation at 37° C., supernatants were discarded and viable target cells were stained with crystal violet. The trivalent, bispecific antibodies in the (1-2)+1 and (2-1)+1 configuration mediated cancer cells lysis by T-cells in a concentration-dependent manner (FIG. 17). EC₅₀ values were slightly decreased using molecules in the (1-2)+1 configuration compared to the molecules with the (2-1)+1 configuration (FIG. 17) (Table 10).

TABLE 10
Overview of trivalent, bispecific antibodies-mediated
cytotoxicity of PBMCs using LIM1215 cell line.

| | EC₅₀ [nM] | | | |
|---|---|---|---|---|
| | scDb/scFv-Fc | scDb/Fab-Fc | scDb/scFv-Fc | scDb/Fab-Fc |
| cell line | (1 − 2) + 1 | (1 − 2) + 1 | (2 − 1) + 1 | (2 − 1) + 1 |
| LIM1215 | 0.5 ± 0.3 | 0.4 ± 0.09 | 0.3 ± 0.2 | 1.2 ± 0.4 |

EC₅₀ values are shown in nM.
Mean ± SD, n.d. = not determined, n = 3.

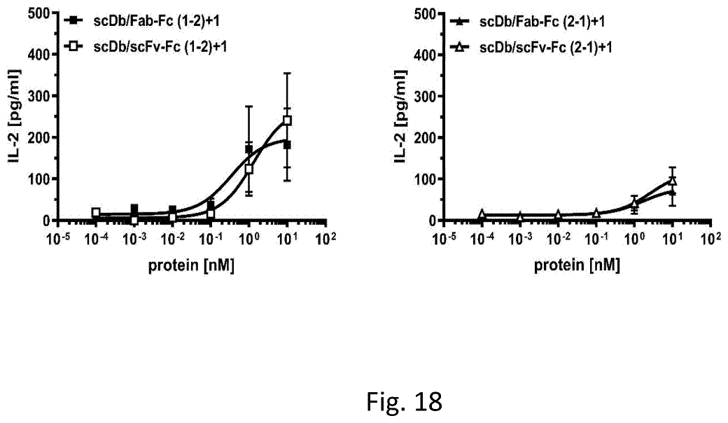

To address simultaneous binding of the trivalent, bispecific antibodies to tumor and effector cells, we investigated the activation of T-cells in a co-culture assay by measuring IL-2 levels. In this experiment, only the trivalent molecule in the (1-2)+1 and (2-1)+1 configuration were included. 2×10⁴ LIM1215 cells/well were incubated with a serial dilution of scDb/scFv-Fc and scDb/Fab-Fc fusion proteins for 15 min at RT followed by addition of 2×10⁵ PBMCs/well. After 24 h of incubation at 37° C., cell-free supernatants of the co-cultures were harvested and concentrations of IL-2 were determined using DuoSet sandwich ELISA kit (R&D Systems). All trivalent, bispecific antibodies showed a concentration-dependent cytokine release by T-cells (FIG. 18) (Table 11). However, the scDb/Fab-Fc (1-2)+1 and scDb/scFv-Fc (1-2)+1 showed lowest EC₅₀ values of IL-2 release and highest concentration of secreted IL-2 in these experiments.

TABLE 11
Overview of T-cell activation mediated by trivalent,
bispecific antibodies using LIM1215 cell line.

| | EC₅₀ [nM] | | | |
|---|---|---|---|---|
| | scDb/scFv-Fc | scDb/Fab-Fc | scDb/scFv-Fc | scDb/Fab-Fc |
| cell line | (1 − 2) + 1 | (1 − 2) + 1 | (2 − 1) + 1 | (2 − 1) + 1 |
| IL-2 | 1.4 ± 0.5 | 0.5 ± 0.2 | 1.4 ± 1.0 | 3.5 ± 0.4 |

EC₅₀ values are shown in nM.
Mean ± SD, n = 3.

### Example 11: Trivalent, Bispecific Fc Fusion Proteins Targeting EGFR and CD3

For the generation of the trivalent, bispecific anti-EGFRxanti-CD3 antibodies, a scDb molecule either bispecific for EGFR (1, hu225, humanized version of Cetuximab) and CD3 (2, huU3, humanized version of UCHT1), or monospecific for EGFR was combined with a scFv- or Fab-fragment specific for EGFR or CD3 by using a heterodimerizing Fc part (see FIG. 6 for an overview of formats). Thus, the CD3 binding site is either positioned in the scDb moiety or the Fab or scFv moiety.

All trivalent, bispecific antibodies were produced in transient transfection of HEK293-6E cells using polyethylenimine as transfection reagent. Co-transfections were performed by administration of two different plasmids for the scDb/scFv-Fc molecules ((scDb/scFv-Fc (1-2)+1, SEQ ID NO: 26+28); scDb/scFv-Fc (2-1)+1, (SEQ ID NO: 27+28); scDb/scFv-Fc (1-1)+2, (SEQ ID NO: 13+31)), while three different plasmids were co-transfected for the scDb/Fab-Fc molecules ((scDb/Fab-Fc (1-2)+1, SEQ ID NO: 26+29+30); scDb/Fab-Fc (2-1)+1, (SEQ ID NO: 27+29+30); scDb/Fab-Fc (1-1)+1, (SEQ ID NO: 14+15+31)). 96 h post transfection, supernatants were harvested and trivalent, bispecific molecules were purified with protein A, followed by size-exclusion FPLC on a Superdex 200 10/300 GL column (PBS as mobile phase, 0.5 ml/min flow rate).

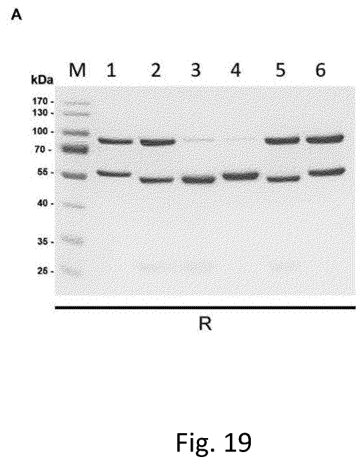

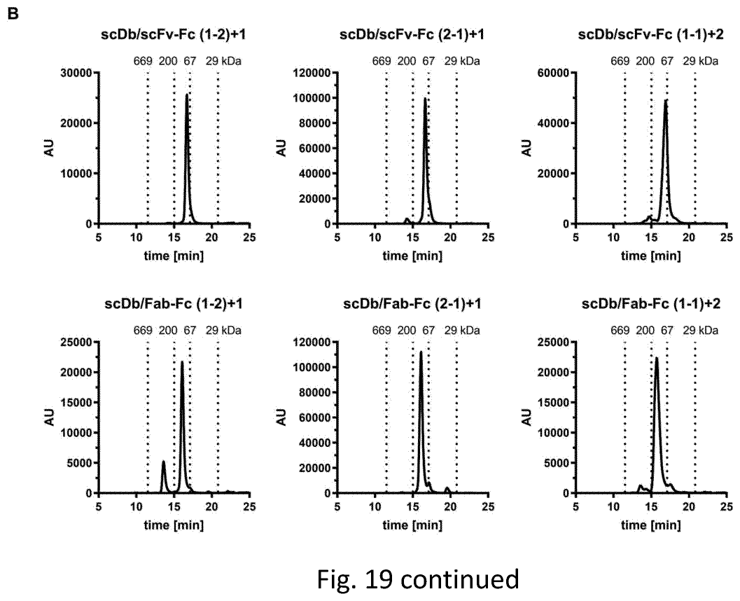

Protein purity was analyzed using SDS-PAGE analysis. Here, the scDb/scFv-Fc revealed two bands under reducing conditions at approximately 55 kDa (scFv-Fc_{knob}) and 90 kDa (scDb-Fcₕₒₗₑ). The scDb/Fab-Fc molecules revealed three bands under reducing conditions at approximately 27 kDa (V_{L}-C_{L}), 55 kDa (V_{H}-C_{H}1-Fc_{knob}) and 90 kDa (scDb-Fcₕₒₗₑ) (FIG. 19A). Of note, the scDb-Fcₕₒₗₑ chain only showed very low expression for the molecules in the (1-1)+2 configuration.

Purity, integrity, and homogeneity of the trivalent, bispecific antibodies was confirmed using Waters 2695 HPLC and a TSKgel SuperSW mAb HR column (Tosoh Bioscience) at a flow rate of 0.5 ml/min with 0.1 M Na₂HPO₄/NaH₂PO₄, 0.1 M Na₂SO₄, pH 6.7 as mobile phase. Thyroglobulin (669 kDa, Sr 8.5 nm), β-Amylase (200 kDa, Sr 5.4 nm), bovine serum albumin (67 kDa, Sr 3.55 nm) and carbonic anhydrase (29 kDa, Sr 2.35 nm) were used as reference proteins. In size-exclusion chromatography, all proteins eluted as one major peak (FIG. 19B). One minor peak of multimers was observed for the scDb/Fab-Fc (1-2)+1 (FIG. 19B, left panel, bottom).

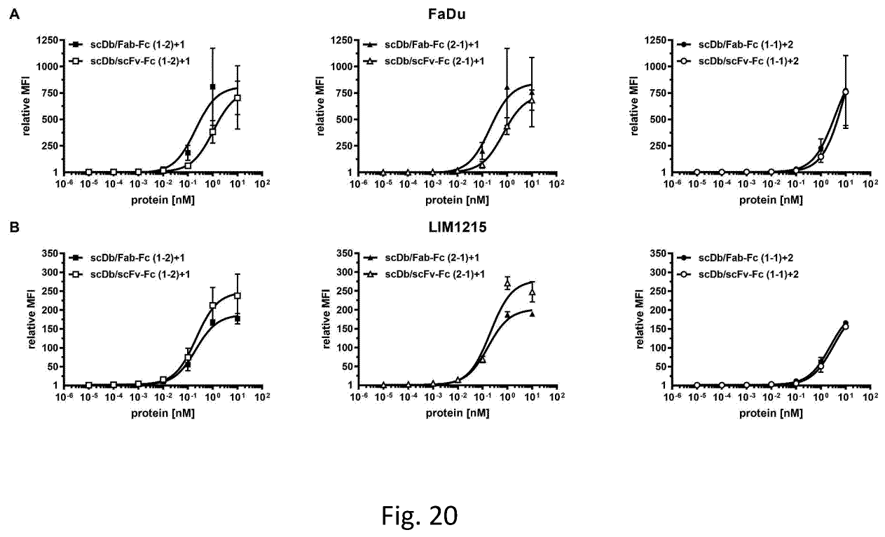

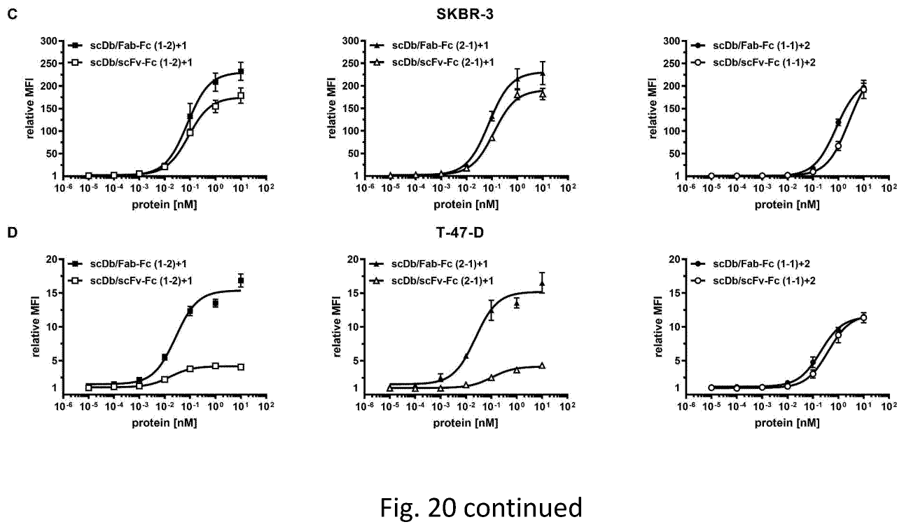

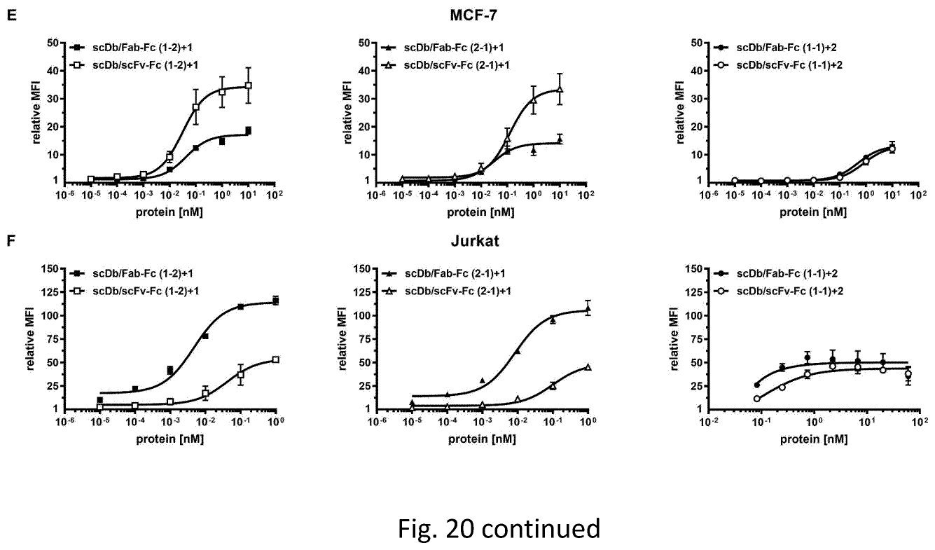

Binding analysis of the trivalent, bispecific antibodies was performed by flow cytometry using tumor cell lines with different EGFR-expression levels (FIG. 20) (Table 12). Adherent cells were washed with PBS and shortly trypsinized at 37° C. Trypsin was quenched with FCS-containing medium and removed by centrifugation (500×g, 5 min.). Target cells (FaDu, LIM1215, SKBR-3, T-47-D, MCF-7 and Jurkat cells) at 1×10⁵ cells/well were incubated with a serial dilution of trivalent, bispecific antibodies for 1 h at 4° C. After removing excess of recombinant protein by washing with PBA (PBS, 2% FBS, and 0.02% sodium azide), bound protein was detected using PE-conjugated anti-human Fc mAb (Jackson ImmunoResearch Laboratories Inc). Fluorescence was measured using MACSquant VYB (Miltenyi Biotec) and data were analyzed using FlowJo (Tree Star). Relative median fluorescence intensities (MFI) were calculated as followed: relative MFI=((MFIₛₐₘₚₗₑ-(MFI_{detection}-MFI_{cells}))/MFI_{cells}). While the trivalent, bispecific antibodies in the (1-2)+1 and the (2-1)+1 configurations showed similar binding in the low nanomolar range on all tested cell lines, the trivalent, bispecific antibodies in the (1-1)+2 configuration showed a reduced binding (FIG. 20A-E). On the CD3 expressing Jurkat cell line, the scDb/Fab-Fc in the (1-2)+1 and (2-1)+1 configuration showed similar EC₅₀ values in the low nanomolar range (1.0±0.4 nM and 1.4±0.2 nM, respectively). In contrast, the two scDb/scFv-Fc molecules in the (1-2)+1 and (2-1)+1 configuration showed weaker binding with EC₅₀ values of 9.6±5.8 nM and 16.3±6.6 nM, respectively. The two antibodies in the (1-1)+2 configuration showed very strong binding to CD3, although with lower signal intensity (FIG. 20F).

TABLE 12
Overview of target cell binding by trivalent, bispecific molecules.

| | | EC₅₀ [nM] | | | | | |
|---|---|---|---|---|---|---|---|
| | | scDb/scFv-Fc | scDb/Fab-Fc | scDb/scFv-Fc | scDb/Fab-Fc | scDb/scFv-Fc | scDb/Fab-Fc |
| cell line | EGFR/cell | (1 − 2) + 1 | (1 − 2) + 1 | (2 − 1) + 1 | (2 − 1) + 1 | (1 − 1) + 2 | (1 − 1) + 2 |
| FaDu | 143,250 | 1.1 ± 0.4 | 0.2 ± 0.02 | 0.7 ± 0.1 | 0.2 ± 0.01 | 8.9 ± 3.6 | 3.6 ± 0.5 |
| LIM1215 | 35,811 | 0.2 ± 0.02 | 0.2 ± 0.05 | 0.2 ± 0.006 | 0.2 ± 0.02 | 4.0 ± 3.0 | 2.6 ± 1.0 |
| T-47-D | 1,328 | 0.02 ± 0.004 | 0.03 ± 0.002 | 0.1 ± 0.04 | 0.03 ± 0.01 | 0.4 ± 0.2 | 0.2 ± 0.06 |
| SKBR-3 | 29,806 | 0.09 ± 0.02 | 0.1 ± 0.02 | 0.1 ± 0.02 | 0.1 ± 0.01 | 2.8 ± 1.2 | 0.8 ± 0.2 |
| MCF-7 | >1,900 | 0.03 ± 0.007 | 0.04 ± 0.01 | 0.1 ± 0.03 | 0.03 ± 0.003 | 0.8 ± 0.3 | 0.5 ± 0.2 |
| Jurkat | — | 9.6 ± 5.8 | 1.0 ± 0.4 | 16.3 ± 6.6 | 1.4 ± 0.2 | n.d. | n.d. |

EC₅₀ values are shown in nM.
Mean ± SD, n = 3.

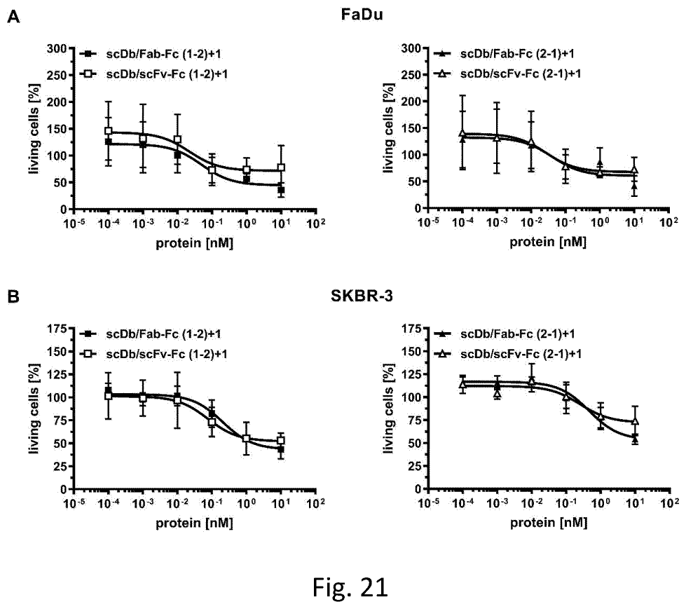

Cytotoxic effects of human PBMCs on cancer cell lines mediated by the trivalent, bispecific antibodies was determined using EGFR-positive cell lines with high (FaDu: 143,250 EGFR/cell) (FIG. 21A) and intermediate (SKBR-3: 29,806 EGFR/cell) (FIG. 21B) EGFR expression. In this study, we used only the scDb/scFv-Fc and scDb/Fab-Fc in the configuration (1-2)+1 and (2-1)+1, as the third configuration was not produced correctly. A serial dilution of trivalent bispecific antibodies was incubated on previously seeded target cells (2×10⁴ cells/well) followed by the addition of PBMCs (2×10⁵ cells/well) in an effector to target cell ratio of 5:1. After incubation of 3 days at 37° C., supernatants were discarded and viable target cells were stained with crystal violet. Staining was solved in methanol (50 μl/well) and optical density measured at 550 nm using the Tecan spark (Tecan) (FIG. 21) (Table 13). The activity of the trivalent bispecific antibodies was evaluated in terms of potency (EC₅₀ value in cell killing). All trivalent, bispecific antibodies were able to redirect unstimulated PBMCs to lyse EGFR-expressing cancer cells in a concentration-dependent manner. Similar potency was observed for EGFR-expressing cell lines FaDu and SKBR-3 with EC₅₀ values in the picomolar range (Table 13).

TABLE 13
Overview of trivalent, bispecific antibodies mediated cytotoxicity of PBMCs.

| | | EC₅₀ [pM] | | | |
|---|---|---|---|---|---|
| | | scDb/scFv-Fc | scDb/Fab-Fc | scDb/scFv-Fc | scDb/Fab-Fc |
| cell line | EGFR/cell | (1 − 2) + 1 | (1 − 2) + 1 | (2 − 1) + 1 | (2 − 1) + 1 |
| FaDu | 143,250 | 26 ± 8 | 50 ± 20 | 27 ± 8 | 56 ± 36 |
| SKBR-3 | 29,806 | 126 ± 41 | 209 ± 140 | 277 ± 270 | 209 ± 140 |

EC₅₀ values are shown in pM.
Mean, n = 3.

### Example 12: Trivalent, Trispecific Fc Fusion Proteins Targeting EGFR, HER3 and CD3

For the generation of the trivalent, trispecific anti-EGFRxanti-HER3×anti-CD3 antibodies a scDb molecule either bispecific for HER3 (1, 3-43) and CD3 (2, huU3, humanized version of UCHT1), or bispecific for HER3 (1, 3-43) and EGFR (3, hu225), was combined with a scFv- or Fab-fragment specific for CD3 or EGFR by using a heterodimerizing Fc part (knob-into-hole technology). All trivalent, trispecific antibodies produced in transient transfection of HEK293-6E cells using polyethylenimine as transfection reagent (see FIG. 6 for an overview of formats). Co-transfection was performed by administration of two different plasmids for the scDb/scFv-Fc molecules (scDb/scFv-Fc (1-2)+3 (SEQ ID NO: 7+28); scDb/scFv-Fc (2-1)+3 (SEQ ID NO: 8+28); scDb/scFv-Fc (1-3)+2 (SEQ ID NO: 13+32)), while three different plasmids were co-transfected for the scDb/Fab-Fc molecules (scDb/Fab-Fc (1-2)+3 (SEQ ID NO: 7+29+30); scDb/Fab-Fc (2-1)+3 (SEQ ID NO: 8+29+30); scDb/Fab-Fc (1-3)+2 (SEQ ID NO: 14+15+32)). 96 h post transfection, supernatants were harvested and trivalent, bispecific molecules were purified with protein A, followed by size-exclusion FPLC on a Superdex 200 10/300 GL column (PBS as mobile phase, 0.5 ml/min flow rate).

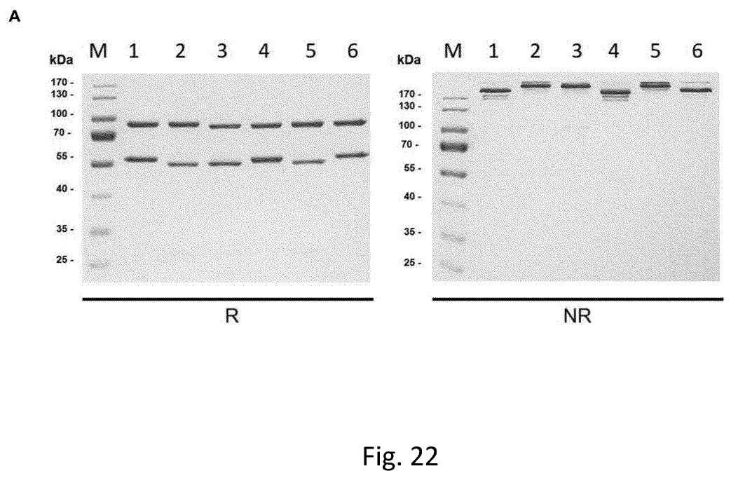

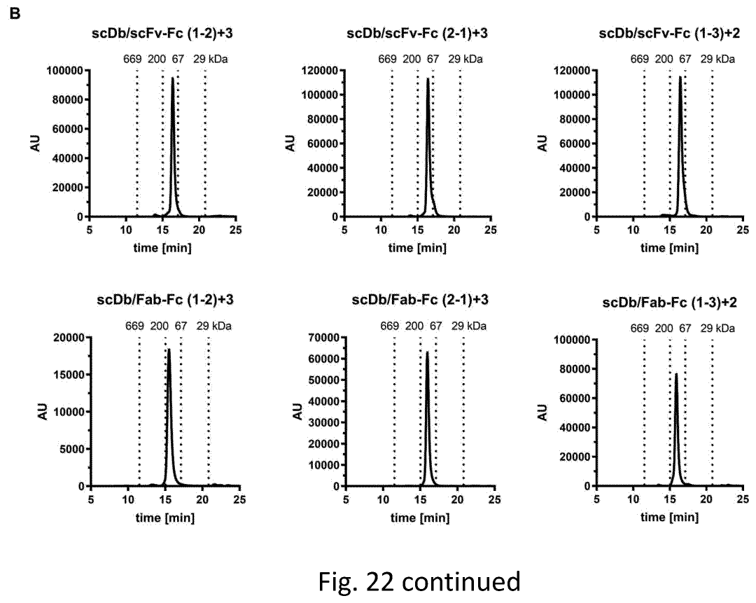

SDS-PAGE analysis of scDb/scFv-Fc revealed two bands under reducing conditions at approximately 55 kDa (scFv-Fc_{knob}) and 90 kDa (scDb-Fcₕₒₗₑ). The scDb/Fab-Fc molecules revealed three bands under reducing conditions at approximately 26 kDa (V_{L}-C_{L}), 55 kDa (V_{H}-C_{H}1-Fc_{knob}) and 90 kDa (scDb-Fcₕₒₗₑ) (FIG. 22A, left panel). Under non-reducing conditions, one major band at approximately >170 kDa were observed (FIG. 22A, right panel) corresponding to the dimer-assembled intact trispecific, trivalent molecules.

Purity, integrity, and homogeneity of the trivalent, trispecific antibodies was confirmed using Waters 2695 HPLC and a TSKgel SuperSW mAb HR column (Tosoh Bioscience) at a flow rate of 0.5 ml/min with 0.1 M Na₂HPO₄/NaH₂PO₄, 0.1 M Na₂SO₄, pH 6.7 as mobile phase. Thyroglobulin (669 kDa, Sr 8.5 nm), β-Amylase (200 kDa, Sr 5.4 nm), bovine serum albumin (67 kDa, Sr 3.55 nm) and carbonic anhydrase (29 kDa, Sr 2.35 nm) were used as reference proteins. In size exclusion chromatography, all proteins eluted as one major peak (FIG. 22B).

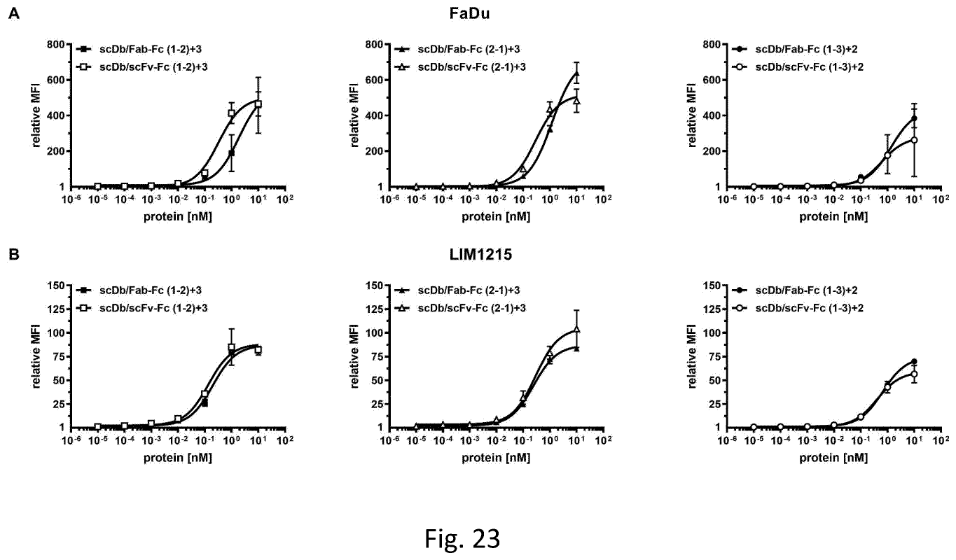

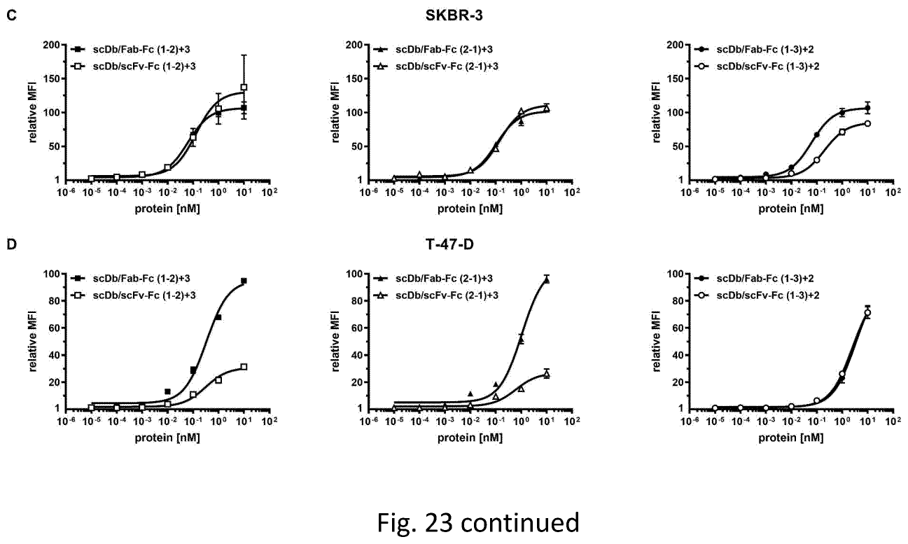

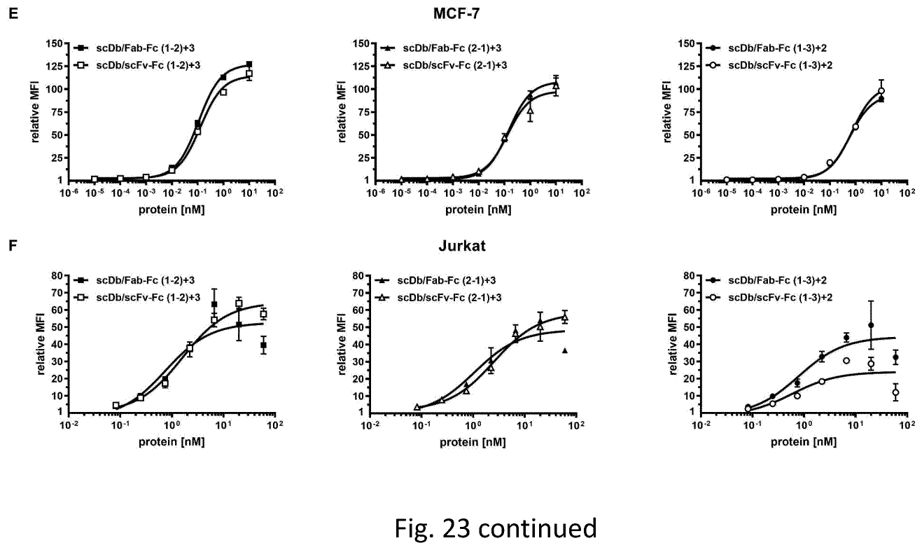

Trivalent, trispecific antibodies were analyzed for their binding to tumor cell lines with different EGFR- and HER3-expression levels (Table 14) by flow cytometry. Adherent cells were washed with PBS and shortly trypsinized at 37° C. Trypsin was quenched with FCS-containing medium and removed by centrifugation (500×g, 5 min.). 1×10⁵ target cells/well (FaDu, LIM1215, SKBR-3, T-47-D, MCF-7 and Jurkat cells) were incubated with a serial dilution of trivalent, trispecific antibodies for 1 h at 4° C. After removing excess of recombinant protein by washing with PBA (PBS, 2% FBS, and 0.02% sodium azide), bound protein was detected using PE-conjugated anti-human Fc mAb (Jackson ImmunoResearch Laboratories Inc). Fluorescence was measured using MACSquant VYB (Miltenyi Biotec) and data were analyzed using FlowJo (Tree Star). Relative median fluorescence intensities (MFI) were calculated as followed: relative MFI=((MFIₛₐₘₚₗₑ-(MFI_{detection}-MFI_{cells}))/MFI_{cells}). All trivalent, trispecific molecules showed similar binding on the SKBR-3 cell lines (FIG. 23C), while the (1-2)+3 and the (2-1)+3 configurations showed increased binding on the LIM1215 (FIG. 23B), T-47-D (FIG. 23D) and MCF-7 (FIG. 23E) cell lines. Interestingly, the trivalent, trispecific antibodies in the scDb/scFv-Fc conformation showed higher binding capacity compared to the scDb/Fab-Fc molecules on the FaDu cell line (FIG. 23A). On the CD3-expressing cell line Jurkat the antibodies in the (1-2)+3 and the (2-1)+3 configuration showed similar binding while the molecules in the (1-3)+2 configuration showed reduced EC₅₀ values (0.6 and 0.8 nM) with lowed signal intensity (FIG. 23F).

TABLE 14
Overview of target cell binding by trivalent, trispecific antibodies.

| | | | EC₅₀ [nM] | | | | | |
|---|---|---|---|---|---|---|---|---|
| | | | scDb/scFv-Fc | scDb/Fab-Fc | scDb/scFv-Fc | scDb/Fab-Fc | scDb/scFv-Fc | scDb/Fab-Fc |
| cell line | HER3/cell | EGFR/cell | (1 − 2) + 3 | (1 − 2) + 3 | (2 − 1) + 3 | (2 − 1) + 3 | (1 − 3) + 2 | (1 − 3) + 2 |
| FaDu | 2,884 | 143,250 | 0.3 | 1.9 | 0.3 | 1.2 | 0.6 | 1.3 |
| LIM1215 | 19,877 | 35,811 | 0.1 | 0.2 | 0.3 | 0.3 | 0.4 | 0.7 |
| T-47-D | 7,021 | 1,328 | 0.3 | 0.3 | 0.6 | 1.1 | 2.5 | 3.3 |
| SKBR-3 | 14,084 | 29,806 | 0.1 | 0.06 | 0.1 | 0.1 | 0.2 | 0.06 |
| MCF-7 | 17,283 | >1,900 | 0.1 | 0.1 | 0.1 | 0.2 | 0.7 | 0.6 |
| Jurkat | — | — | 1.6 | 0.8 | 2.6 | 1.1 | 0.6 | 0.8 |

EC₅₀ values are shown in nM.
Mean, n = 1.

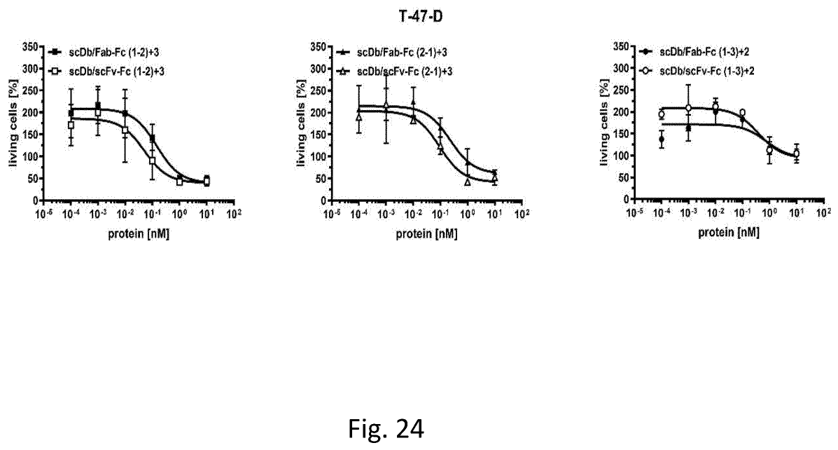

Cytotoxic effects of PBMCs on target cells mediated by trivalent, triispecific antibodies were determined using the EGFR- and HER3-positive cell lines (T-47-D). Target cells (2×10⁴ cells/well) were incubated with the different trivalent, trispecific antibodies for 15 min at RT prior to addition of PBMCs (E:T ratios 5:1). After 3 days of incubation at 37° C., supernatants were discarded and viable target cells were stained with crystal violet. All trivalent, trispecific antibodies mediated cancer cells lysis by T-cells in a concentration-dependent manner (FIG. 24). EC₅₀ values of scDb/scFv-Fc in the configuration of (1-2)+3 and (2-1)+3 were slightly decreased compared to the scDb/Fab-Fc molecules in the same configuration. Surprisingly, both molecules in the (1-3)+2 configuration showed lowest activity concerning EC₅₀ value and killing of the target cells (Table 15).

TABLE 15
Overview of trivalent, trispecific antibodies mediated cytotoxicity of PBMCs.

| | | | EC₅₀ [pM] | | | | | |
|---|---|---|---|---|---|---|---|---|
| | | | scDb/scFv-Fc | scDb/Fab-Fc | scDb/scFv-Fc | scDb/Fab-Fc | scDb/scFv-Fc | scDb/Fab-Fc |
| cell line | HER3/cell | EGFR/cell | (1 − 2) + 3 | (1 − 2) + 3 | (2 − 1) + 3 | (2 − 1) + 3 | (1 − 3) + 2 | (1 − 3) + 2 |
| T-47-D | 7,021 | 1,328 | 48 ± 42 | 147 ± 10 | 93 ± 4 | 228 ± 80 | 361 ± 164 | 962 ± 531 |

EC₅₀ values are shown in pM.
Mean, n = 2.

### Example 13: Cell Binding of Trivalent, Bispecific Fc Fusion Proteins Targeting FAP and CD3

Trivalent, bispecific Fc fusion proteins were generated combining a FAP-targeting binding site with a CD3-binding site in different arrangements. Trivalent, bispecific anti-FAPxanti-CD3 antibodies were generated by combining a scDb molecule either bispecific for FAP (1, hu36; Fabre et al., 2020, Clin. Cancer Res. 26, 3420-3430) and CD3 (2, huU3, humanized version of UCHT1), or monospecific for FAP (FAPxFAP) with a scFv or Fab fragment specific for FAP or CD3 by using a heterodimerizing Fc part (knob-into-hole technology) (see FIG. 6 for an overview of formats). All trivalent, bispecific antibodies were produced in transiently transfected HEK293-6E cells using polyethylenimine as transfection reagent. Two different plasmids were co-transfected for the scDb/scFv-Fc molecules ((scDb/scFv-Fc (1-2)+1, SEQ ID NO: 33+35); scDb/scFv-Fc (2-1)+1, (SEQ ID NO: 34+35); scDb/scFv-Fc (1-1)+2 (SEQ ID NO: 13+38)), while three different plasmids were co-transfected for the scDb/Fab-Fc molecules ((scDb/Fab-Fc (1-2)+1, SEQ ID NO: 33+36+37); scDb/Fab-Fc (2-1)+1, (SEQ ID NO: 34+36+37); scDb/Fab-Fc (1-1)+2, (SEQ ID NO: 14+15+38)). Proteins secreted into the cell culture supernatant were purified using protein A and a preparative size-exclusion FPLC on a Superdex 200 10/300 GL column (PBS as mobile phase, 0.5 ml/min flow rate).

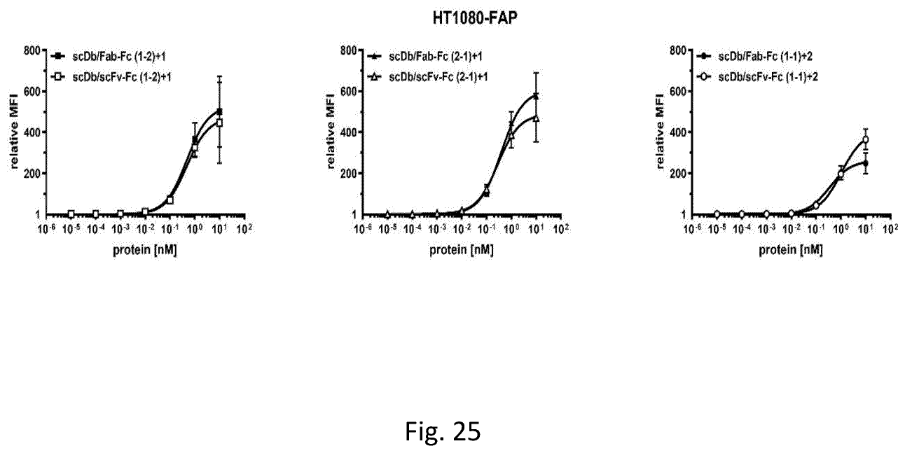

Binding of trivalent, bispecific fusion proteins to FAP-expressing (HT1080-FAP) cell lines was analyzed by flow cytometry. 2×10⁵ cells/well were incubated with a serial dilution of trivalent, bispecific antibodies for 1 h at 4° C. followed by detection using a PE-conjugated anti-human Fc antibody (Jackson ImmunoResearch Laboratories Inc). All trivalent, bispecific antibodies showed binding to FAP-expressing target cells in a concentration-dependent manner. Regarding the FAP-expressing cell line HT1080-FAP, all trivalent, bispecific antibodies bound with EC₅₀ values in the subnanomolar range, although the trivalent, bispecific antibodies in the (1-1)+2 geometry showed lower fluorescence signal intensity (FIG. 25) (Table 16).

TABLE 16
Overview of cell binding of trivalent, bispecific antibodies.

| | EC₅₀ [nM] | | | | | |
|---|---|---|---|---|---|---|
| | scDb/scFv-Fc | scDb/Fab-Fc | scDb/scFv-Fc | scDb/Fab-Fc | scDb/scFv-Fc | scDb/Fab-Fc |
| cell line | (1 − 2) + 1 | (1 − 2) + 1 | (2 − 1) + 1 | (2 − 1) + 1 | (1 − 1) + 2 | (1 − 1) + 2 |
| HT1080-FAP | 0.6 ± 0.4 | 0.5 ± 0.2 | 0.3 ± 0.1 | 0.4 ± 0.1 | 1.0 ± 0.4 | 0.4 ± 0.1 |

EC₅₀ values are shown in nM.
Mean ± SD, n = 3.

1. A trivalent binding molecule comprising:
(A) a first polypeptide comprising a single-chain dual valence antigen binding polypeptide (scDVAP), wherein the scDVAP comprises a first binding domain comprising a first variable chain (VC1) and a second variable chain (VC2), and a second binding domain comprising a third variable chain (VC3) and a fourth variable chain (VC4), wherein VC1 and VC2 together form a first antigen binding site, and VC3 and VC4 together form a second antigen binding site, wherein
(i) VC1 and VC4 are connected by a first peptide linker (L1), VC4 and VC3 are connected by a second peptide linker (L2), and VC3 and VC2 are connected by a third peptide linker (L3), or
(ii) wherein VC4 and VC1 are connected by a first peptide linker (L1), VC1 and VC2 are connected by a second peptide linker (L2), and VC2 and VC3 are connected by a third peptide linker (L3),
(B) a second polypeptide comprising a third binding domain comprising a fifth variable chain (VC5) and a sixth variable chain (VC6), wherein VC5 and VC6 together form a third antigen binding site,
wherein
(a) two of the binding sites of the trivalent binding molecule specifically bind to the same or different antigens which is not a trigger molecule on an immune effector cell,
(b) only one of the binding sites of the trivalent binding molecule is directed against a trigger molecule on an immune effector cell, and
(c) the first and second polypeptide are interconnected.

2. The trivalent binding molecule according to claim 1, wherein:
(i) the first binding site of the scDVAP and the third binding site of the second polypeptide specifically bind the same or a different antigen, and the second binding site of the scDVAP specifically binds a trigger molecule on an immune effector cell; or
(ii) the first binding site of the scDVAP and the second binding site of the scDVAP specifically bind the same or a different antigen, and the third binding site of the second binding module specifically binds a trigger molecule on an immune effector cell.

3. The trivalent binding molecule according to claim 1, wherein the variable chains (VCs) are each selected from the group consisting of a TCR α-chain variable domain, TCR β-chain variable domain, variable light (V_{L}) chain domain and variable heavy (V_{H}) chain domain.

4. The trivalent binding molecule according to claim 1, wherein the scDVAP is a single chain diabody.

5. The trivalent binding molecule according to claim 1,
wherein VC5 and VC6 are connected by a fourth peptide linker (L4), and/or
wherein the two binding sites specifically binding to antigens bind the same antigen.

6. The trivalent binding molecule according to claim 1, wherein the second polypeptide is selected from the group consisting of a single variable heavy or light chain domain, an scFv, and a Fab fragment.

7. The trivalent binding molecule according to any of claim 1, wherein the first and second polypeptides are interconnected by a fifth peptide linker (L5), a peptide bond, a disulfide bond or by one or more dimerization domains.

8. The trivalent binding molecule according to claim 7, wherein the one or more dimerization domain is selected from the group consisting of an Fc region, a heterodimerizing Fc region, CH1/CL, EHD2, MHD2, hetEHD2, the last heavy chain domain (CH3 or CH4) of IgG, IgD, IgA, IgM, or IgE and heterodimerizing derivatives thereof, and the constant C-alpha and C-beta domains of a T cell receptor (TCR), preferably wherein the one or more dimerization domain is an effector-deficient Fc region.

9. The trivalent binding molecule according to claim 8, wherein the first binding module is connected, preferably via a peptide bond or a linker (L6), to a first heterodimerizing domain, and the second binding module is connected, preferably via a peptide bond or a linker (L7), to the same or a second heterodimerizing domain, preferably wherein the heterodimerizing domains of the first and second polypeptide bind to each other through hydrophobic and/or electrostatic interactions.

10. The trivalent binding molecule according to claim 1,
wherein the immune effector cell is selected from the group consisting of T-cells, natural killer cells, natural killer T cells, macrophages, and granulocytes, and/or
wherein the trigger molecule of the immune effector cell is selected from the group consisting of CD2, CD3, CD16, CD44, CD64, CD69, CD89, Mel14, or Ly-6.2C, and/or
wherein the antigen is a tumor-associated antigen, preferably wherein the tumor-associated antigen is selected from the group consisting of EGFR, EGFRvIII, HER2, HER3, HER4, cMET, RON, FGFR2, FGFR3, IGF-1R, AXL, Tyro-3 MerTK, ALK, ROS-1, ROR-1, ROR-2, RET, MCSP, FAP, Endoglin, EpCAM, claudin-6, claudin 18.2, CD19, CD20, CD22, CD30, CD33, CD52, CD38, CD123, BCMA, CEA, PSMA, DLL3, FLT3, gpA33, SLAM-7, CCR9.

11. A nucleic acid or set of nucleic acids encoding the trivalent binding molecule according to claim 1.

12. A vector comprising the nucleic acid or set of nucleic acids of claim 11.

13. A pharmaceutical composition comprising the trivalent binding molecule according to claim 1, and a pharmaceutically acceptable carrier.

14. (canceled)

15. A method for treating cancer, a viral infection or an autoimmune disease, comprising administering to a subject in need thereof the trivalent binding molecule according to claim 1, preferably wherein the cancer is selected from the group consisting of carcinoma, sarcoma, lymphoma, leukemia, germ cell tumor and blastoma.


SEQUENCE LISTING (38 sequences)

SEQ ID NO: 1 - protein, 5 aa, Artificial Sequence
  Linker01
GGGGS

SEQ ID NO: 2 - protein, 15 aa, Artificial Sequence
  Linker02
GGGGSGGGGSGGGGS

SEQ ID NO: 3 - protein, 15 aa, Artificial Sequence
  Linker03
AAAGGSGGGGSGGGT

SEQ ID NO: 4 - protein, 4 aa, Artificial Sequence
  Linker04
  misc_feature (4)..(4): Xaa can be any naturally occurring amino acid
AAAX

SEQ ID NO: 5 - protein, 495 aa, Artificial Sequence
  Construct01
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRGGGGSGGGGSGGGGSQVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSQAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLAAAHHHHHH

SEQ ID NO: 6 - protein, 756 aa, Artificial Sequence
  Construct02
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRGGGGSGGGGSGGGGSQVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSQAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLAAAGGSGGGGSGGGTQVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSGGGGSGGGGSGGGGSQAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLGSLHHHHHH

SEQ ID NO: 7 - protein, 715 aa, Artificial Sequence
  Construct03
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRGGGGSGGGGSGGGGSQVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSQAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - protein, 715 aa, Artificial Sequence
  Construct04
QVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSQAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLGGGGSGGGGSGGGGSQVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 475 aa, Artificial Sequence
  Construct05
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSGGGGSGGGGSGGGGSQAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 454 aa, Artificial Sequence
  Construct06
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGTDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, Artificial Sequence
  Construct07
QAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 716 aa, Artificial Sequence
  Construct08
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSGGGGSQAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLGGGGSGGGGSGGGGSQVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSGGGGSQAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 474 aa, Artificial Sequence
  Construct09
DIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRGGGGSGGGGSGGGGSQVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 387 aa, Artificial Sequence
  Construct10
KDRVTITADKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 214 aa, Artificial Sequence
  Construct11
DIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 20 aa, Artificial Sequence
  Leader
METDTLLLWVLLLWVPGSTG

SEQ ID NO: 17 - protein, 16 aa, Artificial Sequence
  Hinge
DKTHTCPPCPAPPVAG

SEQ ID NO: 18 - protein, 747 aa, Artificial Sequence
  scDbhu225xhuU3-scFvhu225-His
EVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRGGGGSGGGGSGGGGSQVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSDIQLTQSPSFLSASVGDRVTITCRASQSIGTNIHWYQQKPGKAPKLLIKYASESISGVPSRFSGSGSGTEFTLTISSLQPEDFATYYCQQNNNWPTTFGAGTKLEIKRAAAGGSGGGGSGGGTEVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSGGGGSGGGGSGGGGSDIQLTQSPSFLSASVGDRVTITCRASQSIGTNIHWYQQKPGKAPKLLIKYASESISGVPSRFSGSGSGTEFTLTISSLQPEDFATYYCQQNNNWPTTFGAGTKLEIKRSLHHHHHH

SEQ ID NO: 19 - protein, 509 aa, Artificial Sequence
  scDbhu225xhuU3-His
EVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRGGGGSGGGGSGGGGSQVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSDIQLTQSPSFLSASVGDRVTITCRASQSIGTNIHWYQQKPGKAPKLLIKYASESISGVPSRFSGSGSGTEFTLTISSLQPEDFATYYCQQNNNWPTTFGAGTKLEIKRAAAGGSGGGGSGGGTGGGGSLHHHHHH

SEQ ID NO: 20 - protein, 711 aa, Artificial Sequence
  scDbCEAxhuU3-Fc(hole)delta-ab
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRGGGGSGGGGSGGGGSQVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSDIELTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 711 aa, Artificial Sequence
  scDbhuU3xCEA-Fc(hole)delta-ab
QVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSDIELTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRGGGGSGGGGSGGGGSQVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 471 aa, Artificial Sequence
  scFvCEA-Fc(knob)delta-ab
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSDIELTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 449 aa, Artificial Sequence
  VHCEA-CH1-Fc(knob)delta-ab
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, Artificial Sequence
  VLCEA-CLkappa
DIELTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 708 aa, Artificial Sequence
  scDbCEAxCEA-Fc(hole)delta-ab
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSDIELTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRGGGGSGGGGSGGGGSQVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSDIELTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 26 - protein, 711 aa, Artificial Sequence
  scDbhu225xhuU3-Fc(hole)delta-ab
EVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRGGGGSGGGGSGGGGSQVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSDIQLTQSPSFLSASVGDRVTITCRASQSIGTNIHWYQQKPGKAPKLLIKYASESISGVPSRFSGSGSGTEFTLTISSLQPEDFATYYCQQNNNWPTTFGAGTKLEIKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 711 aa, Artificial Sequence
  scDbhuU3xhu225-Fc(hole)delta-ab
QVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSDIQLTQSPSFLSASVGDRVTITCRASQSIGTNIHWYQQKPGKAPKLLIKYASESISGVPSRFSGSGSGTEFTLTISSLQPEDFATYYCQQNNNWPTTFGAGTKLEIKRGGGGSGGGGSGGGGSEVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - protein, 471 aa, Artificial Sequence
  scFvhu225-Fc(knob)delta-ab
EVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSGGGGSGGGGSGGGGSDIQLTQSPSFLSASVGDRVTITCRASQSIGTNIHWYQQKPGKAPKLLIKYASESISGVPSRFSGSGSGTEFTLTISSLQPEDFATYYCQQNNNWPTTFGAGTKLEIKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 450 aa, Artificial Sequence
  Fdhu225-Fc(knob)delta-ab
EVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGTDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 214 aa, Artificial Sequence
  VLhu225-CLkappa
DIQLTQSPSFLSASVGDRVTITCRASQSIGTNIHWYQQKPGKAPKLLIKYASESISGVPSRFSGSGSGTEFTLTISSLQPEDFATYYCQQNNNWPTTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 708 aa, Artificial Sequence
  scDbhu225xhu225-Fc(hole)delta-ab
EVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSGGGGSDIQLTQSPSFLSASVGDRVTITCRASQSIGTNIHWYQQKPGKAPKLLIKYASESISGVPSRFSGSGSGTEFTLTISSLQPEDFATYYCQQNNNWPTTFGAGTKLEIKRGGGGSGGGGSGGGGSEVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSGGGGSDIQLTQSPSFLSASVGDRVTITCRASQSIGTNIHWYQQKPGKAPKLLIKYASESISGVPSRFSGSGSGTEFTLTISSLQPEDFATYYCQQNNNWPTTFGAGTKLEIKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 32 - protein, 712 aa, Artificial Sequence
  scDbhu225x3-43-Fc(hole)delta-ab
EVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSGGGGSQAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLGGGGSGGGGSGGGGSQVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSGGGGSDIQLTQSPSFLSASVGDRVTITCRASQSIGTNIHWYQQKPGKAPKLLIKYASESISGVPSRFSGSGSGTEFTLTISSLQPEDFATYYCQQNNNWPTTFGAGTKLEIKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 716 aa, Artificial Sequence
  scDbFAPxCD3-Fc(hole)delta-ab
QVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRGGGGSGGGGSGGGGSQVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSDIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 34 - protein, 709 aa, Artificial Sequence
  scDbCD3xFAP-Fc(hole)delta-ab
QVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSGGGGSDIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRGGGGSGGGGSQVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVGGGGSDIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 476 aa, Artificial Sequence
  scFvFAP-Fc(knob)delta-ab
QVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSGGGGSGGGGSGGGGSDIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 449 aa, Artificial Sequence
  VHFAP-CH1-Fc(knob)delta-ab
QVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 219 aa, Artificial Sequence
  VLFAP-CLkappa
DIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 711 aa, Artificial Sequence
  scDbFAPxFAP-Fc(hole)delta-ab
QVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSGGGGSDIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRGGGGSGGGGSQVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVGGGGSDIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRAAADKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK